SEQUENCE LISTING (7 sequences)

SEQ ID NO: 1 - protein, 129 aa, Homo sapiens
MKTLQFFFLFCCWKAICCNSCELTNITIAIEKEECRFCISINTTWCAGYCYTRDLVYKDPARPKIQKTCTFKELVYETVRVPGCAHHADSLYTYPVATQCHCGKCDSDSTDCTVRGLGPSYCSFGEMKE

SEQ ID NO: 2 - protein, 124 aa, Homo sapiens
MPACRLGPLAAALLLSLLLFGFTLVSGTGAEKTGVCPELQADQNCTQECVSDSECADNLKCCSAGCATFCSLPNDKEGSCPQVNINFPQLGLCRDQCQVDSQCPGQMKCCRNGCGKVSCVTPNF

SEQ ID NO: 3 - protein, 22152 aa, Homo sapiens
  MOD_RES (13877)..(13878): Any amino acid
  MOD_RES (13880)..(13880): Any amino acid
  MOD_RES (13887)..(13887): Any amino acid
  MOD_RES (13890)..(13891): Any amino acid
  MOD_RES (13893)..(13893): Any amino acid
  MOD_RES (13903)..(13903): Any amino acid
  MOD_RES (13913)..(13914): Any amino acid
  MOD_RES (13916)..(13916): Any amino acid
  MOD_RES (13928)..(13929): Any amino acid
  MOD_RES (13938)..(13938): Any amino acid
  MOD_RES (13940)..(13941): Any amino acid
  MOD_RES (14569)..(14571): Any amino acid
  MOD_RES (14575)..(14575): Any amino acid
  MOD_RES (14579)..(14579): Any amino acid
  MOD_RES (14581)..(14581): Any amino acid
  MOD_RES (14587)..(14591): Any amino acid
  MOD_RES (14593)..(14594): Any amino acid
  MOD_RES (14725)..(14727): Any amino acid
  MOD_RES (14731)..(14731): Any amino acid
  MOD_RES (14735)..(14735): Any amino acid
  MOD_RES (14737)..(14737): Any amino acid
  MOD_RES (14743)..(14747): Any amino acid
  MOD_RES (14749)..(14750): Any amino acid
  MOD_RES (15661)..(15663): Any amino acid
  MOD_RES (15667)..(15667): Any amino acid
  MOD_RES (15671)..(15671): Any amino acid
  MOD_RES (15673)..(15673): Any amino acid
  MOD_RES (15679)..(15683): Any amino acid
  MOD_RES (15685)..(15686): Any amino acid
  MOD_RES (15972)..(15974): Any amino acid
  MOD_RES (15978)..(15978): Any amino acid
  MOD_RES (15982)..(15982): Any amino acid
  MOD_RES (15984)..(15984): Any amino acid
  MOD_RES (15990)..(15994): Any amino acid
  MOD_RES (15996)..(15997): Any amino acid
  MOD_RES (16008)..(16008): Any amino acid
  MOD_RES (16015)..(16015): Any amino acid
  MOD_RES (16017)..(16017): Any amino acid
  MOD_RES (16021)..(16021): Any amino acid
  MOD_RES (16025)..(16025): Any amino acid
  MOD_RES (16034)..(16034): Any amino acid
  MOD_RES (16037)..(16037): Any amino acid
  MOD_RES (16040)..(16040): Any amino acid
  MOD_RES (16046)..(16046): Any amino acid
  MOD_RES (16051)..(16051): Any amino acid
  MOD_RES (16053)..(16055): Any amino acid
  MOD_RES (16058)..(16058): Any amino acid
  MOD_RES (16062)..(16063): Any amino acid
  MOD_RES (16065)..(16065): Any amino acid
  MOD_RES (16072)..(16072): Any amino acid
  MOD_RES (16075)..(16076): Any amino acid
  MOD_RES (16078)..(16078): Any amino acid
  MOD_RES (16088)..(16088): Any amino acid
  MOD_RES (16268)..(16269): Any amino acid
  MOD_RES (16278)..(16278): Any amino acid
  MOD_RES (16280)..(16281): Any amino acid
  MOD_RES (16373)..(16374): Any amino acid
  MOD_RES (16376)..(16376): Any amino acid
  MOD_RES (16383)..(16383): Any amino acid
  MOD_RES (16386)..(16387): Any amino acid
  MOD_RES (16389)..(16389): Any amino acid
  MOD_RES (16399)..(16399): Any amino acid
  MOD_RES (16409)..(16410): Any amino acid
  MOD_RES (16412)..(16412): Any amino acid
  MOD_RES (16424)..(16425): Any amino acid
  MOD_RES (16434)..(16434): Any amino acid
  MOD_RES (16436)..(16437): Any amino acid
  MOD_RES (16439)..(16441): Any amino acid
  MOD_RES (16445)..(16445): Any amino acid
  MOD_RES (16449)..(16449): Any amino acid
  MOD_RES (16451)..(16451): Any amino acid
  MOD_RES (16457)..(16461): Any amino acid
  MOD_RES (16463)..(16464): Any amino acid
  MOD_RES (16841)..(16842): Any amino acid
  MOD_RES (16844)..(16844): Any amino acid
  MOD_RES (16851)..(16851): Any amino acid
  MOD_RES (16854)..(16855): Any amino acid
  MOD_RES (16857)..(16857): Any amino acid
  MOD_RES (16867)..(16867): Any amino acid
  MOD_RES (16877)..(16878): Any amino acid
  MOD_RES (16880)..(16880): Any amino acid
  MOD_RES (16892)..(16893): Any amino acid
  MOD_RES (16902)..(16902): Any amino acid
  MOD_RES (16904)..(16905): Any amino acid
  MOD_RES (16907)..(16909): Any amino acid
  MOD_RES (16913)..(16913): Any amino acid
  MOD_RES (16917)..(16917): Any amino acid
  MOD_RES (16919)..(16919): Any amino acid
  MOD_RES (16925)..(16929): Any amino acid
  MOD_RES (16931)..(16932): Any amino acid
  MOD_RES (17465)..(17466): Any amino acid
  MOD_RES (17468)..(17468): Any amino acid
  MOD_RES (17475)..(17475): Any amino acid
  MOD_RES (17478)..(17479): Any amino acid
  MOD_RES (17481)..(17481): Any amino acid
  MOD_RES (17491)..(17491): Any amino acid
  MOD_RES (17501)..(17502): Any amino acid
  MOD_RES (17504)..(17504): Any amino acid
  MOD_RES (17516)..(17517): Any amino acid
  MOD_RES (17526)..(17526): Any amino acid
  MOD_RES (17528)..(17529): Any amino acid
  MOD_RES (17531)..(17533): Any amino acid
  MOD_RES (17537)..(17537): Any amino acid
  MOD_RES (17541)..(17541): Any amino acid
  MOD_RES (17543)..(17543): Any amino acid
  MOD_RES (17549)..(17553): Any amino acid
  MOD_RES (17555)..(17556): Any amino acid
  MOD_RES (17567)..(17567): Any amino acid
  MOD_RES (17574)..(17574): Any amino acid
  MOD_RES (17576)..(17576): Any amino acid
  MOD_RES (17580)..(17580): Any amino acid
  MOD_RES (17584)..(17584): Any amino acid
  MOD_RES (17593)..(17593): Any amino acid
  MOD_RES (17596)..(17596): Any amino acid
  MOD_RES (17599)..(17599): Any amino acid
  MOD_RES (17605)..(17605): Any amino acid
  MOD_RES (17610)..(17610): Any amino acid
  MOD_RES (17612)..(17614): Any amino acid
  MOD_RES (17617)..(17617): Any amino acid
  MOD_RES (17621)..(17622): Any amino acid
  MOD_RES (17624)..(17624): Any amino acid
  MOD_RES (17631)..(17631): Any amino acid
  MOD_RES (17777)..(17778): Any amino acid
  MOD_RES (17780)..(17780): Any amino acid
  MOD_RES (17787)..(17787): Any amino acid
  MOD_RES (17790)..(17791): Any amino acid
  MOD_RES (17793)..(17793): Any amino acid
  MOD_RES (17803)..(17803): Any amino acid
  MOD_RES (17813)..(17814): Any amino acid
  MOD_RES (17816)..(17816): Any amino acid
  MOD_RES (17828)..(17829): Any amino acid
  MOD_RES (17838)..(17838): Any amino acid
  MOD_RES (17840)..(17841): Any amino acid
  MOD_RES (17843)..(17845): Any amino acid
  MOD_RES (17849)..(17849): Any amino acid
  MOD_RES (17853)..(17853): Any amino acid
  MOD_RES (17855)..(17855): Any amino acid
  MOD_RES (17861)..(17865): Any amino acid
  MOD_RES (17867)..(17868): Any amino acid
  MOD_RES (17879)..(17879): Any amino acid
  MOD_RES (17886)..(17886): Any amino acid
  MOD_RES (17888)..(17888): Any amino acid
  MOD_RES (17892)..(17892): Any amino acid
  MOD_RES (17896)..(17896): Any amino acid
  MOD_RES (17905)..(17905): Any amino acid
  MOD_RES (17908)..(17908): Any amino acid
  MOD_RES (17911)..(17911): Any amino acid
  MOD_RES (17917)..(17917): Any amino acid
  MOD_RES (17922)..(17922): Any amino acid
  MOD_RES (17924)..(17926): Any amino acid
  MOD_RES (17929)..(17929): Any amino acid
  MOD_RES (17933)..(17934): Any amino acid
  MOD_RES (17936)..(17936): Any amino acid
  MOD_RES (17943)..(17943): Any amino acid
  MOD_RES (17946)..(17947): Any amino acid
  MOD_RES (17949)..(17949): Any amino acid
  MOD_RES (17959)..(17959): Any amino acid
  MOD_RES (18089)..(18090): Any amino acid
  MOD_RES (18092)..(18092): Any amino acid
  MOD_RES (18099)..(18099): Any amino acid
  MOD_RES (18102)..(18103): Any amino acid
  MOD_RES (18105)..(18105): Any amino acid
  MOD_RES (18115)..(18115): Any amino acid
  MOD_RES (18125)..(18126): Any amino acid
  MOD_RES (18128)..(18128): Any amino acid
  MOD_RES (18140)..(18141): Any amino acid
  MOD_RES (18150)..(18150): Any amino acid
  MOD_RES (18152)..(18153): Any amino acid
  MOD_RES (18155)..(18157): Any amino acid
  MOD_RES (18161)..(18161): Any amino acid
  MOD_RES (18165)..(18165): Any amino acid
  MOD_RES (18167)..(18167): Any amino acid
  MOD_RES (18173)..(18177): Any amino acid
  MOD_RES (18179)..(18180): Any amino acid
  MOD_RES (18191)..(18191): Any amino acid
  MOD_RES (18198)..(18198): Any amino acid
  MOD_RES (18200)..(18200): Any amino acid
  MOD_RES (18204)..(18204): Any amino acid
  MOD_RES (18208)..(18208): Any amino acid
  MOD_RES (18217)..(18217): Any amino acid
  MOD_RES (18220)..(18220): Any amino acid
  MOD_RES (18223)..(18223): Any amino acid
  MOD_RES (18229)..(18229): Any amino acid
  MOD_RES (18234)..(18234): Any amino acid
  MOD_RES (18236)..(18238): Any amino acid
  MOD_RES (18241)..(18241): Any amino acid
  MOD_RES (18245)..(18246): Any amino acid
  MOD_RES (18248)..(18248): Any amino acid
  MOD_RES (18255)..(18255): Any amino acid
  MOD_RES (18258)..(18259): Any amino acid
  MOD_RES (18261)..(18261): Any amino acid
  MOD_RES (18271)..(18271): Any amino acid
  MOD_RES (18401)..(18402): Any amino acid
  MOD_RES (18404)..(18404): Any amino acid
  MOD_RES (18411)..(18411): Any amino acid
  MOD_RES (18414)..(18415): Any amino acid
  MOD_RES (18417)..(18417): Any amino acid
  MOD_RES (18427)..(18427): Any amino acid
  MOD_RES (18437)..(18438): Any amino acid
  MOD_RES (18440)..(18440): Any amino acid
  MOD_RES (18452)..(18453): Any amino acid
  MOD_RES (18462)..(18462): Any amino acid
  MOD_RES (18464)..(18465): Any amino acid
  MOD_RES (18467)..(18469): Any amino acid
  MOD_RES (18473)..(18473): Any amino acid
  MOD_RES (18477)..(18477): Any amino acid
  MOD_RES (18479)..(18479): Any amino acid
  MOD_RES (18485)..(18489): Any amino acid
  MOD_RES (18491)..(18492): Any amino acid
  MOD_RES (18503)..(18503): Any amino acid
  MOD_RES (18510)..(18510): Any amino acid
  MOD_RES (18512)..(18512): Any amino acid
  MOD_RES (18516)..(18516): Any amino acid
  MOD_RES (18520)..(18520): Any amino acid
  MOD_RES (18529)..(18529): Any amino acid
  MOD_RES (18532)..(18532): Any amino acid
  MOD_RES (18535)..(18535): Any amino acid
  MOD_RES (18541)..(18541): Any amino acid
  MOD_RES (18546)..(18546): Any amino acid
  MOD_RES (18548)..(18550): Any amino acid
  MOD_RES (18553)..(18553): Any amino acid
  MOD_RES (18557)..(18558): Any amino acid
  MOD_RES (18560)..(18560): Any amino acid
  MOD_RES (18567)..(18567): Any amino acid
  MOD_RES (18570)..(18571): Any amino acid
  MOD_RES (18573)..(18573): Any amino acid
  MOD_RES (18583)..(18583): Any amino acid
  MOD_RES (18713)..(18714): Any amino acid
  MOD_RES (18716)..(18716): Any amino acid
  MOD_RES (18723)..(18723): Any amino acid
  MOD_RES (18726)..(18727): Any amino acid
  MOD_RES (18729)..(18729): Any amino acid
  MOD_RES (18739)..(18739): Any amino acid
  MOD_RES (18749)..(18750): Any amino acid
  MOD_RES (18752)..(18752): Any amino acid
  MOD_RES (18764)..(18765): Any amino acid
  MOD_RES (18774)..(18774): Any amino acid
  MOD_RES (18776)..(18777): Any amino acid
  MOD_RES (18779)..(18781): Any amino acid
  MOD_RES (18785)..(18785): Any amino acid
  MOD_RES (18789)..(18789): Any amino acid
  MOD_RES (18791)..(18791): Any amino acid
  MOD_RES (18797)..(18801): Any amino acid
  MOD_RES (18803)..(18804): Any amino acid
  MOD_RES (18815)..(18815): Any amino acid
  MOD_RES (18822)..(18822): Any amino acid
  MOD_RES (18824)..(18824): Any amino acid
  MOD_RES (18828)..(18828): Any amino acid
  MOD_RES (18832)..(18832): Any amino acid
  MOD_RES (18841)..(18841): Any amino acid
  MOD_RES (18844)..(18844): Any amino acid
  MOD_RES (18847)..(18847): Any amino acid
  MOD_RES (18853)..(18853): Any amino acid
  MOD_RES (18858)..(18858): Any amino acid
  MOD_RES (18860)..(18862): Any amino acid
  MOD_RES (18865)..(18865): Any amino acid
  MOD_RES (18869)..(18870): Any amino acid
  MOD_RES (18872)..(18872): Any amino acid
  MOD_RES (18879)..(18879): Any amino acid
  MOD_RES (18882)..(18883): Any amino acid
  MOD_RES (18885)..(18885): Any amino acid
  MOD_RES (18895)..(18895): Any amino acid
  MOD_RES (19091)..(19093): Any amino acid
  MOD_RES (19097)..(19097): Any amino acid
  MOD_RES (19101)..(19101): Any amino acid
  MOD_RES (19103)..(19103): Any amino acid
  MOD_RES (19109)..(19113): Any amino acid
  MOD_RES (19115)..(19116): Any amino acid
  MOD_RES (19127)..(19127): Any amino acid
  MOD_RES (19134)..(19134): Any amino acid
  MOD_RES (19136)..(19136): Any amino acid
  MOD_RES (19140)..(19140): Any amino acid
  MOD_RES (19144)..(19144): Any amino acid
  MOD_RES (19153)..(19153): Any amino acid
  MOD_RES (19156)..(19156): Any amino acid
  MOD_RES (19159)..(19159): Any amino acid
  MOD_RES (19165)..(19165): Any amino acid
  MOD_RES (19170)..(19170): Any amino acid
  MOD_RES (19172)..(19174): Any amino acid
  MOD_RES (19177)..(19177): Any amino acid
  MOD_RES (19181)..(19182): Any amino acid
  MOD_RES (19184)..(19184): Any amino acid
  MOD_RES (19191)..(19191): Any amino acid
  MOD_RES (19194)..(19195): Any amino acid
  MOD_RES (19197)..(19197): Any amino acid
  MOD_RES (19207)..(19207): Any amino acid
  MOD_RES (19337)..(19338): Any amino acid
  MOD_RES (19340)..(19340): Any amino acid
  MOD_RES (19347)..(19347): Any amino acid
  MOD_RES (19350)..(19351): Any amino acid
  MOD_RES (19353)..(19353): Any amino acid
  MOD_RES (19363)..(19363): Any amino acid
  MOD_RES (19373)..(19374): Any amino acid
  MOD_RES (19376)..(19376): Any amino acid
  MOD_RES (19388)..(19389): Any amino acid
  MOD_RES (19398)..(19398): Any amino acid
  MOD_RES (19400)..(19401): Any amino acid
  MOD_RES (19403)..(19405): Any amino acid
  MOD_RES (19409)..(19409): Any amino acid
  MOD_RES (19413)..(19413): Any amino acid
  MOD_RES (19415)..(19415): Any amino acid
  MOD_RES (19421)..(19425): Any amino acid
  MOD_RES (19427)..(19428): Any amino acid
  MOD_RES (19439)..(19439): Any amino acid
  MOD_RES (19446)..(19446): Any amino acid
  MOD_RES (19448)..(19448): Any amino acid
  MOD_RES (19452)..(19452): Any amino acid
  MOD_RES (19456)..(19456): Any amino acid
  MOD_RES (19465)..(19465): Any amino acid
  MOD_RES (19468)..(19468): Any amino acid
  MOD_RES (19471)..(19471): Any amino acid
  MOD_RES (19477)..(19477): Any amino acid
  MOD_RES (19482)..(19482): Any amino acid
  MOD_RES (19484)..(19486): Any amino acid
  MOD_RES (19489)..(19489): Any amino acid
  MOD_RES (19493)..(19494): Any amino acid
  MOD_RES (19496)..(19496): Any amino acid
  MOD_RES (19503)..(19503): Any amino acid
  MOD_RES (19506)..(19507): Any amino acid
  MOD_RES (19509)..(19509): Any amino acid
  MOD_RES (19519)..(19519): Any amino acid
  MOD_RES (19649)..(19650): Any amino acid
  MOD_RES (19652)..(19652): Any amino acid
  MOD_RES (19659)..(19659): Any amino acid
  MOD_RES (19662)..(19663): Any amino acid
  MOD_RES (19665)..(19665): Any amino acid
  MOD_RES (19675)..(19675): Any amino acid
  MOD_RES (19685)..(19686): Any amino acid
  MOD_RES (19688)..(19688): Any amino acid
  MOD_RES (19700)..(19701): Any amino acid
  MOD_RES (19710)..(19710): Any amino acid
  MOD_RES (19712)..(19713): Any amino acid
  MOD_RES (19715)..(19717): Any amino acid
  MOD_RES (19721)..(19721): Any amino acid
  MOD_RES (19725)..(19725): Any amino acid
  MOD_RES (19727)..(19727): Any amino acid
  MOD_RES (19733)..(19737): Any amino acid
  MOD_RES (19739)..(19740): Any amino acid
  MOD_RES (19751)..(19751): Any amino acid
  MOD_RES (19758)..(19758): Any amino acid
  MOD_RES (19760)..(19760): Any amino acid
  MOD_RES (19764)..(19764): Any amino acid
  MOD_RES (19768)..(19768): Any amino acid
  MOD_RES (19777)..(19777): Any amino acid
  MOD_RES (19780)..(19780): Any amino acid
  MOD_RES (19783)..(19783): Any amino acid
  MOD_RES (19789)..(19789): Any amino acid
  MOD_RES (19794)..(19794): Any amino acid
  MOD_RES (19796)..(19798): Any amino acid
  MOD_RES (19801)..(19801): Any amino acid
  MOD_RES (19805)..(19806): Any amino acid
  MOD_RES (19808)..(19808): Any amino acid
  MOD_RES (19815)..(19815): Any amino acid
  MOD_RES (19818)..(19819): Any amino acid
  MOD_RES (19821)..(19821): Any amino acid
  MOD_RES (19831)..(19831): Any amino acid
  MOD_RES (19960)..(19961): Any amino acid
  MOD_RES (19963)..(19963): Any amino acid
  MOD_RES (19970)..(19970): Any amino acid
  MOD_RES (19973)..(19974): Any amino acid
  MOD_RES (19976)..(19976): Any amino acid
  MOD_RES (19986)..(19986): Any amino acid
  MOD_RES (19996)..(19997): Any amino acid
  MOD_RES (19999)..(19999): Any amino acid
  MOD_RES (20011)..(20012): Any amino acid
  MOD_RES (20021)..(20021): Any amino acid
  MOD_RES (20023)..(20024): Any amino acid
  MOD_RES (20026)..(20028): Any amino acid
  MOD_RES (20032)..(20032): Any amino acid
  MOD_RES (20036)..(20036): Any amino acid
  MOD_RES (20038)..(20038): Any amino acid
  MOD_RES (20044)..(20048): Any amino acid
  MOD_RES (20050)..(20051): Any amino acid
  MOD_RES (20062)..(20062): Any amino acid
  MOD_RES (20069)..(20069): Any amino acid
  MOD_RES (20071)..(20071): Any amino acid
  MOD_RES (20075)..(20075): Any amino acid
  MOD_RES (20079)..(20079): Any amino acid
  MOD_RES (20088)..(20088): Any amino acid
  MOD_RES (20091)..(20091): Any amino acid
  MOD_RES (20094)..(20094): Any amino acid
  MOD_RES (20100)..(20100): Any amino acid
  MOD_RES (20105)..(20105): Any amino acid
  MOD_RES (20107)..(20109): Any amino acid
  MOD_RES (20112)..(20112): Any amino acid
  MOD_RES (20116)..(20117): Any amino acid
  MOD_RES (20119)..(20119): Any amino acid
  MOD_RES (20126)..(20126): Any amino acid
  MOD_RES (20129)..(20130): Any amino acid
  MOD_RES (20132)..(20132): Any amino acid
  MOD_RES (20142)..(20142): Any amino acid
  MOD_RES (20272)..(20273): Any amino acid
  MOD_RES (20275)..(20275): Any amino acid
  MOD_RES (20282)..(20282): Any amino acid
  MOD_RES (20285)..(20286): Any amino acid
  MOD_RES (20288)..(20288): Any amino acid
  MOD_RES (20298)..(20298): Any amino acid
  MOD_RES (20308)..(20309): Any amino acid
  MOD_RES (20311)..(20311): Any amino acid
  MOD_RES (20323)..(20324): Any amino acid
  MOD_RES (20333)..(20333): Any amino acid
  MOD_RES (20335)..(20336): Any amino acid
  MOD_RES (20806)..(20808): Any amino acid
  MOD_RES (20812)..(20812): Any amino acid
  MOD_RES (20816)..(20816): Any amino acid
  MOD_RES (20818)..(20818): Any amino acid
  MOD_RES (20824)..(20828): Any amino acid
  MOD_RES (20830)..(20831): Any amino acid
MLKPSGLPGSSSPTRSLMTGSRSTKATPEMDSGLTGATLSPKTSTGAIVVTEHTLPFTSPDKTLASPTSSVVGRTTQSLGVMSSALPESTSRGMTHSEQRTSPSLSPQVNGTPSRNYPATSMVSGLSSPRTRTSSTEGNFTKEASTYTLTVETTSGPVTEKYTVPTETSTTEGDSTETPWDTRYIPVKITSPMKTFADSTASKENAPVSMTPAETTVTDSHTPGRTNPSFGTLYSSFLDLSPKGTPNSRGETSLELILSTTGYPFSSPEPGSAGHSRISTSAPLSSSASVLDNKISETSIFSGQSLTSPLSPGVPEARASTMPNSAIPFSMTLSNAETSAERVRSTISSLGTPSISTKQTAETILTFHAFAETMDIPSTHIAKTLASEWLGSPGTLGGTSTSALTTTSPSTTLVSEETNTHHSTSGKETEGTLNTSMTPLETSAPGEESEMTATLVPTLGFTTLDSKIRSPSQVSSSHPTRELRTTGSTSGRQSSSTAAHGSSDILRATTSSTSKASSWTSESTAQQFSEPQHTQWVETSPSMKTERPPASTSVAAPITTSVPSVVSGFTTLKTSSTKGIWLEETSADTLIGESTAGPTTHQFAVPTGISMTGGSSTRGSQGTTHLLTRATASSETSADLTLATNGVPVSVSPAVSKTAAGSSPPGGTKPSYTMVSSVIPETSSLQSSAFREGTSLGLTPLNTRHPFSSPEPDSAGHTKISTSIPLLSSASVLEDKVSATSTFSHHKATSSITTGTPEISTKTKPSSAVLSSMTLSNAATSPERVRNATSPLTHPSPSGEETAGSVLTLSTSAETTDSPNIHPTGTLTSESSESPSTLSLPSVSGVKTTFSSSTPSTHLFTSGEETEETSNPSVSQPETSVSRVRTTLASTSVPTPVFPTMDTWPTRSAQFSSSHLVSELRATSSTSVTNSTGSALPKISHLTGTATMSQTNRDTFNDSAAPQSTTWPETSPRFKTGLPSATTTVSTSATSLSATVMVSKFTSPATSSMEATSIREPSTTILTTETTNGPGSMAVASTNIPIGKGYITEGRLDTSHLPIGTTASSETSMDFTMAKESVSMSVSPSQSMDAAGSSTPGRTSQFVDTFSDDVYHLTSREITIPRDGTSSALTPQMTATHPPSPDPGSARSTWLGILSSSPSSPTPKVTMSSTFSTQRVTTSMIMDTVETSRWNMPNLPSTTSLTPSNIPTSGAIGKSTLVPLDTPSPATSLEASEGGLPTLSTYPESTNTPSIHLGAHASSESPSTIKLTMASVVKPGSYTPLTFPSIETHIHVSTARMAYSSGSSPEMTAPGETNTGSTWDPTTYITTTDPKDTSSAQVSTPHSVRTLRTTENHPKTESATPAAYSGSPKISSSPNLTSPATKAWTITDTTEHSTQLHYTKLAEKSSGFETQSAPGPVSVVIPTSPTIGSSTLELTSDVPGEPLVLAPSEQTTITLPMATWLSTSLTEEMASTDLDISSPSSPMSTFAIFPPMSTPSHELSKSEADTSAIRNTDSTTLDQHLGIRSLGRTGDLTTVPITPLTTTWTSVIEHSTQAQDTLSATMSPTHVTQSLKDQTSIPASASPSHLTEVYPELGTQGRSSSEATTFWKPSTDTLSREIETGPTNIQSTPPMDNTTTGSSSSGVTLGIAHLPIGTSSPAETSTNMALERRSSTATVSMAGTMGLLVTSAPGRSISQSLGRVSSVLSESTTEGVTDSSKGSSPRLNTQGNTALSSSLEPSYAEGSQMSTSIPLTSSPTTPDVEFIGGSTFWTKEVTTVMTSDISKSSARTESSSATLMSTALGSTENTGKEKLRTASMDLPSPTPSMEVTPWISLTLSNAPNTTDSLDLSHGVHTSSAGTLATDRSLNTGVTRASRLENGSDTSSKSLSMGNSTHTSMTDTEKSEVSSSIHPRPETSAPGAETTLTSTPGNRAISLTLPFSSIPVEEVISTGITSGPDINSAPMTHSPITPPTIVWTSTGTIEQSTQPLHAVSSEKVSVQTQSTPYVNSVAVSASPTHENSVSSGSSTSSPYSSASLESLDSTISRRNAITSWLWDLTTSLPTTTWPSTSLSEALSSGHSGVSNPSSTTTEFPLFSAASTSAAKQRNPETETHGPQNTAASTLNTDASSVTGLSETPVGASISSEVPLPMAITSRSDVSGLTSESTANPSLGTASSAGTKLTRTISLPTSESLVSFRMNKDPWTVSIPLGSHPTTNTETSIPVNSAGPPGLSTVASDVIDTPSDGAESIPTVSFSPSPDTEVTTISHFPEKTTHSFRTISSLTHELTSRVTPIPGDWMSSAMSTKPTGASPSITLGERRTITSAAPTTSPIVLTASFTETSTVSLDNETTVKTSDILDARKTNELPSDSSSSSDLINTSIASSTMDVTKTASISPTSISGMTASSSPSLFSSDRPQVPTSTTETNTATSPSVSSNTYSLDGGSNVGGTPSTLPPFTITHPVETSSALLAWSRPVRTFSTMVSTDTASGENPTSSNSVVTSVPAPGTWASVGSTTDLPAMGFLKTSPAGEAHSLLASTIEPATAFTPHLSAAVVTGSSATSEASLLTTSESKAIHSSPQTPTTPTSGANWETSATPESLLVVTETSDTTLTSKILVTDTILFSTVSTPPSKFPSTGTLSGASFPTLLPDTPAIPLTATEPTSSLATSFDSTPLVTIASDSLGTVPETTLTMSETSNGDALVLKTVSNPDRSIPGITIQGVTESPLHPSSTSPSKIVAPRNTTYEGSITVALSTLPAGTTGSLVFSQSSENSETTALVDSSAGLERASVMPLTTGSQGMASSGGIRSGSTHSTGTKTFSSLPLTMNPGEVTAMSEITTNRLTATQSTAPKGIPVKPTSAESGLLTPVSASSSPSKAFASLTTAPPSTWGIPQSTLTFEFSEVPSLDTKSASLPTPGQSLNTIPDSDASTASSSLSKSPEKNPRARMMTSTKAISASSFQSTGFTETPEGSASPSMAGHEPRVPTSGTGDPRYASESMSYPDPSKASSAMTSTSLASKLTTLFSTGQAARSGSSSSPISLSTEKETSFLSPTASTSRKTSLFLGPSMARQPNILVHLQTSALTLSPTSTLNMSQEEPPELTSSQTIAEEEGTTAETQTLTFTPSETPTSLLPVSSPTEPTARRKSSPETWASSISVPAKTSLVETTDGTLVTTIKMSSQAAQGNSTWPAPAEETGTSPAGTSPGSPEVSTTLKIMSSKEPSISPEIRSTVRNSPWKTPETTVPMETTVEPVTLQSTALGSGSTSISHLPTGTTSPTKSPTENMLATERVSLSPSPPEAWTNLYSGTPGGTRQSLATMSSVSLESPTARSITGTGQQSSPELVSKTTGMEFSMWHGSTGGTTGDTHVSLSTSSNILEDPVTSPNSVSSLTDKSKHKTETWVSTTAIPSTVLNNKIMAAEQQTSRSVDEAYSSTSSWSDQTSGSDITLGASPDVTNTLYITSTAQTTSLVSLPSGDQGITSLTNPSGGKTSSASSVTSPSIGLETLRANVSAVKSDIAPTAGHLSQTSSPAEVSILDVTTAPTPGISTTITTMGTNSISTTTPNPEVGMSTMDSTPATERRTTSTEHPSTWSSTAASDSWTVTDMTSNLKVARSPGTISTMHTTSFLASSTELDSMSTPHGRITVIGTSLVTPSSDASAVKTETSTSERTLSPSDTTASTPISTFSRVQRMSISVPDILSTSWTPSSTEAEDVPVSMVSTDHASTKTDPNTPLSTFLFDSLSTLDWDTGRSLSSATATTSAPQGATTPQELTLETMISPATSQLPFSIGHITSAVTPAAMARSSGVTFSRPDPTSKKAEQTSTQLPTTTSAHPGQVPRSAATTLDVIPHTAKTPDATFQRQGQTALTTEARATSDSWNEKEKSTPSAPWITEMMNSVSEDTIKEVTSSSSVLKDPEYAGHKLGIWDDFIPKFGKAAHMRELPLLSPPQDKEAIHPSTNTVETTGWVTSSEHASHSTIPAHSASSKLTSPVVTTSTREQAIVSMSTTTWPESTRARTEPNSFLTIELRDVSPYMDTSSTTQTSIISSPGSTAITKGPRTEITSSKRISSSFLAQSMRSSDSPSEAITRLSNFPAMTESGGMILAMQTSPPGATSLSAPTLDTSATASWTGTPLATTQRFTYSEKTTLFSKGPEDTSQPSPPSVEETSSSSSLVPIHATTSPSNILLTSQGHSPSSTPPVTSVFLSETSGLGKTTDMSRISLEPGTSLPPNLSSTAGEALSTYEASRDTKAIHHSADTAVTNMEATSSEYSPIPGHTKPSKATSPLVTSHIMGDITSSTSVFGSSETTEIETVSSVNQGLQERSTSQVASSATETSTVITHVSSGDATTHVTKTQATFSSGTSISSPHQFITSTNTFTDVSTNPSTSLIMTESSGVTITTQTGPTGAATQGPYLLDTSTMPYLTETPLAVTPDFMQSEKTTLISKGPKDVTWTSPPSVAETSYPSSLTPFLVTTIPPATSTLQGQHTSSPVSATSVLTSGLVKTTDMLNTSMEPVTNSPQNLNNPSNEILATLAATTDIETIHPSINKAVTNMGTASSAHVLHSTLPVSSEPSTATSPMVPASSMGDALASISIPGSETTDIEGEPTSSLTAGRKENSTLQEMNSTTESNIILSNVSVGAITEATKMEVPSFDATFIPTPAQSTKFPDIFSVASSRLSNSPPMTISTHMTTTQTGSSGATSKIPLALDTSTLETSAGTPSVVTEGFAHSKITTAMNNDVKDVSQTNPPFQDEASSPSSQAPVLVTTLPSSVAFTPQWHSTSSPVSMSSVLTSSLVKTAGKVDTSLETVTSSPQSMSNTLDDISVTSAATTDIETTHPSINTVVTNVGTTGSAFESHSTVSAYPEPSKVTSPNVTTSTMEDTTISRSIPKSSKTTRTETETTSSLTPKLRETSISQEITSSTETSTVPYKELTGATTEVSRTDVTSSSSTSFPGPDQSTVSLDISTETNTRLSTSPIMTESAEITITTQTGPHGATSQDTFTMDPSNTTPQAGIHSAMTHGFSQLDVTTLMSRIPQDVSWTSPPSVDKTSSPSSFLSSPAMTTPSLISSTLPEDKLSSPMTSLLTSGLVKITDILRTRLEPVTSSLPNFSSTSDKILATSKDSKDTKEIFPSINTEETNVKANNSGHESHSPALADSETPKATTQMVITTTVGDPAPSTSMPVHGSSETTNIKREPTYFLTPRLRETSTSQESSFPTDTSFLLSKVPTGTITEVSSTGVNSSSKISTPDHDKSTVPPDTFTGEIPRVFTSSIKTKSAEMTITTQASPPESASHSTLPLDTSTTLSQGGTHSTVTQGFPYSEVTTLMGMGPGNVSWMTTPPVEETSSVSSLMSSPAMTSPSPVSSTSPQSIPSSPLPVTALPTSVLVTTTDVLGTTSPESVTSSPPNLSSITHERPATYKDTAHTEAAMHHSTNTAVTNVGTSGSGHKSQSSVLADSETSKATPLMSTTSTLGDTSVSTSTPNISQTNQIQTEPTASLSPRLRESSTSEKTSSTTETNTAFSYVPTGAITQASRTEISSSRTSISDLDRPTIAPDISTGMITRLFTSPIMTKSAEMTVTTQTTTPGATSQGILPWDTSTTLFQGGTHSTVSQGFPHSEITTLRSRTPGDVSWMTTPPVEETSSGFSLMSPSMTSPSPVSSTSPESIPSSPLPVTALLTSVLVTTTNVLGTTSPETVTSSPPNLSSPTQERLTTYKDTAHTEAMHASMHTNTAVANVGTSISGHESQSSVPADSHTSKATSPMGITFAMGDTSVSTSTPAFFETRIQTESTSSLIPGLRDTRTSEEINTVTETSTVLSEVPTTTTTEVSRTEVITSSRTTISGPDHSKMSPYISTETITRLSTFPFVTGSTEMAITNQTGPIGTISQATLTLDTSSTASWEGTHSPVTQRFPHSEETTTMSRSTKGVSWQSPPSVEETSSPSSPVPLPAITSHSSLYSAVSGSSPTSALPVTSLLTSGRRKTIDMLDTHSELVTSSLPSASSFSGEILTSEASTNTETIHFSENTAETNMGTTNSMHKLHSSVSIHSQPSGHTPPKVTGSMMEDAIVSTSTPGSPETKNVDRDSTSPLTPELKEDSTALVMNSTTESNTVFSSVSLDAATEVSRAEVTYYDPTFMPASAQSTKSPDISPEASSSHSNSPPLTISTHKTIATQTGPSGVTSLGQLTLDTSTIATSAGTPSARTQDFVDSETTSVMNNDLNDVLKTSPFSAEEANSLSSQAPLLVTTSPSPVTSTLQEHSTSSLVSVTSVPTPTLAKITDMDTNLEPVTRSPQNLRNTLATSEATTDTHTMHPSINTAMANVGTTSSPNEFYFTVSPDSDPYKATSAVVITSTSGDSIVSTSMPRSSAMKKIESETTFSLIFRLRETSTSQKIGSSSDTSTVFDKAFTAATTEVSRTELTSSSRTSIQGTEKPTMSPDTSTRSVTMLSTFAGLTKSEERTIATQTGPHRATSQGTLTWDTSITTSQAGTHSAMTHGFSQLDLSTLTSRVPEYISGTSPPSVEKTSSSSSLLSLPAITSPSPVPTTLPESRPSSPVHLTSLPTSGLVKTTDMLASVASLPPNLGSTSHKIPTTSEDIKDTEKMYPSTNIAVTNVGTTTSEKESYSSVPAYSEPPKVTSPMVTSFNIRDTIVSTSMPGSSEITRIEMESTFSVAHGLKGTSTSQDPIVSTEKSAVLHKLTTGATETSRTEVASSRRTSIPGPDHSTESPDISTEVIPSLPISLGITESSNMTIITRTGPPLGSTSQGTFTLDTPTTSSRAGTHSMATQEFPHSEMTTVMNKDPEILSWTIPPSIEKTSFSSSLMPSPAMTSPPVSSTLPKTIHTTPSPMTSLLTPSLVMTTDTLGTSPEPTTSSPPNLSSTSHVILTTDEDTTAIEAMHPSTSTAATNVETTCSGHGSQSSVLTDSEKTKATAPMDTTSTMGHTTVSTSMSVSSETTKIKRESTYSLTPGLRETSISQNASFSTDTSIVLSEVPTGTTAEVSRTEVTSSGRTSIPGPSQSTVLPEISTRTMTRLFASPTMTESAEMTIPTQTGPSGSTSQDTLTLDTSTTKSQAKTHSTLTQRFPHSEMTTLMSRGPGDMSWQSSPSLENPSSLPSLLSLPATTSPPPISSTLPVTISSSPLPVTSLLTSSPVTTTDMLHTSPELVTSSPPKLSHTSDERLTTGKDTTNTEAVHPSTNTAASNVEIPSFGHESPSSALADSETSKATSPMFITSTQEDTTVAISTPHFLETSRIQKESISSLSPKLRETGSSVETSSAIETSAVLSEVSIGATTEISRTEVTSSSRTSISGSAESTMLPEISTTRKIIKFPTSPILAESSEMTIKTQTSPPGSTSESTFTLDTSTTPSLVITHSTMTQRLPHSEITTLVSRGAGDVPRPSSLPVEETSPPSSQLSLSAMISPSPVSSTLPASSHSSSASVTSPLTPGQVKTTEVLDASAEPETSSPPSLSSTSVEILATSEVTTDTEKIHPFPNTAVTKVGTSSSGHESPSSVLPDSETTKATSAMGTISIMGDTSVSTLTPALSNTRKIQSEPASSLTTRLRETSTSEETSLATEANTVLSKVSTGATTEVSRTEAISFSRTSMSGPEQSTMSQDISIGTIPRISASSVLTESAKMTITTQTGPSESTLESTLNLNTATTPSWVETHSIVIQGFPHPEMTTSMGRGPGGVSWPSPPFVKETSPPSSPLSLPAVTSPHPVSTTFLAHIPPSPLPVTSLLTSGPATTTDILGTSTEPGTSSSSSLSTTSHERLTTYKDTAHTEAVHPSTNTGGTNVATTSSGYKSQSSVLADSSPMCTTSTMGDTSVLTSTPAFLETRRIQTELASSLTPGLRESSGSEGTSSGTKMSTVLSKVPTGATTEISKEDVTSIPGPAQSTISPDISTRTVSWFSTSPVMTESAEITMNTHTSPLGATTQGTSTLATSSTTSLTMTHSTISQGFSHSQMSTLMRRGPEDVSWMSPPLLEKTRPSFSLMSSPATTSPSPVSSTLPESISSSPLPVTSLLTSGLAKTTDMLHKSSEPVTNSPANLSSTSVEILATSEVTTDTEKTHPSSNRTVTDVGTSSSGHESTSFVLADSQTSKVTSPMVITSTMEDTSVSTSTPGFFETSRIQTEPTSSLTLGLRKTSSSEGTSLATEMSTVLSGVPTGATAEVSRTEVTSSSRTSISGFAQLTVSPETSTETITRLPTSSIMTESAEMMIKTQTDPPGSTPESTHTVDISTTPNWVETHSTVTQRFSHSEMTTLVSRSPGDMLWPSQSSVEETSSASSLLSLPATTSPSPVSSTLVEDFPSASLPVTSLLTPGLVITTDRMGISREPGTSSTSNLSSTSHERLTTLEDTVDTEDMQPSTHTAVTNVRTSISGHESQSSVLSDSETPKATSPMGTTYTMGETSVSISTSDFFETSRIQIEPTSSLTSGLRETSSSERISSATEGSTVLSEVPSGATTEVSRTEVISSRGTSMSGPDQFTISPDISTEAITRLSTSPIMTESAESAITIETGSPGATSEGTLTLDTSTTTFWSGTHSTASPGFSHSEMTTLMSRTPGDVPWPSLPSVEEASSVSSSLSSPAMTSTSFFSALPESISSSPHPVTALLTLGPVKTTDMLRTSSEPETSSPPNLSSTSAEILATSEVTKDREKIHPSSNTPVVNVGTVIYKHLSPSSVLADLVTTKPTSPMATTSTLGNTSVSTSTPAFPETMMTQPTSSLTSGLREISTSQETSSATERSASLSGMPTGATTKVSRTEALSLGRTSTPGPAQSTISPEISTETITRISTPLTTTGSAEMTITPKTGHSGASSQGTFTLDTSSRASWPGTHSAATHRSPHSGMTTPMSRGPEDVSWPSRPSVEKTSPPSSLVSLSAVTSPSPLYSTPSESSHSSPLRVTSLFTPVMMKTTDMLDTSLEPVTTSPPSMNITSDESLATSKATMETEAIQLSENTAVTQMGTISARQEFYSSYPGLPEPSKVTSPVVTSSTIKDIVSTTIPASSEITRIEMESTSTLTPTPRETSTSQEIHSATKPSTVPYKALTSATIEDSMTQVMSSSRGPSPDQSTMSQDISSEVITRLSTSPIKAESTEMTITTQTGSPGATSRGTLTLDTSTTFMSGTHSTASQGFSHSQMTALMSRTPGDVPWLSHPSVEEASSASFSLSSPVMTSSSPVSSTLPDSIHSSSLPVTSLLTSGLVKTTELLGTSSEPETSSPPNLSSTSAEILATTEVTTDTEKLEMTNVVTSGYTHESPSSVLADSVTTKATSSMGITYPTGDTNVLTSTPAFSDTSRIQTKSKLSLTPGLMETSISEETSSATEKSTVLSSVPTGATTEVSRTEAISSSRTSIPGPAQSTMSSDTSMETITRISTPLTRKESTDMAITPKTGPSGATSQGTFTLDSSSTASWPGTHSATTQRFPQSVVTTPMSRGPEDVSWPSPLSVEKNSPPSSLVSSSSVTSPSPLYSTPSGSSHSSPVPVTSLFTSIMMKATDMLDASLEPETTSAPNMNITSDESLATSKATTETEAIHVFENTAASHVETTSATEELYSSSPGFSEPTKVISPVVTSSSIRDNMVSTTMPGSSGITRIEIESMSSLTPGLRETRTSQDITSSTETSTVLYKMSSGATPEVSRTEVMPSSRTSIPGPAQSTMSLDISDEVVTRLSTSPIMTESAEITITTQTGYSLATSQVTLPLGTSMTFLSGTHSTMSQGLSHSEMTNLMSRGPESLSWTSPRFVETTRSSSSLTSLPLTTSLSPVSSTLLDSSPSSPLPVTSLILPGLVKTTEVLDTSSEPKTSSSPNLSSTSVEIPATSEIMTDTEKIHPSSNTAVAKVRTSSSVHESHSSVLADSETTITIPSMGITSAVDDTTVFTSNPAFSETRRIPTEPTFSLTPGFRETSTSEETTSITETSAVLYGVPTSATTEVSMTEIMSSNRTHIPDSDQSTMSPDIITEVITRLSSSSMMSESTQMTITTQKSSPGATAQSTLTLATTTAPLARTHSTVPPRFLHSEMTTLMSRSPENPSWKSSPFVEKTSSSSSLLSLPVTTSPSVSSTLPQSIPSSSFSVTSLLTPGMVKTTDTSTEPGTSLSPNLSGTSVEILAASEVTTDTEKIHPSSSMAVTNVGTTSSGHELYSSVSIHSEPSKATYPVGTPSSMAETSISTSMPANFETTGFEAEPFSHLTSGFRKTNMSLDTSSVTPTNTPSSPGSTHLLQSSKTDFTSSAKTSSPDWPPASQYTEIPVDIITPFNASPSITESTGITSFPESRFTMSVTESTHHLSTDLLPSAETISTGTVMPSLSEAMTSFATTGVPRAISGSGSPFSRTESGPGDATLSTIAESLPSSTPVPFSSSTFTTTDSSTIPALHEITSSSATPYRVDTSLGTESSTTEGRLVMVSTLDTSSQPGRTSSTPILDTRMTESVELGTVTSAYQVPSLSTRLTRTDGIMEHITKIPNEAAHRGTIRPVKGPQTSTSPASPKGLHTGGTKRMETTTTALKTTTTALKTTSRATLTTSVYTPTLGTLTPLNASRQMASTILTEMMITTPYVFPDVPETTSSLATSLGAETSTALPRTTPSVLNRESETTASLVSRSGAERSPVIQTLDVSSSEPDTTASWVIHPAETIPTVSKTTPNFFHSELDTVSSTATSHGADVSSAIPTNISPSELDALTPLVTISGTDTSTTFPTLTKSPHETETRTTWLTHPAETSSTIPRTIPNFSHHESDATPSIATSPGAETSSAIPIMTVSPGAEDLVTSQVTSSGTDRNMTIPTLTLSPGEPKTIASLVTHPEAQTSSAIPTSTISPAVSRLVTSMVTSLAAKTSTTNRALTNSPGEPATTVSLVTHPAQTSPTVPWTTSIFFHSKSDTTPSMTTSHGAESSSAVPTPTVSTEVPGVVTPLVTSSRAVISTTIPILTLSPGEPETTPSMATSHGEEASSAIPTPTVSPGVPGVVTSLVTSSRAVTSTTIPILTFSLGEPETTPSMATSHGTEAGSAVPTVLPEVPGMVTSLVASSRAVTSTTLPTLTLSPGEPETTPSMATSHGAEASSTVPTVSPEVPGVVTSLVTSSSGVNSTSIPTLILSPGELETTPSMATSHGAEASSAVPTPTVSPGVSGVVTPLVTSSRAVTSTTIPILTLSSSEPETTPSMATSHGVEASSAVLTVSPEVPGMVTSLVTSSRAVTSTTIPTLTISSDEPETTTSLVTHSEAKMISAIPTLAVSPTVQGLVTSLVTSSGSETSAFSNLTVASSQPETIDSWVAHPGTEASSVVPTLTVSTGEPFTNISLVTHPAESSSTLPRTTSRFSHSELDTMPSTVTSPEAESSSAISTTISPGIPGVLTSLVTSSGRDISATFPTVPESPHESEATASWVTHPAVTSTTVPRTTPNYSHSEPDTTPSIATSPGAEATSDFPTITVSPDVPDMVTSQVTSSGTDTSITIPTLTLSSGEPETTTSFITYSETHTSSAIPTLPVSPGASKMLTSLVISSGTDSTTTFPTLTETPYEPETTAIQLIHPAETNTMVPKTTPKFSHSKSDTTLPVAITSPGPEASSAVSTTTISPDMSDLVTSLVPSSGTDTSTTFPTLSETPYEPETTVTWLTHPAETSTTVSGTIPNFSHRGSDTAPSMVTSPGVDTRSGVPTTTIPPSIPGVVTSQVTSSATDTSTAIPTLTPSPGEPETTASSATHPGTQTGFTVPIRTVPSSEPDTMASWVTHPPQTSTPVSRTTSSFSHSSPDATPVMATSPRTEASSAVLTTISPGAPEMVTSQITSSGAATSTTVPTLTHSPGMPETTALLSTHPRTGTSKTFPASTVFPQVSETTASLTIRPGAETSTALPTQTTSSLFTLLVTGTSRVDLSPTASPGVSAKTAPLSTHPGTETSTMIPTSTLSLGLLETTGLLATSSSAETSTSTLTLTVSPAVSGLSSASITTDKPQTVTSWNTETSPSVTSVGPPEFSRTVTGTTMTLIPSEMPTPPKTSHGEGVSPTTILRTTMVEATNLATTGSSPTVAKTTTTFNTLAGSLFTPLTTPGMSTLASESVTSRTSYNHRSWISTTSSYNRRYWTPATSTPVTSTFSPGISTSSIPSSTAATVPFMVPFTLNFTITNLQYEEDMRHPGSRKFNATERELQGLLKPLFRNSSLEYLYSGCRLASLRPEKDSSAMAVDAICTHRPDPEDLGLDRERLYWELSNLTNGIQELGPYTLDRNSLYVNGFTHRSSMPTTSTPGTSTVDVGTSGTPSSSPSPTAAGPLLMPFTLNFTITNLQYEEDMRRTGSRKFNTMESVLQGLLKPLFKNTSVGPLYSGCRLTLLRPEKDGAATGVDAICTHRLDPKSPGLNREQLYWELSKLTNDIEELGPYTLDRNSLYVNGFTHQSSVSTTSTPGTSTVDLRTSGTPSSLSSPTIMAAGPLLVPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTTSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGLDREQLYWELSQLTNGIKELGPYTLDRNSLYVNGFTHWIPVPTSSTPGTSTVDLGSGTPSSLPSPTAAGPLLVPFTLNFTITNLQYEEDMHHPGSRKFNTTERVLQGLLGPMFKNTSVGLLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMRHPGSRKFNTTERVLQGLLKPLFKSTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATGMDAICSHRLDPKSPGLNREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTAGPLLVPFTLNFTITNLQYEEDMHRPGSRKFNTTERVLQGLLSPIFKNSSVGPLYSGCRLTSLRPEKDGAATGMDAVCLYHPNPKRPGLDREQLYWELSQLTHNITELGPYSLDRDSLYVNGFTHQNSVPTTSTPGTSTVYWATTGTPSSFPGHTEPGPLLIPFTFNFTITNLHYEENMQHPGSRKFNTTERVLQGLLKPLFKNTSVGPLYSGCRLTSLRPEKDGAATGMDAVCLYHPNPKRPGLDREQLYWELSQLTHNITELGPYSLDRDSLYVNGFTHQNSVPTTSTPGTSTVYWATTGTPSSFPGHTEPGPLLIPFTFNFTITNLHYEENMQHPGSRKFNTTERVLQGLLKPLFKNTSVGPLYSGCRLTLLRPEKHEAATGVDTICTHRVDPIGPGLDRERLYWELSQLTNSITELGPYTLDRDSLYVNGFNPRSSVPTTSTPGTSTVHLATSGTPSSLPGHTAPVPLLIPFTLNFTITNLHYEENMQHPGSRKFNTTERVLQGLLKPLFKNTSVGPLYSGCRLTLLRPEKHEAATGVDTICTHRVDPIGPGLXXEXLYWELSXLTXXIXELGPYTLDRXSLYVNGFTHXXSXPTTSTPGTSTVXXGTSGTPSSXPXXTSAGPLLVPFTLNFTITNLQYEEDMHHPGSRKFNTTERVLQGLLGPMFKNTSVGLLYSGCRLTLLRPEKNGAATGMDAICSHRLDPKSPGLDREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTAGPLLVPFTLNFTITNLQYEEDMHRPGSRKFNATERVLQGLLSPIFKNSSVGPLYSGCRLTSLRPEKDGAATGMDAVCLYHPNPKRPGLDREQLYWELSQLTHNITELGPYSLDRDSLYVNGFTHQSSMTTTRTPDTSTMHLATSRTPASLSGPTTASPLLVLFTINCTITNLQYEEDMRRTGSRKFNTMESVLQGLLKPLFKNTSVGPLYSGCRLTLLRPKKDGAATGVDAICTHRLDPKSPGLNREQLYWELSKLTNDIEELGPYTLDRNSLYVNGFTHQSSVSTTSTPGTSTVDLRTSGTPSSLSSPTIMXXXPLLXPFTXNXTITNLXXXXXMXXPGSRKFNTTERVLQGLLRPLFKNTSVSSLYSGCRLTLLRPEKDGAATRVDAACTYRPDPKSPGLDREQLYWELSQLTHSITELGPYTLDRVSLYVNGFNPRSSVPTTSTPGTSTVHLATSGTPSSLPGHTXXXPLLXPFTXNXTITNLXXXXXMXXPGSRKFNTTERVLQGLLKPLFRNSSLEYLYSGCRLASLRPEKDSSAMAVDAICTHRPDPEDLGLDRERLYWELSNLTNGIQELGPYTLDRNSLYVNGFTHRSSGLTTSTPWTSTVDLGTSGTPSPVPSPTTAGPLLVPFTLNFTITNLQYEEDMHRPGSRRFNTTERVLQGLLTPLFKNTSVGPLYSGCRLTLLRPEKQEAATGVDTICTHRVDPIGPGLDRERLYWELSQLTNSITELGPYTLDRDSLYVNGFNPWSSVPTTSTPGTSTVHLATSGTPSSLPGHTAPVPLLIPFTLNFTITDLHYEENMQHPGSRKFNTTERVLQGLLKPLFKSTSVGPLYSGCRLTLLRPEKHGAATGVDAICTLRLDPTGPGLDRERLYWELSQLTNSVTELGPYTLDRDSLYVNGFTHRSSVPTTSIPGTSAVHLETSGTPASLPGHTAPGPLLVPFTLNFTITNLQYEEDMRHPGSRKFSTTERVLQGLLKPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPTTASPLLVLFTINFTITNLRYEENMHHPGSRKFNTTERVLQGLLRPVFKNTSVGPLYSGCRLTTLRPKKDGAATKVDAICTYRPDPKSPGLDREQLYWELSQLTHSITELGPYTQDRDSLYVNGFTHRSSVPTTSIPGTSAVHLETSGTPASLPGHTAPGPLLVPFTLNFTITNLQYEEDMRHPGSRKFNTTERVLQGLLKPLFKSTSVGPLYSGCRLTLLRPEKRGAATGVDTICTHRLDPLNPGLDREQLYWELSKLTRGIIELGPYLLDRGSLYVNGFTHRTSVPTTSTPGTSTVDLGTSGTPFSLPSPAXXXPLLXPFTXNXTITNLXXXXXMXXPGSRKFNTTERVLQTLLGPMFKNTSVGLLYSGCRLTLLRSEKDGAATGVDAICTHRLDPKSPGVDREQLYWELSQLTNGIKELGPYTLDRNSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTSLRSEKDGAATGVDAICTHRVDPKSPGVDREQLYWELSQLTNGIKELGPYTLDRNSLYVNGFTHQTSAPNTSTPGTSTVXXGTSGTPSSXPXXTSAGPLLVPFTLNFTITNLQYEEDMHHPGSRKFNTTERVLQGLLGPMFKNTSVGLLYSGCRLTLLRPEKNGATTGMDAICTHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTERVLQGLLKPLFRNSSLEYLYSGCRLASLRPEKDSSAMAVDAICTHRPDPEDLGLDRERLYWELSNLTNGIQELGPYTLDRNSLYVNGFTHRSSMPTTSTPGTSTVDVGTSGTPSSSPSPTTAGPLLIPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTTSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTERVLQGLLRPVFKNTSVGPLYSGCRLTLLRPKKDGAATKVDAICTYRPDPKSPGLDREQLYWELSQLTHSITELGPYTQDRDSLYVNGFTHRSSVPTTSIPGTSAVHLETTGTPSSFPGHTEPGPLLIPFTFNFTITNLRYEENMQHPGSRKFNTTERVLQGLLTPLFKNTSVGPLYSGCRLTLLRPEKQEAATGVDTICTHRVDPIGPGLDRERLYWELSQLTNSITELGPYTLDRDSLYVDGFNPWSSVPTTSTPGTSTVHLATSGTPSPLPGHTAPVPLLIPFTLNFTITDLHYEENMQHPGSRKFNTTERVLQGLLKPLFKSTSVGPLYSGCRLTLLRPEKHGAATGVDAICTLRLDPTGPGLDRERLYWELSQLTNSITELGPYTLDRDSLYVNGFNPWSSVPTTSTPGTSTVHLATSGTPSSLPGHTTAGPLLVPFTLNFTITNLKYEEDMHCPGSRKFNTTERVLQSLHGPMFKNTSVGPLYSGCRLTLLRSEKDGAATGVDAICTHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTNSITELGPYTLDRDSLYVNGFTHRSSMPTTSIPGTSAVHLETSGTPASLPGHTAPGPLLVPFTLNFTITNLQYEEDMRHPGSRKFNTTERVLQGLLKPLFKSTSVGPLYSGCRLTLLRPEKRGAATGVDTICTHRLDPLNPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFHPRSSVPTTSTPGTSTVHLATSGTPSSLPGHTAPVPLLIPFTLNFTITNLHYEENMQHPGSRKFNTTERVLQGLLGPMFKNTSVGLLYSGCRLTLLRPEKNGAATGMDAICSHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHQNSVPTTSTPGTSTVYWATTGTPSSFPGHTEPGPLLIPFTFNFTITNLHYEENMQHPGSRKFNTTERVLQGLLTPLFKNTSVGPLYSGCRLTLLRPEKQEAATGVDTICTHRVDPIGPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHRSSVPTTSSPGTSTVHLATSGTPSSLPGHTAPVPLLIPFTLNFTITNLHYEENMQHPGSRKFNTTERVLQGLLKPLFKSTSVGPLYSGCRLTLLRPEKHGAATGVDAICTLRLDPTGPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHRTSVPTTSTPGTSTVHLATSGTPSSLPGHTAPVPLLIPFTLNFTITNLQYEEDMHRPGSRKFNTTERVLQGLLSPIFKNSSVGPLYSGCRLTSLRPEKDGAATGMDAVCLYHPNPKRPGLDREQLYCELSQLTHNITELGPYSLDRDSLYVNGFTHQNSVPTTSTPGTSTVYWATTGTPSSFPGHTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHWSSGLTTSTPWTSTVDLGTSGTPSPVPSPTTAGPLLVPFTLNFTITNLQYEEDMHRPGSRKFNATERVLQGLLSPIFKNTSVGPLYSGCRLTLLRPEKQEAATGVDTICTHRVDPIGPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHRSFGLTTSTPWTSTVDLGTSGTPSPVPSPTTAGPLLVPFTLNFTITNLQYEEDMHRPGSRKFNTTERVLQGLLTPLFRNTSVSSLYSGCRLTLLRPEKDGAATRVDAVCTHRPDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHWIPVPTSSTPGTSTVDLGSGTPSSLPSPTTAGPLLVPFTLNFTITNLQYGEDMGHPGSRKFNTTERVLQGLLGPIFKNTSVGPLYSGCRLTSLRSEKDGAATGVDAICIHHLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHQTFAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATRVDAVCTHRPDPKSPGLXXEXLYWELSXLTXXIXELGPYTLDRXSLYVNGFTHXXSXPTTSTPGTSTVXXGTSGTPSSXPXXTAPVPLLIPFTLNFTITNLHYEENMQHPGSRKFNTTERVLQGLLKPLFKSTSVGPLYSGCRLTLLRPEKHGAATGVDAICTLRLDPTGPGLDRERLYWELSQLTNSVTELGPYTLDRDSLYVNGFTQRSSVPTTSIPGTSAVHLETSGTPASLPGHTAPGPLLVPFTLNFTITNLQYEVDMRHPGSRKFNTTERVLQGLLKPLFKSTSVGPLYSGCRLTLLRPEKRGAATGVDTICTHRLDPLNPGLDREQLYWELSKLTRGIIELGPYLLDRGSLYVNGFTHRNFVPITSTPGTSTVHLGTSETPSSLPRPIVPGPLLVPFTLNFTITNLQYEEAMRHPGSRKFNTTERVLQGLLRPLFKNTSIGPLYSSCRLTLLRPEKDKAATRVDAICTHHPDPQSPGLNREQLYWELSQLTHGITELGPYTLDRDSLYVDGFTHWSPIPTTSTPGTSIVNLGTSGIPPSLPETTXXXPLLXPFTXNXTITNLXXXXXMXXPGSRKFNTTERVLQGLLKPLFKSTSVGPLYSGCRLTLLRPEKDGVATRVDAICTHRPDPKIPGLDRQQLYWELSQLTHSITELGPYTLDRDSLYVNGFTQRSSVPTTSTPGTFTVQPETSETPSSLPGPTATGPVLLPFTLNFTITNLQYEEDMHRPGSRKFNTTERVLQGLLMPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPTTASPLLVLFTINFTITNLRYEENMHHPGSRKFNTTERVLQGLLRPVFKNTSVGPLYSGCRLTLLRPKKDGAATKVDAICTYRPDPKSPGLDREQLYWELSQLTHSITELGPYTLDRDSLYVNGFTQRSSVPTTSIPGTPTVDLGTSGTPVSKPGPSAASPLLVLFTLNFTITNLRYEENMQHPGSRKFNTTERVLQGLLRSLFKSTSVGPLYSGCRLTLLRPEKDGTATGVDAICTHHPDPKSPRLDREQLYWELSQLTHNITELGHYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGSRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMKHLLSPLFQRSSLGARYTGCRVIALRSVKNGAETRVDLLCTYLQPLSGPGLPIKQVFHELSQQTHGITRLGPYSLDKDSLYLNGYNEPGLDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYSPNRNEPLTGNSDLPFWAVILIGLAGLLGLITCLICGVLVTTRRRKKEGEYNVQQQCPGYYQSHLDLEDLQ

SEQ ID NO: 4 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 5 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSIGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPGAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 6 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 7 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

(19) United States
(12) Patent Application Publication
(10) Pub. No.: US 2023/0086542 A1
(43) Pub. Date: Mar. 23, 2023

(54) COMPOSITIONS FOR ENDOMETRIOSIS ASSESSMENT HAVING IMPROVED SPECIFICITY

(71) Applicant: Aspira Women's Health Inc., Austin, TX (US)

(72) Inventors: Nitin BHARDWAJ, Austin, TX (US); Todd PAPPAS, Austin, TX (US); Herbert FRITSCHE, Austin, TX (US)

(73) Assignee: Aspira Women's Health Inc., Austin, TX (US)

(21) Appl. No.: 17/800,850

(22) PCT Filed: Feb. 18, 2021
(86) PCT No.: PCT/US21/18462

§ 371 Date: Aug. 18, 2022

Related U.S. Application Data
(60) Provisional application No. 62/978,471, filed on Feb. 19, 2020.
(60) Provisional application No. 63/146,100, filed on Feb. 5, 2021.

(51) Int. Cl. A61K 38/09; A61K 31/513; A61K 31/519; A61P 15/00; G01N 33/68; G01N 33/76

(52) U.S. Cl. CPC A61K 38/09; A61K 31/513; A61K 31/519; A61P 15/00; G01N 33/689; G01N 33/76; G01N 2800/364

(57) ABSTRACT

The present invention provides compositions and methods that provide a high degree of sensitivity and a high degree of specificity for the non-invasive assessment of endometriosis in women having a variety of endometriosis types (e.g., endometriosis, endometriotic cysts, endometrioma, or another benign condition of the endometrium) and at a variety of disease states (e.g., early and late stage).

### CROSS-REFERENCE TO RELATED APPLICATIONS

This application is an International PCT application, which claims priority to and the benefit of U.S. Provisional Patent Application Ser. No. 63/146,100 filed Feb. 5, 2021 and U.S. Provisional Patent Application Ser. No. 62/978,471 filed Feb. 19, 2020, the entire contents of all of which are hereby incorporated by reference herein.

### BACKGROUND OF THE INVENTION

Endometriosis is a debilitating gynecological disorder, which is difficult to diagnose and manage. Endometriosis is characterized by the implantation of benign endometrial tissue in locations outside the uterine cavity, including the pelvic peritoneum, ovaries, and bowel. Endometriosis affects 6-10% of women of reproductive age and 35-50% of women experiencing pain and/or unexplained infertility. Symptoms include dysmenorrhea, dyspareunia, chronic pelvic pain, and difficulty conceiving. Despite decades of research, there are no sufficiently sensitive and specific signs and symptoms nor blood tests for the clinical confirmation of endometriosis, which hampers prompt diagnosis and treatment. Laparoscopy is the gold standard diagnostic test for endometriosis but is expensive and carries surgical risks.

Several factors involved in the chronic inflammatory process of endometriosis, such as hormones, cytokines, chemokines, angiogenic factors, oxidative stress markers and others, have been implicated in the disease's pathogenesis and have been extensively studied, but most potential biomarkers have been discarded at the research stage and very few have been translated to clinical practice. Thus, it has not been possible to characterize the presence of endometriosis based on symptoms, clinical examination, imaging techniques or blood tests.

There remains an urgent need for improved diagnostic methods that not only have a high degree of sensitivity, but that also provide a high degree of specificity, which can be used to manage endometriosis treatment more effectively.

### SUMMARY OF THE INVENTION

The present invention provides compositions and methods that provide a high degree of sensitivity and a high degree of specificity for the pre-operative assessment of endometriosis in pre-menopausal women having a variety of endometriosis types (e.g., endometriosis, endometriotic cysts, endometrioma, or another benign condition of the endometrium) and at a variety of disease states (e.g., early and late stage).

In one aspect, the invention provides a panel for non-invasively characterizing endometriosis in a biological sample of a subject. In one aspect, the invention provides a panel for non-invasively characterizing endometriosis in a biological sample of a subject, the panel including polypeptide markers Apolipoprotein A1 (ApoA1), β2-microglobulin (B2M), Cancer Antigen 125 (CA125), Transferrin (TRF), Transthyretin (TT)/Prealbumin (PREA), Human Epididymis Protein 4 (HE4), Follicle Stimulating Hormone (FSH), and one of the following polypeptide markers: Chemokine 4 (CCL4, MIP-1β), Immunoglobin M (IgM), Luteinizing Hormone (LH), Macrophage Derived Chemokine (MDC, CCL22), and Progesterone (P4) or polynucleotides encoding such polypeptides.

In another aspect, the invention provides a panel for non-invasively characterizing endometriosis in a biological sample of a subject, the panel including polypeptide markers β2-microglobulin (B2M), Cancer Antigen 125 (CA125), Follicle Stimulating Hormone (FSH), Chemokine 4 (CCL4, MIP-1β), Immunoglobin M (IgM), Luteinizing Hormone (LH), Macrophage Derived Chemokine (MDC, CCL22), and Progesterone (P4) or polynucleotides encoding such polypeptides.

In another aspect, the invention provides a panel for non-invasively characterizing endometriosis in a biological sample of a subject, the panel including polypeptide markers Cancer Antigen 125 (CA125), Apolipoprotein A1 (ApoA1), Transferrin (TRF), β2-microglobulin (B2M), Follicle Stimulating Hormone (FSH), Human Epididymis Protein 4 (HE4), and Prealbumin (PREA) or polynucleotides encoding such polypeptides.

In another aspect, the invention provides a panel for non-invasively characterizing endometriosis in a biological sample of a subject, the panel including polypeptide markers Cancer Antigen 125 (CA125), Macrophage Derived Chemokine (MDC, CCL22), Progesterone (P4), EN-RAGE (S100A12), Immunoglobulin M (IgM), Chemokine 4 (CCL4, MIP-1β), β2-microglobulin (B2M), Follicle Stimulating Hormone (FSH), Luteinizing Hormone (LH), and Cystatin C (CST3) or polynucleotides encoding such polypeptides.

In another aspect, the invention provides a panel for non-invasively characterizing endometriosis in a biological sample of a subject, the panel including polypeptide markers Cancer Antigen 125 (CA125), Macrophage Derived Chemokine (MDC, CCL22), Progesterone (P4), and Apolipoprotein A1 (ApoA1) or polynucleotides encoding such polypeptides.

In another aspect, the invention provides a panel for non-invasively characterizing endometriosis in a biological sample of a subject, the panel including polypeptide markers Cancer Antigen 125 (CA125), Apolipoprotein A1 (ApoA1), Macrophage Derived Chemokine (MDC, CCL22), Progesterone (P4), EN-RAGE (S100A12), Immunoglobulin M (IgM), Chemokine 4 (CCL4, MIP-1β, HCC4), β2-microglobulin (B2M), Follicle Stimulating Hormone (FSH), Luteinizing Hormone (LH), and or polynucleotides encoding such polypeptides.

In another aspect, the invention provides a panel for non-invasively characterizing endometriosis in a biological sample of a subject, the panel including polypeptide markers Apolipoprotein A1 (ApoA1), β2-microglobulin (B2M), Cancer Antigen 125 (CA125), Transferrin (TRF), Transthyretin (TT)/Prealbumin (PREA), Human Epididymis Protein 4 (HE4), Follicle Stimulating Hormone (FSH) and one or more of the following polypeptide markers: Alpha 1 Microglobulin (A1M), Alpha Fetoprotein (AFP), Angiopoietin 2 (Ang-2), Apolipoprotein B (ApoB), Apolipoprotein E (ApoE), Cystatin C (CST3), CD 40 Antigen (CD40), Chromogranin A (CgA), Chemokine 4 (CCL4, MIP-1β), Clusterin (CLU, ApoJ), Eotaxin 1 (CCL11), Endostatin, EN-RAGE (S100A12), Fatty Acid Binding Protein (adipocyte)(FABP4), Fatty Acid Binding Protein (heart) (H-FABP, FABP3), Fas Ligand Receptor (Fas, FasR), Ferritin (FTL), Galectin 3 (Gal-3), Growth Hormone (GH, somatotropin, human growth hormone, HGH), Glutathione S Transferase alpha (GSTα), Human Chorionic Gonadotropin (hCG), Hepatocyte Growth Factor (HGF), Haptoglobin (Hp), Immunoglobulin E (IgE), Insulin like Growth Factor Binding Protein 4 (IGFBP4), Insulin like Growth Factor I (IGF-I), Immunoglobulin M (IgM), Interleukin 8 (IL-8, CXCL8), Interferon gamma Induced Protein 10 (IP-10, CXCL10), Interferon inducible T cell alpha chemoattractant (I-TAC, CXCL11), Kallikrein 5 (KLK5), Leptin (LEP), Luteinizing Hormone (LH), Monocyte Chemotactic Protein 1 (MCP-1, CCL2), Monocyte Chemotactic Protein 4 (MCP-4, CCL13), Macrophage Derived Chemokine (MDC, CCL22), Monokine Induced by Gamma Interferon (Mig, CXCL9), Macrophage Inflammatory Protein 1 alpha (MIP-1α, CCL3), Matrix Metalloproteinase 3 (MMP-3), Myoglobin (Mb), N terminal prohormone of brain natriuretic peptide (NT-proBNP), Osteoprotegerin (OPG, TNFRSF11B), Pulmonary and Activation Regulated Chemokine (PARC), Prostasin (PRSS8), Phosphoserine Aminotransferase (PSAT), Stem Cell Factor (SCF), Thymus Expressed Chemokine (TECK), Trefoil Factor 3 (TFF3), Tumor Necrosis Factor alpha (TNFα, TNF), Tumor Necrosis Factor Receptor 1 (TNFR1), Tumor Necrosis Factor Receptor 2 (TNFR2), Tissue type Plasminogen activator (tPA/PLAT), Urokinase type Plasminogen Activator (uPA), Urokinase type Plasminogen Activator Receptor (uPAR, CD87), Vascular Endothelial Growth Factor (VEGF), von Willebrand Factor (VWF), YKL-40 (CHI3L1) or polynucleotides encoding such polypeptides.

In various embodiments of the previous aspects, or any other aspect of the invention delineated herein, the markers are bound to a capture molecule. In various embodiments of the previous aspects, or any other aspect of the invention delineated herein, the capture molecule is bound to a substrate.

In one aspect, the invention provides a panel of capture molecules, where each capture molecule binds a polypeptide biomarker of any one of the previous aspects, or any other aspect of the invention delineated herein.

In various embodiments of the previous aspects, or any other aspect of the invention delineated herein, the capture molecule is an antibody. In various embodiments of the previous aspects, or any other aspect of the invention delineated herein, the capture molecule is a polynucleotide.

In another aspect, the invention provides a method of treating a selected subject, the method involving administering to the subject a gonadotropin-releasing hormone (GnRH) antagonist or a GnRH agonist. In one aspect, the invention provides a method of treating a selected subject, the method involving administering to the subject a gonadotropin-releasing hormone (GnRH) antagonist or a GnRH agonist, where the subject is selected by characterizing a biological sample of the subject as having an alteration in the level of a biomarker relative to a reference, where the biomarker is selected from the group of Apolipoprotein A1 (ApoA1), β2-microglobulin (B2M), Cancer Antigen 125 (CA125), Transferrin (TRF), Transthyretin (TT)/Prealbumin (PREA), Human Epididymis Protein 4 (HE4), Follicle Stimulating Hormone (FSH), and one or more of the following polypeptide markers: Chemokine 4 (CCL4, MIP-1β), Immunoglobin M (IgM), Luteinizing Hormone (LH), Macrophage Derived Chemokine (MDC, CCL22), and Progesterone (P4).

In another aspect, the invention provides a method of treating a selected subject, the method involving administering to the subject a gonadotropin-releasing hormone (GnRH) antagonist or a GnRH agonist, where the subject is selected by characterizing a biological sample of the subject as having an alteration in the level of a biomarker relative to a reference, where the biomarker is selected from the group of β2-microglobulin (B2M), Cancer Antigen 125 (CA125), Follicle Stimulating Hormone (FSH), Chemokine 4 (CCL4, MIP-1β), Immunoglobin M (IgM), Luteinizing Hormone (LH), Macrophage Derived Chemokine (MDC, CCL22), and Progesterone (P4).

In another aspect, the invention provides a method of treating a selected subject, the method involving administering to the subject a gonadotropin-releasing hormone (GnRH) antagonist or a GnRH agonist, where the subject is selected by characterizing a biological sample of the subject as having an alteration in the level of a biomarker relative to a reference, where the biomarker is selected from the group of Apolipoprotein A1 (ApoA1), β2-microglobulin (B2M), Cancer Antigen 125 (CA125), Transferrin (TRF), Transthyretin (TT)/Prealbumin (PREA), Human Epididymis Protein 4 (HE4), Follicle Stimulating Hormone (FSH) and one or more of the following polypeptide markers: Alpha 1 Microglobulin (A1M), Alpha Fetoprotein (AFP), Angiopoietin 2 (Ang-2), Apolipoprotein B (ApoB), Apolipoprotein E (ApoE), Cystatin C (CST3), CD 40 Antigen (CD40), Chromogranin A (CgA), Chemokine 4 (CCL4, MIP-1β), Clusterin (CLU, ApoJ), Eotaxin 1 (CCL11), Endostatin, EN-RAGE (S100A12), Fatty Acid Binding Protein (adipocyte)(FABP4), Fatty Acid Binding Protein (heart) (H-FABP, FABP3), Fas Ligand Receptor (Fas, FasR), Ferritin (FTL), Galectin 3 (Gal-3), Growth Hormone (GH, somatotropin, human growth hormone, HGH), Glutathione S Transferase alpha (GSTα), Human Chorionic Gonadotropin (hCG), Hepatocyte Growth Factor (HGF), Haptoglobin (Hp), Immunoglobulin E (IgE), Insulin like Growth Factor Binding Protein 4 (IGFBP4), Insulin like Growth Factor I (IGF-I), Immunoglobulin M (IgM), Interleukin 8 (IL-8, CXCL8), Interferon gamma Induced Protein 10 (IP-10, CXCL10), Interferon inducible T cell alpha chemoattractant (I-TAC, CXCL11), Kallikrein 5 (KLK5), Leptin (LEP), Luteinizing Hormone (LH), Monocyte Chemotactic Protein 1 (MCP-1, CCL2), Monocyte Chemotactic Protein 4 (MCP-4, CCL13), Macrophage Derived Chemokine (MDC, CCL22), Monokine Induced by Gamma Interferon (Mig, CXCL9), Macrophage Inflammatory Protein 1 alpha (MIP-1α, CCL3), Matrix Metalloproteinase 3 (MMP-3), Myoglobin (Mb), N terminal prohormone of brain natriuretic peptide (NT-proBNP), Osteoprotegerin (OPG, TNFRSF111B), Pulmonary and Activation Regulated Chemokine (PARC), Progesterone (P4), Prostasin (PRSS8), Phosphoserine Aminotransferase (PSAT), Stem Cell Factor (SCF), Thymus Expressed Chemokine (TECK), Trefoil Factor 3 (TFF3), Tumor Necrosis Factor alpha (TNFα, TNF), Tumor Necrosis Factor Receptor 1 (TNFR1), Tumor Necrosis Factor Receptor 2 (TNFR2), Tissue type Plasminogen activator (tPA/PLAT), Urokinase type Plasminogen Activator (uPA), Urokinase type Plasminogen Activator Receptor (uPAR, CD87), Vascular Endothelial Growth Factor (VEGF), von Willebrand Factor (VWF), YKL-40 (CHI3L1).

In another aspect, the invention provides a method of treating a selected subject, the method involves administering to the subject a gonadotropin-releasing hormone (GnRH) antagonist or a GnRH agonist, where the subject is selected by characterizing a biological sample of the subject as having an alteration in the level of a biomarker relative to a reference, and the biomarker is selected from the group of Cancer Antigen 125 (CA125), Macrophage Derived Chemokine (MDC, CCL22), Progesterone (P4), and Apolipoprotein A1 (ApoA1).

In another aspect, the invention provides method of treating a selected subject, the method involves administering to the subject a gonadotropin-releasing hormone (GnRH) antagonist or a GnRH agonist, where the subject is selected by characterizing a biological sample of the subject as having an alteration in the level of a biomarker relative to a reference, and the biomarker is selected from the group consisting of Cancer Antigen 125 (CA125), Apolipoprotein A1 (ApoA1), Transferrin (TRF), β2-microglobulin (B2M), Follicle Stimulating Hormone (FSH), Human Epididymis Protein 4 (HE4), and Prealbumin (PREA) or polynucleotides encoding such polypeptides.

In another aspect, the invention provides a method of treating a selected subject, the method involves administering to the subject a gonadotropin-releasing hormone (GnRH) antagonist or a GnRH agonist, where the subject is selected by characterizing a biological sample of the subject as having an alteration in the level of a biomarker relative to a reference, and the biomarker is selected from the group consisting of Cancer Antigen 125 (CA125), Macrophage Derived Chemokine (MDC, CCL22), Progesterone (P4), EN-RAGE (S100A12), Immunoglobulin M (IgM), Chemokine 4 (CCL4, MIP-1β), β2-microglobulin (B2M), Follicle Stimulating Hormone (FSH), Luteinizing Hormone (LH), and Cystatin C (CST3) or polynucleotides encoding such polypeptides.

In another aspect, the invention provides a method of treating a selected subject, the method involving administering to the subject a gonadotropin-releasing hormone (GnRH) antagonist or a GnRH agonist, where the subject is selected by characterizing a biological sample of the subject as having an alteration in the level of a biomarker relative to a reference, and the biomarker is selected from the group consisting of Cancer Antigen 125 (CA125), Apolipoprotein A1 (ApoA1), Macrophage Derived Chemokine (MDC, CCL22), Progesterone (P4), EN-RAGE (S100A12), Immunoglobulin M (IgM), Chemokine 4 (CCL4, MIP-1β, HCC4), β2-microglobulin (B2M), Follicle Stimulating Hormone (FSH), Luteinizing Hormone (LH), and or polynucleotides encoding such polypeptides.

In various embodiments of the previous aspects, or any other aspect of the invention delineated herein, the method involves characterizing the age of the subject. In various embodiments of the previous aspects, or any other aspect of the invention delineated herein, the method involves characterizing the subject as pre-menopausal or post-menopausal. In various embodiments of the previous aspects, or any other aspect of the invention delineated herein, the GnRH antagonist is elagolix, abarelix, cetrorelix, degarelix, ganirelix, or relugolix. In various embodiments of the previous aspects, or any other aspect of the invention delineated herein, the GnRH antagonist is elagolix. In various embodiments of the previous aspects, or any other aspect of the invention delineated herein, the GnRH agonist is goserelin, leuprolide, nafarelin, buserelin, gonadorelin, histrelin, or triptorelin.

In various embodiments of the previous aspects, or any other aspect of the invention delineated herein, an increase in the level of one or more of said markers distinguishes endometriosis from non-endometriosis. In various embodiments of the previous aspects, or any other aspect of the invention delineated herein, a decrease in the level of one or more of said markers distinguishes endometriosis from non-endometriosis. In various embodiments of the previous aspects, or any other aspect of the invention delineated herein, the reference is a corresponding biological sample derived from a healthy subject. In various embodiments of the previous aspects, or any other aspect of the invention delineated herein, the reference is derived from the same subject at an earlier point in time.

In various embodiments of the previous aspects, or any other aspect of the invention delineated herein, the characterizing step is an immunoassay or affinity capture. In various embodiments of the previous aspects, or any other aspect of the invention delineated herein, the immunoassay includes affinity capture assay, immunometric assay, heterogeneous chemiluminscence immunometric assay, homogeneous chemiluminscence immunometric assay, ELISA, western blotting, radioimmunoassay, magnetic immunoassay, real-time immunoquantitative PCR (iqPCR) and SERS label free assay. In various embodiments of the previous aspects, or any other aspect of the invention delineated herein, the method is carried out in a plate, chip, beads, microfluidic platform, membrane, planar microarray, or suspension array. In various embodiments of the previous aspects, or any other aspect of the invention delineated herein, the method detects a CA125 glycoform.

In one aspect, the invention provides a method for determining the marker profile of a biological sample. In another aspect, the invention provides a method for determining the marker profile of a biological sample, the method involving quantifying the levels of a marker of Table 1 in the sample.

In various embodiments of the previous aspects, or any other aspect of the invention delineated herein, the biological sample is a biological fluid selected from the group consisting of blood, blood serum, and plasma.

In another aspect, the invention provides a kit for detecting endometriosis in a biological sample. In some embodiments, the kit includes a set of capture molecules each of which specifically binds a marker of Table 1. In some embodiments, the kit includes a set of capture molecules each of which specifically binds a marker of the previous aspects or any other aspect of the invention delineated herein.

As described in detail herein, any method known in the art can be used to measure a panel of biomarkers. In aspects of the invention, the panel of biomarkers are measured using any immunoassay well known in the art. In embodiments, the immunoassay can be, but is not limited to, ELISA, western blotting, and radioimmunoassay.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

A “biomarker” or “marker” as used herein generally refers to a protein, nucleic acid molecule, clinical indicator, or other analyte that is associated with a disease. In one embodiment, a marker of endometriosis is differentially present in a biological sample obtained from a subject having or at risk of developing endometriosis relative to a reference. A marker is differentially present if the mean or median level of the biomarker present in the sample is statistically different from the level present in a reference. A reference level may be, for example, the level present in a sample obtained from a healthy control subject or the level obtained from the subject at an earlier timepoint, i.e., prior to treatment. Common tests for statistical significance include, among others, t-test, ANOVA, Kruskal-Wallis, Wilcoxon, Mann-Whitney and odds ratio. Biomarkers, alone or in combination, provide measures of relative likelihood that a subject belongs to a phenotypic status of interest. The differential presence of a marker of the invention in a subject sample can be useful in characterizing the subject as having or at risk of developing endometriosis, for determining the prognosis of the subject, for evaluating therapeutic efficacy, or for selecting a treatment regimen (e.g., selecting that the subject be evaluated and/or treated by a surgeon that specializes in endometriosis).

In one embodiment, markers useful in the panels of the invention include, for example, ApoA1, B2M, TRF, TT/PREA, CA125, HE4, and FSH, as well as the nucleic acid molecules encoding such proteins. In another embodiment, markers useful in the panels of the invention include, for example, B2M, CA125, FSH, CCL4/MIP-1l, IgM, LH, MDC/CCL22, and P4. In another embodiment, markers useful in the panels of the invention include, for example, CA125, ApoA1, TRF, B2M, FSH, HE4, and PREA. In another embodiment, markers useful in the panels of the invention include, for example, CA125, MDC, P4, EN-RAGE, IgM, HCC4, B2M, FSH, LH, and CST3. In some embodiments, markers useful in the panels of the invention include CA125, MDC, P4, and ApoA1. In another embodiment, any of the panels of the invention provided herein include age as a further biomarker. Fragments useful in the methods of the invention are sufficient to bind an antibody that specifically recognizes the protein from which the fragment is derived. The invention includes markers that are substantially identical to the following sequences. Preferably, such a sequence is at least 85%, 90%, 95% or even 99% identical at the amino acid level or nucleic acid to the sequence used for comparison.

By “Follicle-stimulating hormone (FSH) polypeptide” is meant a polypeptide or fragment thereof having at least about 85% amino acid identity to UniProt Accession No. P01225, and which binds an antibody that specifically binds an FSH polypeptide.

By “Human Epididymis Protein 4 (HE4) polypeptide” is meant a polypeptide or fragment thereof having at least about 85% amino acid identity to UniProt Accession No. Q14508, and which binds an antibody that specifically binds an HE4 polypeptide.

By “Cancer Antigen 125 (CA 125) polypeptide” is meant a polypeptide or fragment thereof having at least about 85% amino acid identity to UniProt Accession No. Q8WX17, and which binds an antibody that specifically binds a CA125 polypeptide.

By “Transthyretin (Prealbumin) (TT/PREA) polypeptide” is meant a polypeptide or fragment thereof having at least about 85% amino acid identity to UniProt Accession No. P02766, and which binds an antibody that specifically binds a transthyretin polypeptide.

By “Transferrin (TRF) polypeptide” is meant a polypeptide or fragment thereof having at least about 85% amino acid identity to UniProt Accession No. P02787, and which binds an antibody that specifically binds a transferrin polypeptide.

By “Apolipoprotein A1 (ApoA1) polypeptide” is meant a polypeptide or fragment thereof having at least about 85% amino acid identity to UniProt Accession No. P02647, and which binds an antibody that specifically binds an ApoA1 polypeptide.

By “β-2 microglobulin (B2M) polypeptide” is meant a polypeptide or fragment thereof having at least about 85% amino acid identity to UniProt Accession No. P61769, and which binds an antibody that specifically binds a B2M polypeptide.

By “Chemokine 4 (CCL4, MIP-1β) polypeptide” is meant a polypeptide or fragment thereof having at least about 85% amino acid identity to UniProt Accession No. P13236, and which binds an antibody that specifically binds a chemokine 4 polypeptide.

By “Immunoglobulin M (IgM) polypeptide” is meant a polypeptide or fragment thereof having at least about 85% amino acid identity to UniProt Accession No. P0DOX6 (Immunoglobulin mu heavy chain), and which binds an antibody that specifically binds an IgM polypeptide.

By “Luteinizing Hormone (LH) polypeptide” is meant a polypeptide or fragment thereof having at least about 85% amino acid identity to UniProt Accession No. P01229, and which binds an antibody that specifically binds a luteinizing hormone polypeptide.

By “Macrophage Derived Chemokine (MDC, CCL22)” is meant a polypeptide or fragment thereof having at least about 85% amino acid identity to UniProt Accession No. O00626, and which binds an antibody that specifically binds a macrophage derived chemokine polypeptide.

By “Progesterone (P4)” is meant a sex hormone involved in the regulation of the menstrual cycle and pregnancy, and which binds to an antibody that specifically binds progesterone.

By “agent” is meant any small molecule chemical compound, antibody, nucleic acid molecule, or polypeptide, or fragments thereof.

By “alteration” or “change” is meant an increase or decrease. An alteration may be by as little as 1%, 2%, 3%, 4%, 5%, 10%, 20%, 30%, or by 40%, 50%, 60%, or even by as much as 70%, 75%, 80%, 90%, or 100%.

By “biologic sample” is meant any tissue, cell, fluid, or other material derived from an organism.

By “capture reagent” is meant a reagent that specifically binds a nucleic acid molecule or polypeptide to select or isolate the nucleic acid molecule or polypeptide.

As used herein, the terms “determining”, “assessing”, “assaying”, “measuring” and “detecting” refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

The term “subject” or “patient” refers to an animal which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

By “marker profile” is meant a characterization of the expression or expression level of two or more polypeptides or polynucleotides.

By “endometriosis” is meant a gynecological disorder characterized by the implantation of benign endometrial tissue in locations outside the uterine cavity, including the pelvic peritoneum, ovaries, and bowel.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “reference” is meant a standard of comparison. For example, the marker level(s) present in a patient sample may be compared to the level of the marker in a corresponding healthy cell or tissue or in a diseased cell or tissue (e.g., a cell or tissue derived from a subject having endometriosis). In particular embodiments, the polypeptide level present in a patient sample may be compared to the level of said polypeptide present in a corresponding sample obtained at an earlier time point (i.e., prior to treatment), to a cell or tissue of another benign condition. As used herein, the term “sample” includes a biologic sample such as any tissue, cell, fluid, or other material derived from an organism.

By “specifically binds” is meant a compound (e.g., antibody) that recognizes and binds a molecule (e.g., polypeptide), but which does not substantially recognize and bind other molecules in a sample, for example, a biological sample.

The accuracy of a diagnostic test can be characterized using any method well known in the art, including, but not limited to, a Receiver Operating Characteristic curve (“ROC curve”). An ROC curve shows the relationship between sensitivity and specificity. Sensitivity is the percentage of true positives that are predicted by a test to be positive, while specificity is the percentage of true negatives that are predicted by a test to be negative. An ROC is a plot of the true positive rate against the false positive rate for the different possible cutpoints of a diagnostic test. Thus, an increase in sensitivity will be accompanied by a decrease in specificity. The closer the curve follows the left axis and then the top edge of the ROC space, the more accurate the test. Conversely, the closer the curve comes to the 45-degree diagonal of the ROC graph, the less accurate the test. The area under the ROC is a measure of test accuracy. The accuracy of the test depends on how well the test separates the group being tested into those with and without the disease in question. An area under the curve (referred to as “AUC”) of 1 represents a perfect test. In embodiments, biomarkers and diagnostic methods of the present invention have an AUC greater than 0.50, greater than 0.60, greater than 0.70, greater than 0.80, or greater than 0.9.

Other useful measures of the utility of a test are positive predictive value (“PPV”) and negative predictive value (“NPV”). PPV is the percentage of actual positives who test as positive. NPV is the percentage of actual negatives that test as negative.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Any compounds, compositions, or methods provided herein can be combined with one or more of any of the other compositions and methods provided herein.

As used herein, the singular forms “a”, “an”, and “the” include plural forms unless the context clearly dictates otherwise. Thus, for example, reference to “a biomarker” includes reference to more than one biomarker.

Unless specifically stated or obvious from context, as used herein, the term “or” is understood to be inclusive.

The term “including” is used herein to mean, and is used interchangeably with, the phrase “including but not limited to.”

As used herein, the terms “comprises,” “comprising,” “containing,” “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### BRIEF DESCRIPTION OF THE DRAWINGS

FIG. 1 is a table of multiplication factors used for marker value normalization in the analysis of the biomarkers of the invention.

FIG. 2 is a graphic depiction showing that A1M is decreased in subjects with endometriosis compared to those with other benign conditions.

FIG. 3 is a graphic depiction showing that AFP is decreased in subjects with endometriosis compared to those with other benign conditions.

FIG. 4 is a graphic depiction showing that Ang-2 is decreased in subjects with endometriosis compared to those with other benign conditions.

FIG. 5 is a graphic depiction showing that ApoB is decreased in subjects with endometriosis compared to those with other benign conditions.

FIG. 6 is a graphic depiction showing that ApoE is decreased in subjects with endometriosis compared to those with other benign conditions.

FIG. 7 is a graphic depiction showing that B2M is decreased in subjects with endometriosis compared to those with other benign conditions.

FIG. 8 is a graphic depiction showing that CST3 is decreased in subjects with endometriosis compared to those with other benign conditions.

FIG. 9 is a graphic depiction showing that CA125 is increased in subjects with endometriosis compared to those with other benign conditions.

FIG. 10 is a graphic depiction showing that CD40 is decreased in subjects with endometriosis compared to those with other benign conditions.

FIG. 11 is a graphic depiction showing that CgA is decreased in subjects with endometriosis compared to those with other benign conditions.

FIG. 12 is a graphic depiction showing that CCL4 is decreased in subjects with endometriosis compared to those with other benign conditions.

FIG. 13 is a graphic depiction showing that CLU is decreased in subjects with endometriosis compared to those with other benign conditions.

FIG. 14 is a graphic depiction showing that CCL11 is decreased in subjects with endometriosis compared to those with other benign conditions.

FIG. 15 is a graphic depiction showing that endostatin is decreased in subjects with endometriosis compared to those with other benign conditions.

FIG. 16 is a graphic depiction showing that EN-RAGE is increased in subjects with endometriosis compared to those with other benign conditions.

FIG. 17 is a graphic depiction showing that FABP4 is decreased in subjects with endometriosis compared to those with other benign conditions.

FIG. 18 is a graphic depiction showing that H-FABP is decreased in subjects with endometriosis compared to those with other benign conditions.

FIG. 19 is a graphic depiction showing that FasR is decreased in subjects with endometriosis compared to those with other benign conditions.

FIG. 20 is a graphic depiction showing that FTL is decreased in subjects with endometriosis compared to those with other benign conditions.

FIG. 21 is a graphic depiction showing that FSH is decreased in subjects with endometriosis compared to those with other benign conditions.

FIG. 22 is a graphic depiction showing that Gal-3 is decreased in subjects with endometriosis compared to those with other benign conditions.

FIG. 23 is a graphic depiction showing that GH is increased in subjects with endometriosis compared to those with other benign conditions.

FIG. 24 is a graphic depiction showing that GSTα is decreased in subjects with endometriosis compared to those with other benign conditions.

FIG. 25 is a graphic depiction showing that hCG is decreased in subjects with endometriosis compared to those with other benign conditions.

FIG. 26 is a graphic depiction showing that HGF is decreased in subjects with endometriosis compared to those with other benign conditions.

FIG. 27 is a graphic depiction showing that Hp is decreased in subjects with endometriosis compared to those with other benign conditions.

FIG. 28 is a graphic depiction showing that IgE is decreased in subjects with endometriosis compared to those with other benign conditions.

FIG. 29 is a graphic depiction showing that IGFBP4 is decreased in subjects with endometriosis compared to those with other benign conditions.

FIG. 30 is a graphic depiction showing that IGF-I is increased in subjects with endometriosis compared to those with other benign conditions.

FIG. 31 is a graphic depiction showing that IgM is increased in subjects with endometriosis compared to those with other benign conditions.

FIG. 32 is a graphic depiction showing that IL-8 is decreased in subjects with endometriosis compared to those with other benign conditions.

FIG. 33 is a graphic depiction showing that IP-10 is decreased in subjects with endometriosis compared to those with other benign conditions.

FIG. 34 is a graphic depiction showing that I-TAC is decreased in subjects with endometriosis compared to those with other benign conditions.

FIG. 35 is a graphic depiction showing that KLK5 is increased in subjects with endometriosis compared to those with other benign conditions.

FIG. 36 is a graphic depiction showing that LEP is decreased in subjects with endometriosis compared to those with other benign conditions.

FIG. 37 is a graphic depiction showing that LH is decreased in subjects with endometriosis compared to those with other benign conditions.

FIG. 38 is a graphic depiction showing that MCP-1 is decreased in subjects with endometriosis compared to those with other benign conditions.

FIG. 39 is a graphic depiction showing that MCP-4 is decreased in subjects with endometriosis compared to those with other benign conditions.

FIG. 40 is a graphic depiction showing that MDC is decreased in subjects with endometriosis compared to those with other benign conditions.

FIG. 41 is a graphic depiction showing that Mig is decreased in subjects with endometriosis compared to those with other benign conditions.

FIG. 42 is a graphic depiction showing that MIP-1α is decreased in subjects with endometriosis compared to those with other benign conditions.

FIG. 43 is a graphic depiction showing that MMP-3 is decreased in subjects with endometriosis compared to those with other benign conditions.

FIG. 44 is a graphic depiction showing that Mb is decreased in subjects with endometriosis compared to those with other benign conditions.

FIG. 45 is a graphic depiction showing that NT-proBNP is decreased in subjects with endometriosis compared to those with other benign conditions.

FIG. 46 is a graphic depiction showing that OPG is decreased in subjects with endometriosis compared to those with other benign conditions.

FIG. 47 is a graphic depiction showing that PARC is decreased in subjects with endometriosis compared to those with other benign conditions.

FIG. 48 is a graphic depiction showing that P4 is increased in subjects with endometriosis compared to those with other benign conditions.

FIG. 49 is a graphic depiction showing that PRSS8 is decreased in subjects with endometriosis compared to those with other benign conditions.

FIG. 50 is a graphic depiction showing that PSAT is decreased in subjects with endometriosis compared to those with other benign conditions.

FIG. 51 is a graphic depiction showing that SCF is decreased in subjects with endometriosis compared to those with other benign conditions.

FIG. 52 is a graphic depiction showing that TECK is decreased in subjects with endometriosis compared to those with other benign conditions.

FIG. 53 is a graphic depiction showing that TFF3 is decreased in subjects with endometriosis compared to those with other benign conditions.

FIG. 54 is a graphic depiction showing that TNFα is decreased in subjects with endometriosis compared to those with other benign conditions.

FIG. 55 is a graphic depiction showing that TNFR1 is decreased in subjects with endometriosis compared to those with other benign conditions.

FIG. 56 is a graphic depiction showing that TNFR2 is decreased in subjects with endometriosis compared to those with other benign conditions.

FIG. 57 is a graphic depiction showing that tPA is decreased in subjects with endometriosis compared to those with other benign conditions.

FIG. 58 is a graphic depiction showing that uPA is increased in subjects with endometriosis compared to those with other benign conditions.

FIG. 59 is a graphic depiction showing that uPAR is decreased in subjects with endometriosis compared to those with other benign conditions.

FIG. 60 is a graphic depiction showing that VEGF is decreased in subjects with endometriosis compared to those with other benign conditions.

FIG. 61 is a graphic depiction showing that VWF is decreased in subjects with endometriosis compared to those with other benign conditions.

FIG. 62 is a graphic depiction showing that CHI3L1 is decreased in subjects with endometriosis compared to those with other benign conditions.

FIG. 63 provides exemplary sequences of selected polypeptides useful in the methods of the invention.

FIG. 64 shows classifier and performance reproducibility depicted in four histograms showing frequency versus sensitivity and selectivity (left and right panels, respectively) for 511 data points using random forest (RF) and support vector machine (SVM) methods (top and bottom panels, respectively).

FIG. 65 shows the features distribution for the entire dataset, including FSH, HE4, TRF, B2M, ApoA1, CA125, TT, menopausal status, and age.

FIG. 66 shows the features distribution for equally sized sets, including FSH, HE4, TRF, B2M, ApoA1, CA125, TT, menopausal status, and age.

FIG. 67 shows the features distribution for pre-menopausal status sets, including FSH, HE4, TRF, B2M, ApoA1, CA125, TT, menopausal status, and age.

FIG. 68 shows classifier and performance reproducibility depicted in four histograms showing frequency versus sensitivity and selectivity (left and right panels, respectively) for data points using random forest (RF) and support vector machine (SVM) methods (top and bottom panels, respectively).

FIG. 69 shows the performance comparison from two operators (earlier and rerun) and the data set using random forest (RF) and support vector machine (SVM) methods.

FIG. 70 shows the specificity and sensitivity performance results on the data using different classifiers, including RF, SVM, Adaboost, xgbDART, MARS, and MARS*.

FIG. 71 is a plot of specificity versus sensitivity and a corresponding table highlighting the top results for specificity and sensitivity using varying preprocessing and classifiers.

FIG. 72 repeats the plot of FIG. 71 and shows a plot of true positive fraction versus false positive fraction for the top result, YeoJohnson, naïve_bayes.

FIG. 73 provides a schematic presenting how perceptrons are linked into a deep neural network (deep learning) and representative results obtained using a deep neural network to analyze the Correlogic data set. The results presented in FIG. 73 were obtained using the biomarkers listed in FIG. 76: MDC, Age, Progesterone, CA125, HCC4, FSH, B2M, EN RAGE, Cystatin C, LH, IGM, and prealbumin.

FIG. 74 provides plots and tables summarizing sensitivity and specificity obtained for the Bristow and 522 data sets.

FIG. 75 provides plots and tables summarizing sensitivity and specificity obtained for the Correlogic data set.

FIG. 76 provides a bar graph summarizing feature importance in a deep neural network of biomarkers Age (Feature 0), HCC4 (Feature 1), CA125 (Feature 2), ENRAGE (Feature 3), B2M (Feature 4), FSH (Feature 5), IGM (Feature 6), PREA (Feature 7), MDC (Feature 8), Progesterone (Feature 9), LH (Feature 10), and Cystain C (Feature 11) in both endometriosis positive and negative patients.

FIGS. 77A and 77B are flow charts depicting machine-language (ML) classifiers. FIG. 77A is a flow chart describing data combination and filtering from two separate datasets for use in development of classifier models. FIG. 77B is a flow chart presenting a representative workflow of model development and a testing paradigm.

FIG. 78 is a plot graph showing average specificity and sensitivity for a selected set of pre-processing techniques and classification algorithm combinations in Study I.

### DETAILED DESCRIPTION OF THE INVENTION

The invention comprises panels of biomarkers and the use of such panels for characterizing endometriosis.

The invention is based, at least in part, on the discovery of biomarkers useful for the non-invasive characterization of endometriosis. In some embodiments, a panel of the invention comprises one or more of the following polypeptide biomarkers B2M, ApoA1, transferrin, transthyretin, CA125, FSH, and/or HE4 or polynucleotides encoding such biomarkers. In other embodiments, the panels of the invention comprise B2M, ApoA1, transferrin, transthyretin, CA125, FSH, and/or HE4 or polynucleotides encoding such biomarkers and any one or more of the following polypeptide markers

Alpha Fetoprotein (AFP),

Angiopoietin 2 (Ang-2),

Apolipoprotein A1 (ApoA1),

Apolipoprotein B (ApoB),

Apolipoprotein E (ApoE),

β2-Microglobulin (B2M),

Cystatin C (CST3),

Cancer Antigen 125 (CA125, MUC16),

CD 40 Antigen (CD40),

Chromogranin A (CgA),

Chemokine 4 (CCL4, MIP-13),

Clusterin (CLU, ApoJ),

Eotaxin 1 (CCL11),

Endostatin,

EN-RAGE (S100A12),

Fatty Acid Binding Protein (adipocyte)(FABP4),

Fatty Acid Binding Protein (heart) (H-FABP, FABP3),

Fas Ligand Receptor (Fas, FasR),

Ferritin (FTL),

Follicle Stimulating Hormone (FSH),

Galectin 3 (Gal-3),

Growth Hormone (GH, somatotropin, human growth hormone, HGH),

Glutathione S Transferase alpha (GSTα), Human Epididymis Protein 4 (HE4, WFDC2),

Human Chorionic Gonadotropin (hCG),

Hepatocyte Growth Factor (HGF),

Haptoglobin (Hp),

Immunoglobulin E (IgE),

Insulin like Growth Factor Binding Protein 4 (IGFBP4),

Insulin like Growth Factor I (IGF-I),

Immunoglobulin M (IgM),

Interleukin 8 (IL-8, CXCL8),

Interferon gamma Induced Protein 10 (IP-10, CXCL10),

Interferon inducible T cell alpha chemoattractant (I-TAC, CXCL11),

Kallikrein 5 (KLK5) polypeptide,

Leptin (LEP) polypeptide,

Luteinizing Hormone (LH),

Monocyte Chemotactic Protein 1 (MCP-1, CCL2),

Monocyte Chemotactic Protein 4 (MCP-4, CCL13),

Macrophage Derived Chemokine (MDC, CCL22),

Monokine Induced by Gamma Interferon (Mig, CXCL9),

Macrophage Inflammatory Protein 1 alpha (MIP-1α, CCL3),

Matrix Metalloproteinase 3 (MMP-3),

Myoglobin (Mb),

N terminal prohormone of brain natriuretic peptide (NT-proBNP),

Osteoprotegerin (OPG, TNFRSF11B),

Pulmonary and Activation Regulated Chemokine (PARC),

Progesterone (P4),

Prostasin (PRSS8),

Phosphoserine Aminotransferase (PSAT),

Stem Cell Factor (SCF),

Transferrin (TRF),

Transthyretin (TT)/Prealbumin (PREA),

Thymus Expressed Chemokine (TECK),

Trefoil Factor 3 (TFF3),

Tumor Necrosis Factor alpha (TNFα, TNF),

Tumor Necrosis Factor Receptor 1 (TNFR1),

Tumor Necrosis Factor Receptor 2 (TNFR2),

Tissue type Plasminogen activator (tPA, PLAT),

Urokinase type Plasminogen Activator (uPA),

Urokinase type Plasminogen Activator Receptor (uPAR, CD87),

Vascular Endothelial Growth Factor (VEGF),

von Willebrand Factor (VWF),

YKL-40 (CHI3L1) or polynucleotides encoding such polypeptides.

In some embodiments, the following panels are used for characterizing endometriosis: Transthyretin (TT)/prealbumin (PREA), Apolipoprotein A-1 (ApoA1), β2-Microglobulin (B2M), Transferrin (TRF), Cancer Antigen 125 (CA125), Human Epididymis Protein 4 (HE4), and/or Follicle Stimulating Hormone (FSH); and β2-microglobulin (B2M), Cancer Antigen 125 (CA125), Follicle Stimulating Hormone (FSH), Chemokine 4 (CCL4, MIP-1β), Immunoglobulin M (IgM), Luteinizing Hormone (LH), Macrophage Derived Chemokine (MDC, CCL22), and Progesterone (P4).

In some embodiments, the following panels are used for characterizing endometriosis: Cancer Antigen 125 (CA125), Apolipoprotein A-1 (ApoA1), Transferrin (TRF), β2-microglobulin (B2M), Follicle Stimulating Hormone (FSH), Human Epididymis Protein 4 (HE4), and Prealbumin (PREA). In some embodiments, the following panels are used for characterizing endometriosis: Cancer Antigen 125 (CA125), Macrophage Derived Chemokine (MDC, CCL22), Progesterone (P4), EN-RAGE (S100A12), Immunoglobulin M (IgM), Chemokine 4 (CCL4, MIP-1β, HCC4), β2-microglobulin (B2M), Follicle Stimulating Hormone (FSH), Luteinizing Hormone (LH), and Cystatin C (CST3). In some embodiments, the following panels are used for characterizing endometriosis: Cancer Antigen 125 (CA125), Macrophage Derived Chemokine (MDC, CCL22), Progesterone (P4), and Apolipoprotein A-1 (ApoA1). In some embodiments, the following panels are used for characterizing endometriosis: Cancer Antigen 125 (CA125), Apolipoprotein A1 (ApoA1), Macrophage Derived Chemokine (MDC, CCL22), Progesterone (P4), EN-RAGE (S100A12), Immunoglobulin M (IgM), Chemokine 4 (CCL4, MIP-1β, HCC4), β2-microglobulin (B2M), Follicle Stimulating Hormone (FSH), and Luteinizing Hormone (LH). In some embodiments, any of the panels used for characterizing endometriosis include age.

The invention further features the use of such panels for characterizing endometriosis. In particular, the use of such panels provides methods for non-invasively characterizing endometriosis in a subject.

### Endometriosis

Endometriosis is a debilitating estrogen-dependent gynecological disorder. The degree of endometriosis is staged according to the classification system of the American Society of Reproductive Medicine into, mild, moderate, and severe disease. The cause is not entirely clear, although the prevailing theory is Sampson's theory of retrograde menstruation.

There is no cure for endometriosis, but moderate to severe pain can be managed using, for example, elagolix, an oral gonadotropin-releasing hormone (GnRH) antagonist. Other treatments include GnRH antagonists, such as abarelix, cetrorelix, degarelix, ganirelix, and relugolix, as well as GnRH agonists, such as buserelin, gonadorelin, goserelin, histrelin, leuprorelin, nafarelin, and triptorelin.

The invention provides compositions and methods for the selection of subjects for treatment with GnRH agonists and antagonists.

### Biomarkers

In particular embodiments, a biomarker is an organic biomolecule that is differentially present in a sample taken from a subject of one phenotypic status (e.g., having a disease, such as endometriosis) as compared with another phenotypic status (e.g., not having the disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. Therefore, they are useful as markers for characterizing a disease (e.g., endometriosis).

### Biomarkers for Endometriosis

The invention provides panels of polypeptide biomarkers that are differentially present in subjects having endometriosis, and methods of using such panels to characterize a biological sample from a subject. The biomarkers of this invention are differentially present depending on endometriosis status, including, subjects having endometriosis vs. subjects that do not have endometriosis.

The biomarker panel of the invention comprises one or more of the biomarkers presented in the following Table 1.

TABLE 1

| Biomarker | Diff. Reg. in Endo |
|---|---|
| Alpha 1 Microglobulin (A1M) | Decreased |
| Alpha Fetoprotein (AFP) | Decreased |
| Angiopoietin 2 (Ang-2) | Decreased |
| Apolipoprotein B (ApoB) | Decreased |
| Apolipoprotein E (ApoE) | Decreased |
| β2-Microglobulin (B2M) | Decreased |
| Cystatin C (CST3) | Decreased |
| Cancer Antigen 125 (CA125, MUC16) | Increased |
| CA125 Glycoform | |
| CD 40 Antigen (CD40) | Decreased |
| Chromogranin A (CgA) | Decreased |
| Chemokine 4 (CCL4, MIP-1β) | Decreased |
| Clusterin (CLU, ApoJ) | Decreased |
| Eotaxin 1 (CCL11) | Decreased |
| Endostatin | Decreased |
| EN-RAGE (S100A12) | Increased |
| Fatty Acid Binding Protein | Decreased |
| (adipocyte)(FABP4) | |
| Fatty Acid Binding Protein | Decreased |
| (heart) (H-FABP, FABP3) | |
| Fas Ligand Receptor (Fas, | Decreased |
| FasR) | |
| Ferritin (FTL) | Decreased |
| Follicle Stimulating | Decreased |
| Hormone (FSH) | |
| Galectin 3 (Gal-3) | Decreased |
| Growth Hormone (GH, | Increased |
| somatotropin, human growth | |
| hormone, HGH) | |
| Glutathione S Transferase | Decreased |
| alpha (GSTα) | |
| Human Chorionic | Decreased |
| Gonadotropin (hCG) | |
| Hepatocyte Growth Factor | Decreased |
| (HGF) | |
| Haptoglobin (Hp) | Decreased |
| Immunoglobulin E (IgE) | Decreased |
| Immunoglobulin M (IgM) | Increased |
| Insulin like Growth Factor | Decreased |
| Binding Protein 4 (IGFBP4) | |
| Insulin like Growth Factor I | Increased |
| (IGF-I) | |
| Interleukin 8 (IL-8, CXCL8) | Decreased |
| Interferon gamma Induced | Decreased |
| Protein 10 (IP-10, CXCL10) | |
| Interferon inducible T cell | Decreased |
| alpha chemoattractant | |
| (I-TAC, CXCL11) | |
| Kallikrein 5 (KLK5) | Increased |
| Leptin (LEP) | Decreased |
| Luteinizing Hormone (LH) | Decreased |
| Monocyte Chemotactic | Decreased |
| Protein 1 (MCP-1, CCL2) | |
| Monocyte Chemotactic | Decreased |
| Protein 4 (MCP-4, CCL13) | |
| Macrophage Derived | Decreased |
| Chemokine (MDC, CCL22) | |
| Monokine Induced by | Decreased |
| Gamma Interferon (Mig, | |
| CXCL9) | |
| Macrophage Inflammatory | Decreased |
| Protein 1 alpha (MIP-1α, | |
| CCL3) | |
| Matrix Metalloproteinase 3 | Decreased |
| (MMP-3) | |
| Myoglobin (Mb) | Decreased |
| N terminal prohormone of | Decreased |
| brain natriuretic peptide | |
| (NT-proBNP) | |
| Osteoprotegerin (OPG, | Decreased |
| TNFRSF11B) | |
| Pulmonary and Activation | Decreased |
| Regulated Chemokine (PARC) | |
| Progesterone (P4) | Increased |
| Prostasin (PRSS8) | Decreased |
| Phosphoserine | Decreased |
| Aminotransferase (PSAT) | |
| Stem Cell Factor (SCF) | Decreased |
| Thymus Expressed | Decreased |
| Chemokine (TECK) | |
| Trefoil Factor 3 (TFF3) | Decreased |
| Tumor Necrosis Factor alpha | Decreased |
| (TNFα, TNF) | |
| Tumor Necrosis Factor | Decreased |
| Receptor 1 (TNFR1) | |
| Tumor Necrosis Factor | Decreased |
| Receptor 2 (TNFR2) | |
| Tissue type Plasminogen | Decreased |
| activator (tPA, PLAT) | |
| Urokinase type Plasminogen | Increased |
| Activator (uPA) | |
| Urokinase type Plasminogen | Decreased |
| Activator Receptor (uPAR, | |
| CD87) | |
| Vascular Endothelial Growth | Decreased |
| Factor (VEGF) | |
| von Willebrand Factor | Decreased |
| (VWF) | |
| YKL-40 (CHI3L1) | Decreased |
| Apolipoprotein A1 (ApoA1) | Altered/slightly decreased |
| Transferrin (TRF) | Altered |
| Transthyritin | Altered |
| (TT)/Prealbumin (PREA) | |
| Human Epididymis Protein 4 | Altered |
| (HE4, WFDC2) | |

As would be understood, references herein to a biomarker of Table 1, a panel of biomarkers, or other similar phrase indicates one or more of the biomarkers set forth in Table 1 or otherwise described herein.

A biomarker of the invention may be detected in a biological sample of the subject (e.g., tissue, fluid), including, but not limited to, blood, blood serum, plasma, saliva, urine, ascites, cyst fluid, a homogenized tissue sample (e.g., a tissue sample obtained by biopsy), a cell isolated from a patient sample, and the like.

The invention provides panels comprising isolated biomarkers. The biomarkers can be isolated from biological fluids, such as urine or serum. They can be isolated by any method known in the art. In certain embodiments, this isolation is accomplished using the mass and/or binding characteristics of the markers. For example, a sample comprising the biomolecules can be subject to chromatographic fractionation and subject to further separation by, e.g., acrylamide gel electrophoresis. Knowledge of the identity of the biomarker also allows their isolation by immunoaffinity chromatography. By “isolated biomarker” is meant at least 60%, by weight, free from proteins and naturally-occurring organic molecules with which the marker is naturally associated. Preferably, the preparation is at least 75%, more preferably 80, 85, 90 or 95% pure or at least 99%, by weight, a purified marker.

### Alpha 1 Microglobulin (A1M)

One exemplary biomarker present in the panel of the invention is A1M. A1M is a heme- and radical-binding protein found in all vertebrates, including man, which operates by rapidly clearing cytosols and extravascular fluids of heme groups and free radicals released from hemoglobin. A1M inhibits immunological functions of white blood cells in vitro, and its distribution is consistent with an anti-inflammatory and protective role in vivo. A1M is a 183 amino acid protein (UniProt Accession No. P02760), which is a cleavage product of the alpha-1-microglobulin/bikunin precursor (AMBP). In aspects of the invention, A1M is decreased in subjects with endometriosis compared to those with other benign conditions.

### Alpha Fetoprotein (AFP)

One exemplary biomarker present in the panel of the invention is AFP. AFP is one of several oncofetal proteins synthesized in large amounts by the fetus. Although synthesis drops markedly shortly after birth, small amounts of AFP continue to be produced in the adult. The function of AFP is unknown, but recent studies suggest the possibility that it may have immunoregulatory properties and/or may influence cell proliferation and growth. The high affinity of AFP for estrogen could have important biological functions, although the significance of this binding has not yet been clearly defined. Increased levels of AFP are seen in a variety of clinical situations, including pregnancy, hepatic disorders, especially chronic hepatitis, and various malignancies, particularly hepatomas, teratomas, and those of primitive gut origin. AFP is a 591 amino acid protein (UniProt Accession No. P02771). In aspects of the invention, AFP is decreased in subjects with endometriosis compared to those with other benign conditions.

### Angiopoietin 2 (Ang-2)

One exemplary biomarker present in the panel of the invention is Ang-2. Ang-2 disrupts the connections between the endothelium and perivascular cells and promotes cell death and vascular regression. Yet, in conjunction with VEGF, Ang-2 promotes neo-vascularization. Hence, angiopoietins exert crucial roles in the angiogenic switch during tumor progression. Ang-2 is a 496 amino acid protein (UniProt Accession No. P015123). In aspects of the invention, Ang-2 is decreased in subjects with endometriosis compared to those with other benign conditions.

### Apolipoprotein B (ApoB)

One exemplary biomarker present in the panel of the invention is ApoB. Plasma lipoprotein metabolism is regulated and controlled by the major apolipoproteins including apoE, apoB, apoA-I, apoA-II, apoA-IV, apoC-I, apoC-II, and apoC-III. Specific apolipoproteins function in the regulation of lipoprotein metabolism. ApoB is a 4563 amino acid protein (UniProt Accession No. P04114). In aspects of the invention, ApoB is decreased in subjects with endometriosis compared to those with other benign conditions.

### Apolipoprotein E (ApoE)

One exemplary biomarker present in the panel of the invention is ApoE. ApoE, a multifunctional protein with central roles in lipid metabolism and neurobiology, has three common isoforms (apoE2, apoE3, and apoE4) with different effects on lipid homeostasis and neurobiology. Unlike apoE3, the most common isoform, apoE4, is associated with increased risk of developing Alzheimer disease (AD) and other neurodegenerative disorders. ApoB is a 299 amino acid protein (UniProt Accession No. P02649). In aspects of the invention, ApoE is decreased in subjects with endometriosis compared to those with other benign conditions.

### β2-Microglobulin (B2M)

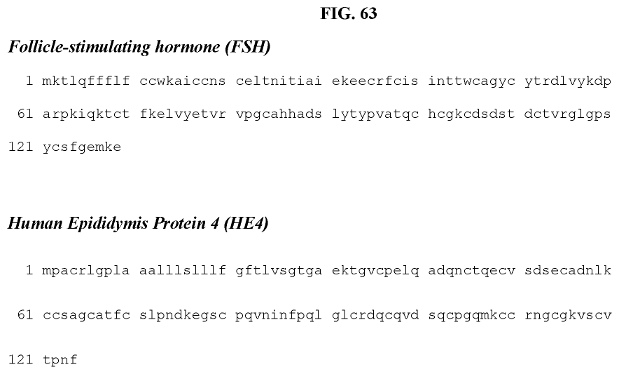

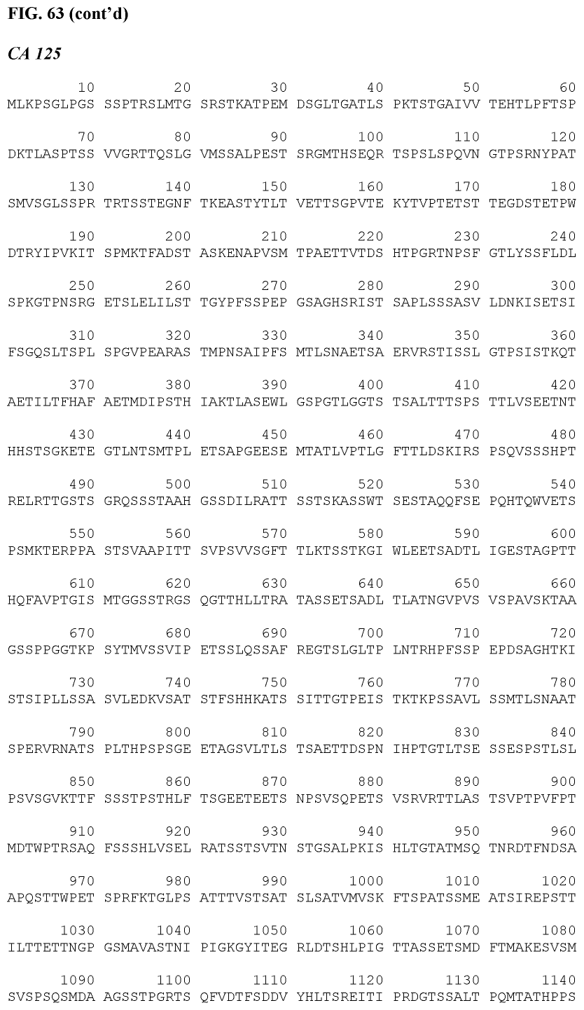

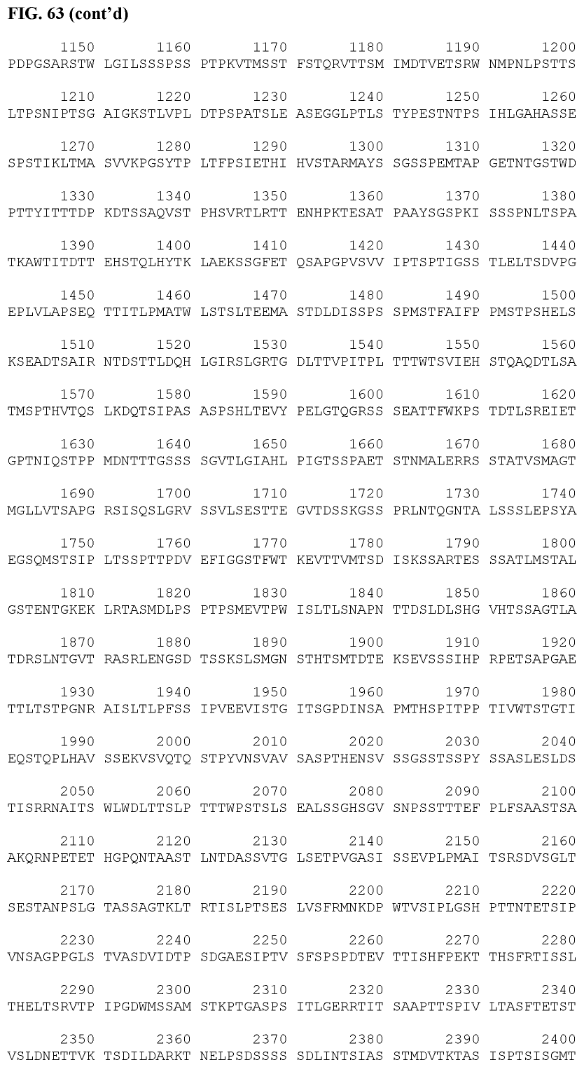

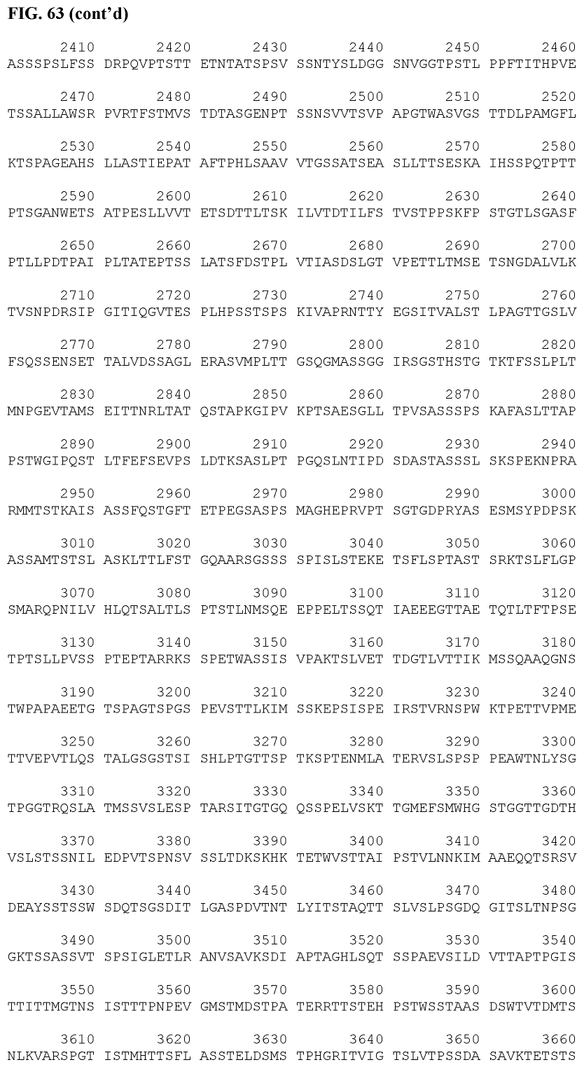

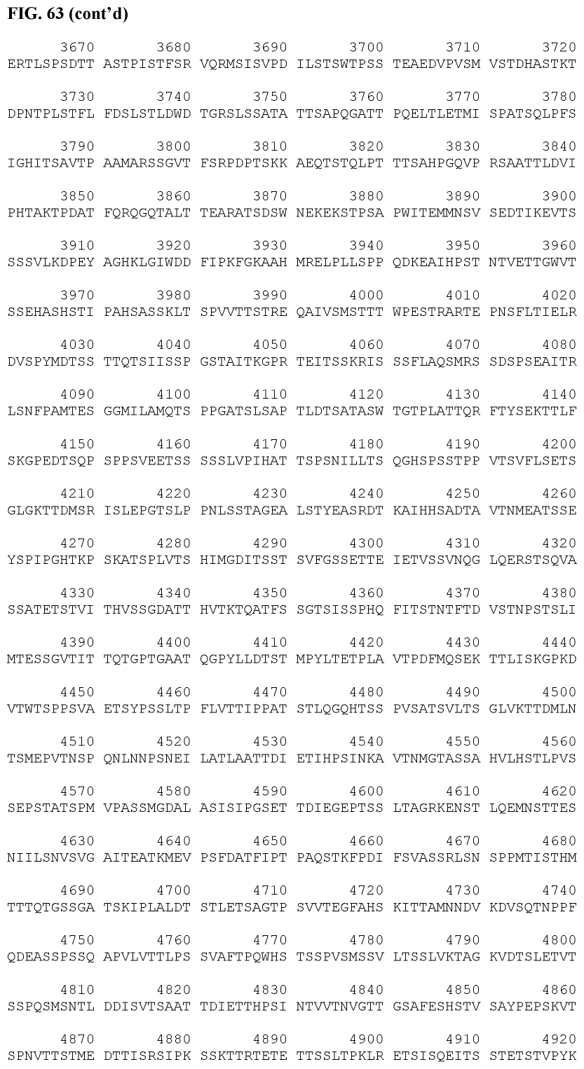

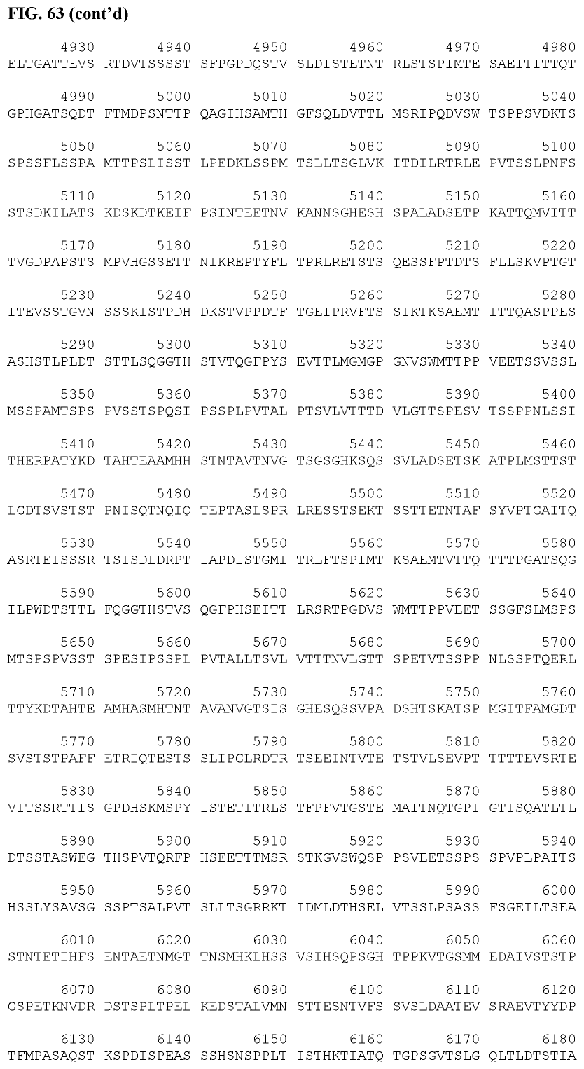

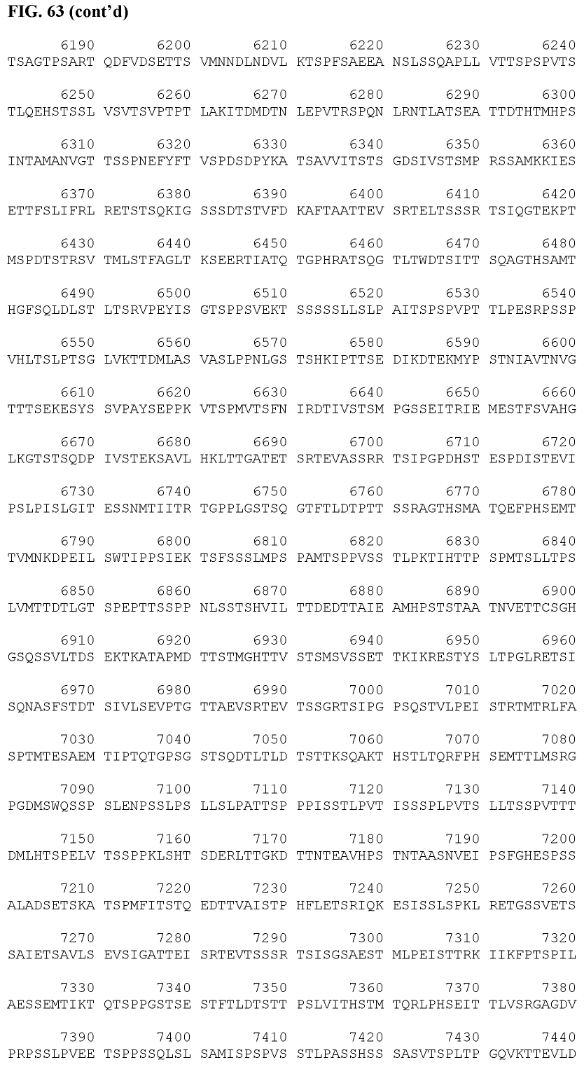

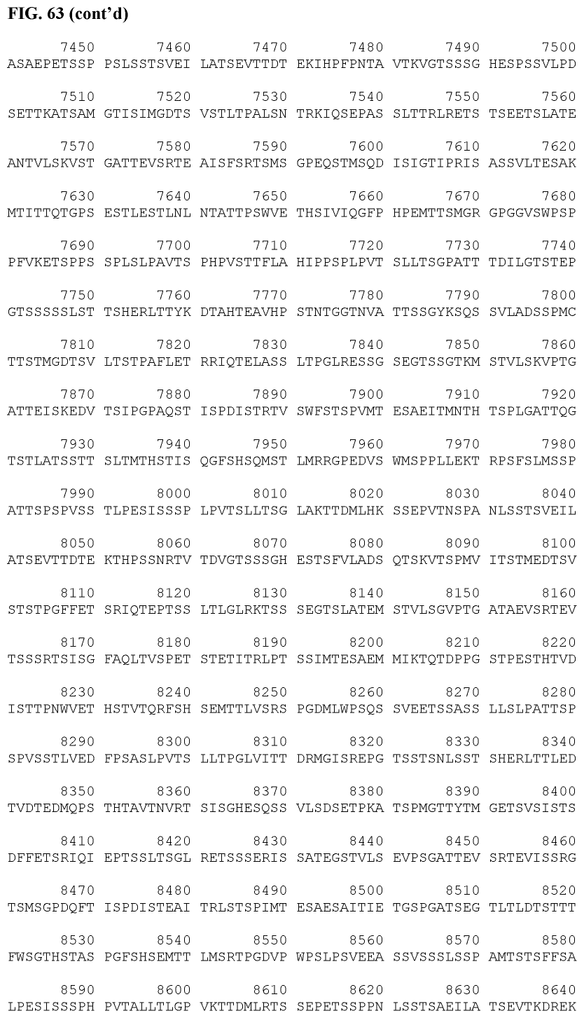

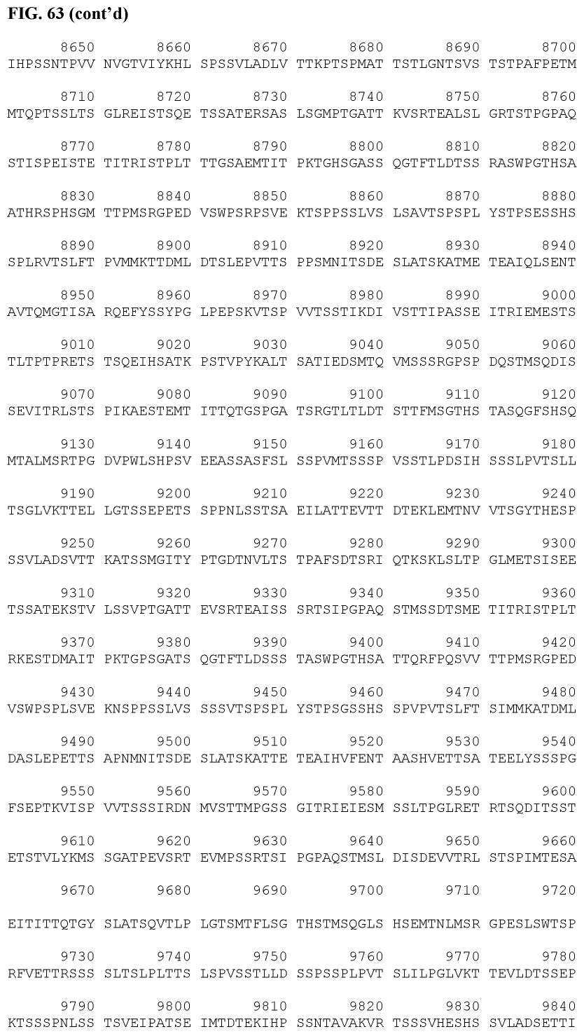

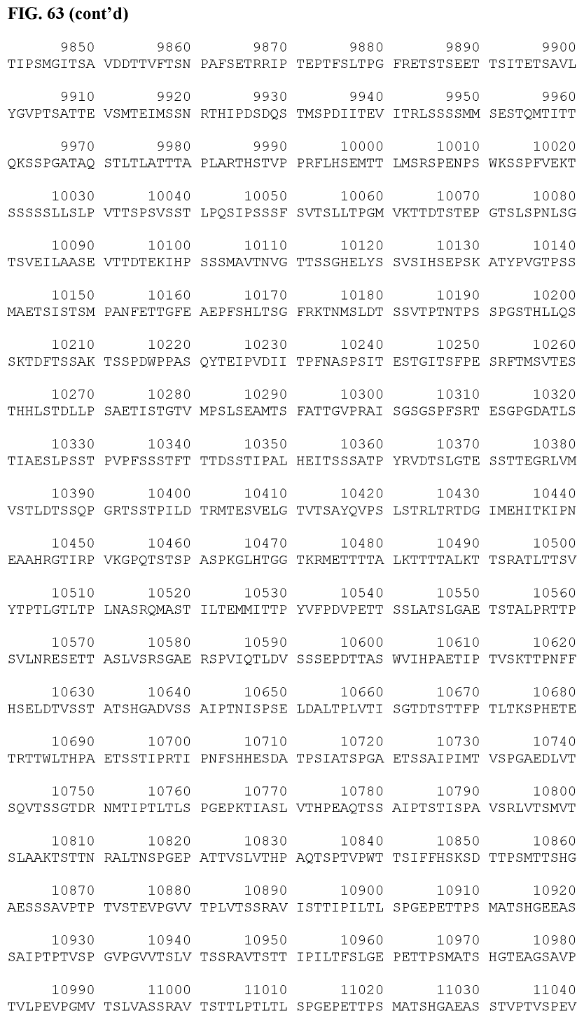

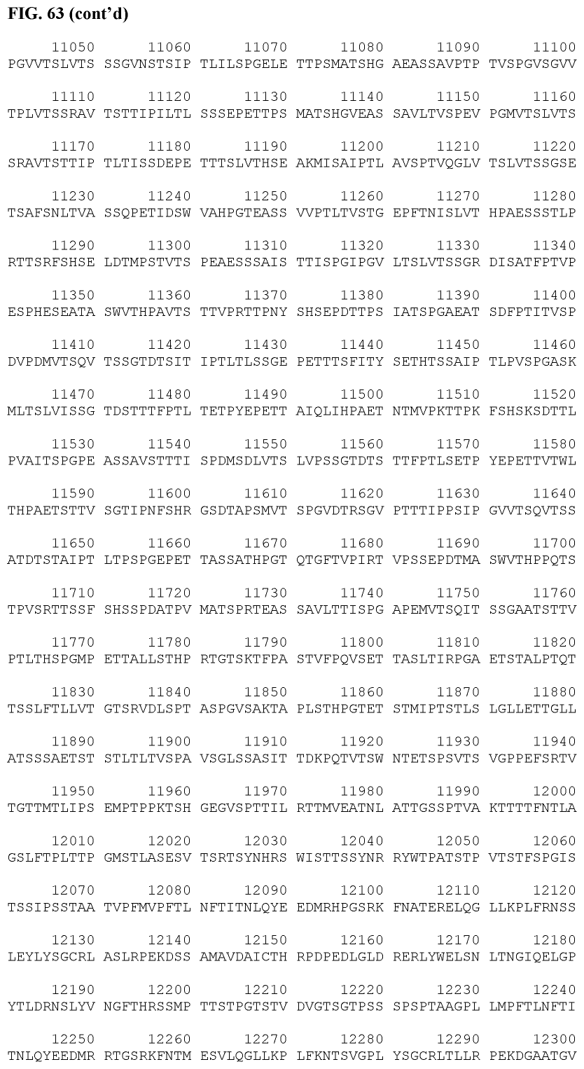

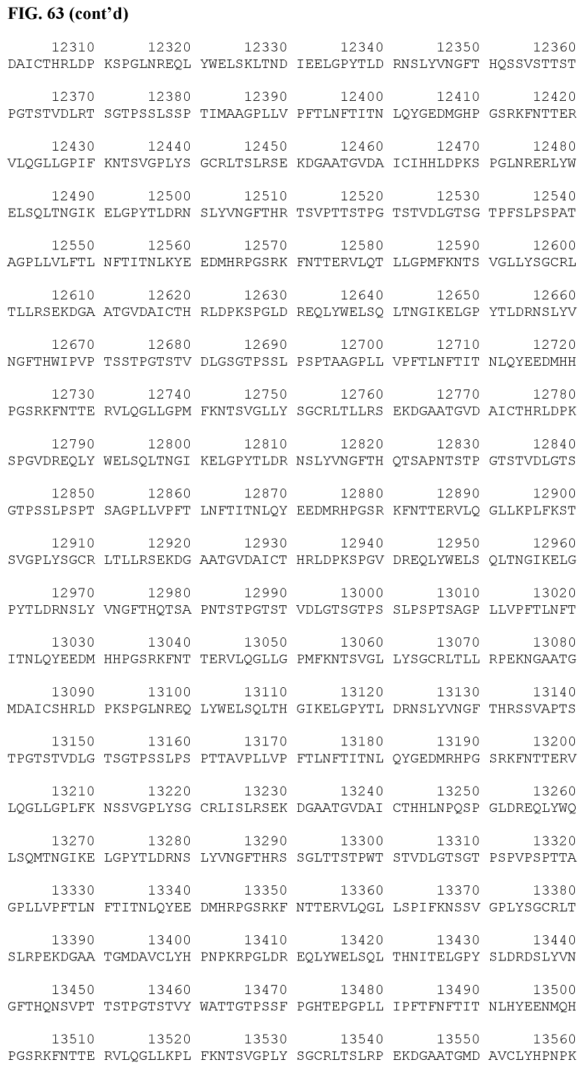

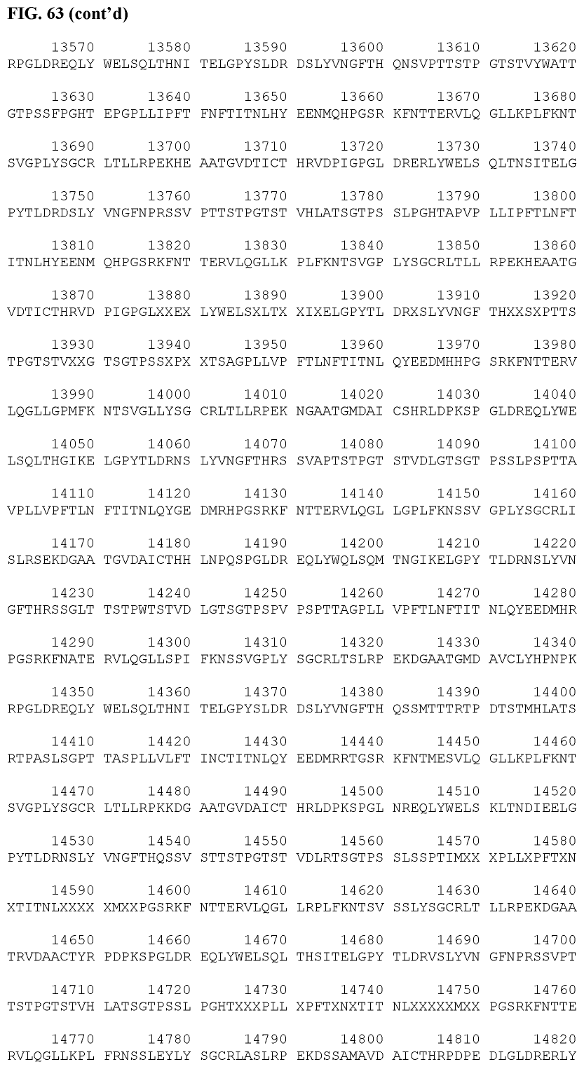

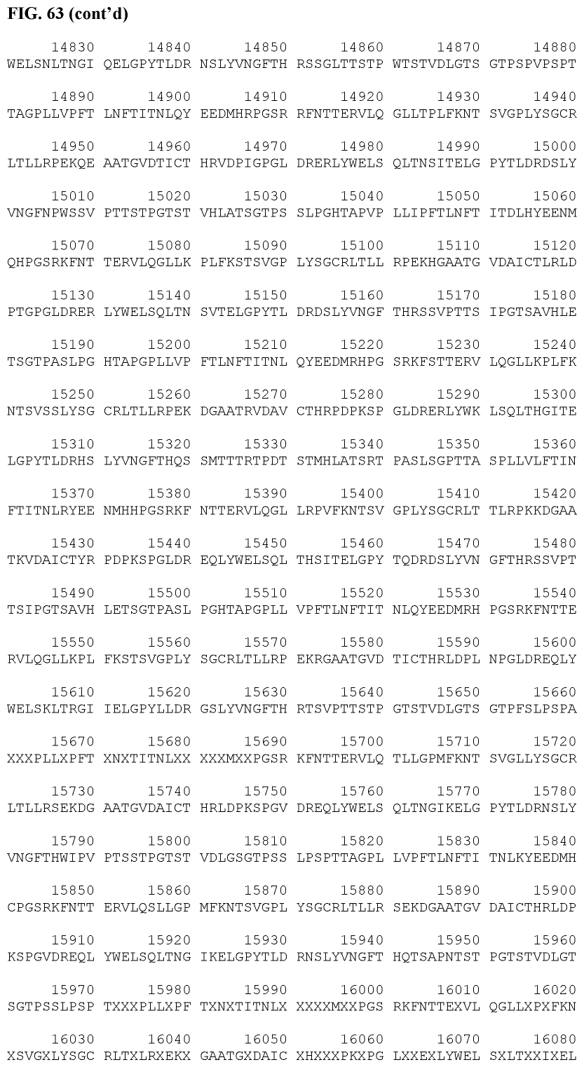

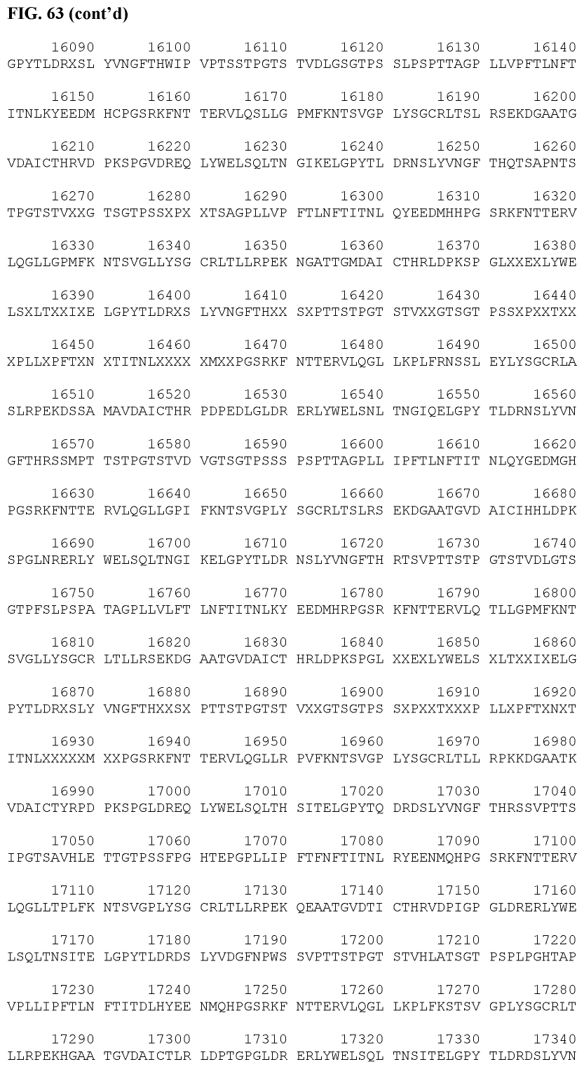

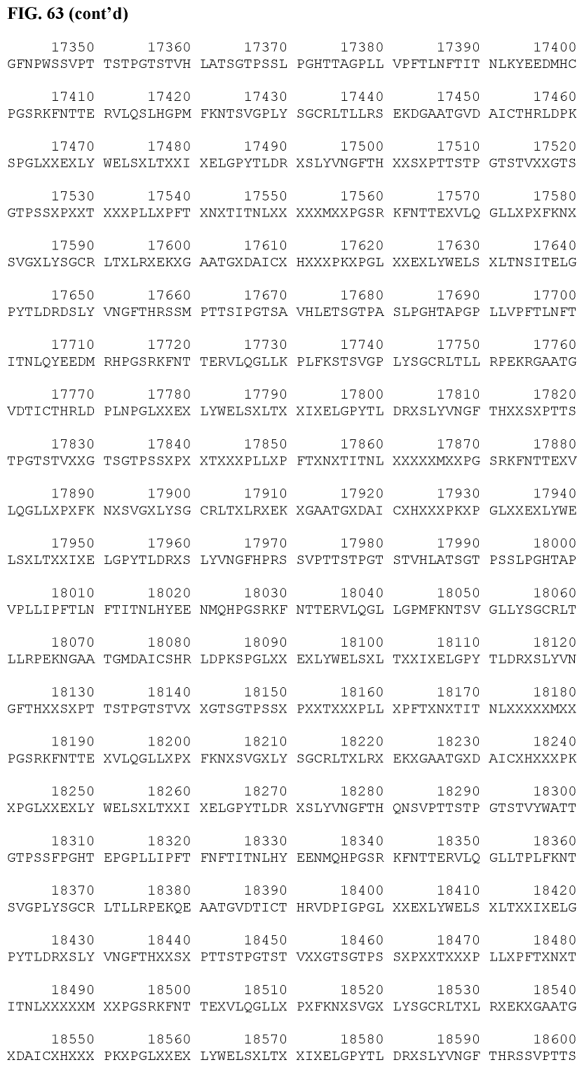

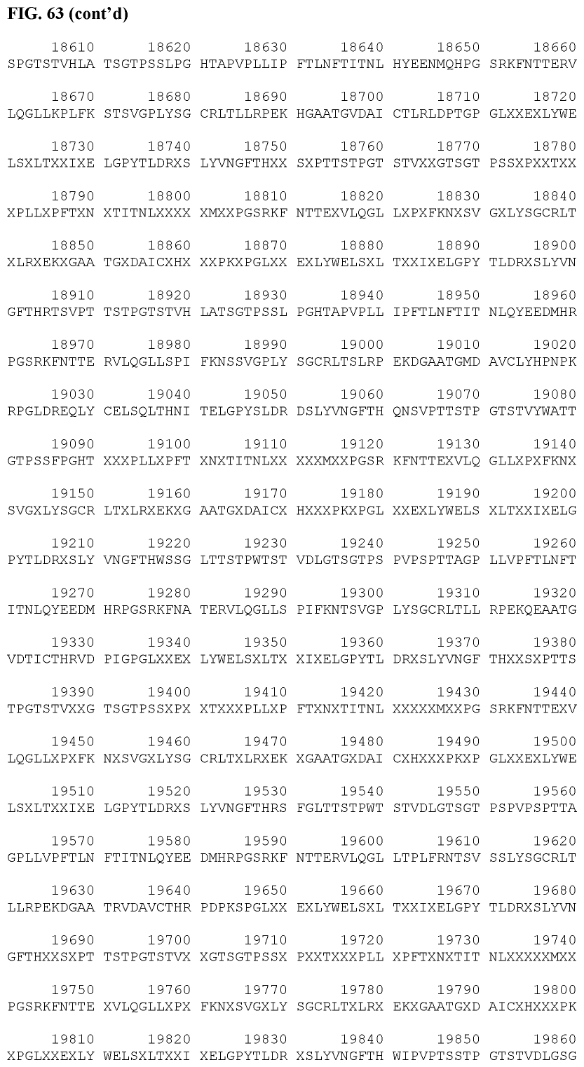

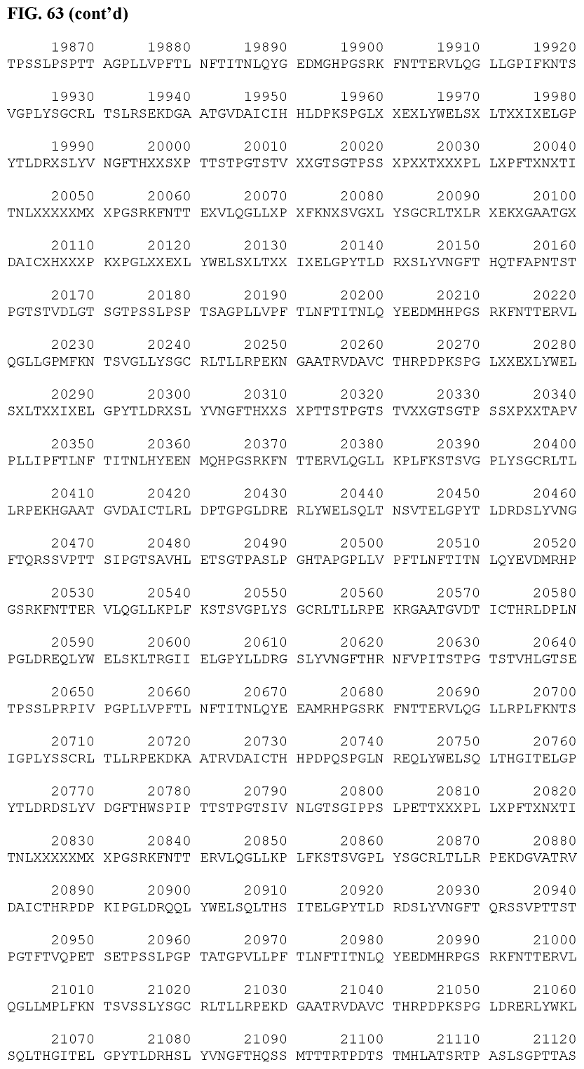

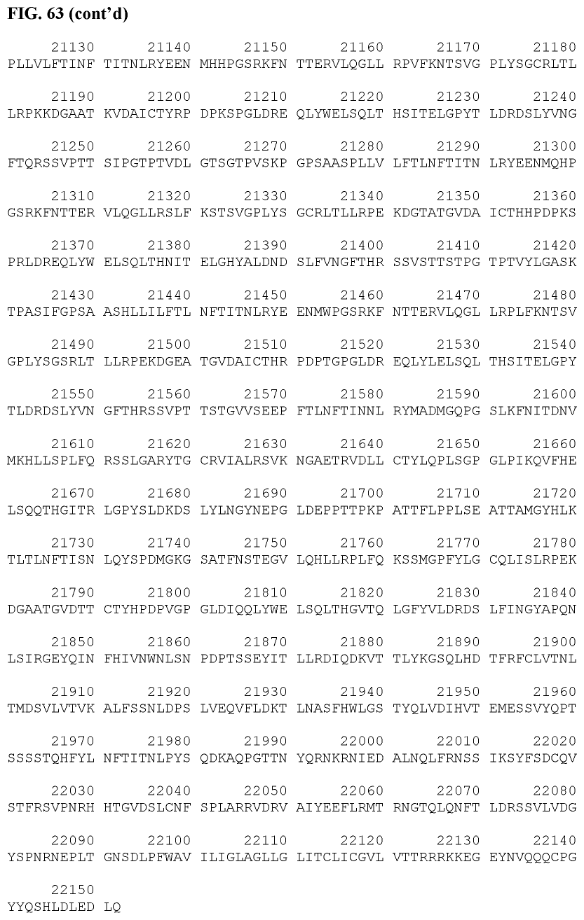

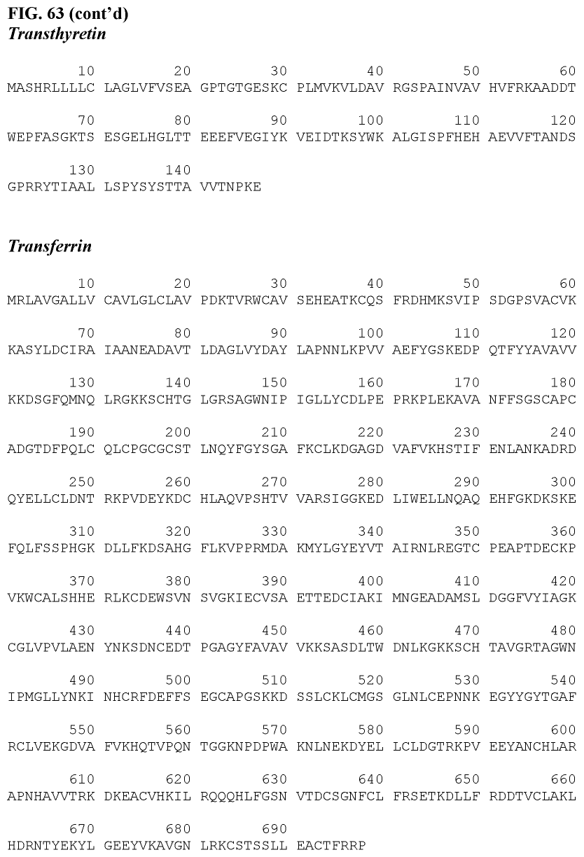

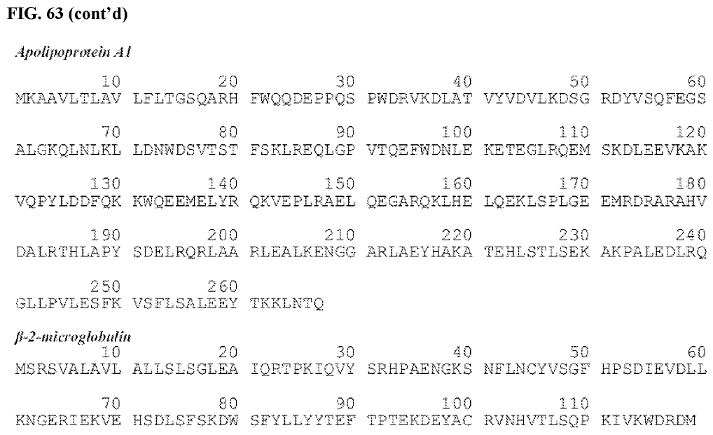

One exemplary biomarker present in the panel of the invention is B2M. B2M is a low molecular weight protein with sequence homology to immunoglobulins. As a portion of the HLA complex, this protein is an important cell-surface structure. Under normal conditions, B2M is synthesized and shed by many cells, particularly lymphocytes, and is detectable in the circulation of normal individuals. B2M is a 99 amino acid protein (UniProt Accession No. P61769). The amino acid sequence of an exemplary B2M polypeptide is set forth in FIG. 63. In aspects of the invention, B2M is decreased in subjects with endometriosis compared to those with other benign conditions. β2-microglobulin is recognized by antibodies. Such antibodies can be made using any method well known in the art, and can also be commercially purchased from, e.g., Abcam (catalog AB759) (www.abcam.com, Cambridge, Mass.).

### Cystatin C (CST3)

One exemplary biomarker present in the panel of the invention is cystatin C. The cystatin superfamily encompasses proteins that contain multiple cystatin-like sequences. Some of the members are active cysteine protease inhibitors, while others have lost or perhaps never acquired this inhibitory activity. There are three inhibitory families in the superfamily, including the type 1 cystatins (stefins), type 2 cystatins, and the kininogens. The type 2 cystatin proteins are a class of cysteine proteinase inhibitors found in a variety of human fluids and secretions, where they appear to provide protective functions. Expression of this protein in vascular wall smooth muscle cells is severely reduced in both atherosclerotic and aneurysmal aortic lesions, establishing its role in vascular disease. In addition, this protein has been shown to have an antimicrobial function, inhibiting the replication of herpes simplex virus. Cystatin is a 120 amino acid protein (UniProt Accession No. P01034). In aspects of the invention, cystatin is decreased in subjects with endometriosis compared to those with other benign conditions.

### Cancer Antigen 125 (CA125, MUC16)

One exemplary biomarker present in the panel of the invention is CA125. CA125, also known as MUC16, is most commonly known as a biomarker for ovarian cancer, though other cancers as well as a number of benign conditions also cause serum levels to be increased. CA125 is a component of the ocular surface, respiratory tract, and epithelia of the female reproductive tract. CA125 is a 22152 amino acid protein (UniProt Accession No. Q8WX17). The amino acid sequence of an exemplary CA125 polypeptide is set forth in FIG. 63. In aspects of the invention, CA125 is increased in subjects with endometriosis compared to those with other benign conditions.

### CD 40 Antigen (CD40)

One exemplary biomarker present in the panel of the invention is CD40. Because of its essential role in immunity, one of the best characterized of the costimulatory molecules is the receptor CD40. This receptor, a member of the tumor necrosis factor receptor family, is expressed by B cells, professional antigen-presenting cells, as well as non-immune cells and tumors. CD40 is a 277 amino acid protein (UniProt Accession No. P24952). In aspects of the invention, CD40 is decreased in subjects with endometriosis compared to those with other benign conditions.

### Chromogranin A (CgA)

One exemplary biomarker present in the panel of the invention is CgA. CgA is the major member of the granin family of acidic secretory glycoproteins that are expressed in all endocrine and neuroendocrine cells. Granins have been proposed to play multiple roles in the secretory process. Several biologically active peptides encoded within the CgA molecule, such as vasostatin, beta-granin, chromostatin, pancreastatin, and parastatin act predominantly to inhibit hormone and neurotransmitter release in an autocrine or paracrine fashion. CgA is a 457 amino acid protein (UniProt Accession No. P10645). In aspects of the invention, CgA is decreased in subjects with endometriosis compared to those with other benign conditions.

### Chemokine 4 (CCL4, MIP-1β)

One exemplary biomarker present in the panel of the invention is CCL4. CCL4 was the most potent chemoattractant of a CD4+CD25+ T cell population, which is a characteristic phenotype of regulatory T cells. Depletion of either regulatory T cells or CCL4 resulted in a deregulated humoral response, which culminated in the production of autoantibodies. This suggested that the recruitment of regulatory T cells to B cells and APCs by CCL4 plays a central role in the normal initiation of T cell and humoral responses, and failure to do this leads to autoimmune activation. CCL4 is a 92 amino acid protein (UniProt Accession No. P13236). In aspects of the invention, CCL4 is decreased in subjects with endometriosis compared to those with other benign conditions.

### Clusterin (CLU, ApoJ)

One exemplary biomarker present in the panel of the invention is CLU. The CLU protein is a secreted chaperone that can under some stress conditions also be found in the cell cytosol. It has been suggested to be involved in several basic biological events such as cell death, tumor progression, and neurodegenerative disorders. CLU is a 449 amino acid protein (UniProt Accession No. P10909). In aspects of the invention, CLU is decreased in subjects with endometriosis compared to those with other benign conditions.

### Eotaxin 1 (CCL11)

One exemplary biomarker present in the panel of the invention is eotaxin. Eotaxin is an eosinophil-specific chemoattractant that has been recently identified in rodent models of asthma and host response against tumors. CCL11 is a 97 amino acid protein (UniProt Accession No. P51671). In aspects of the invention, CCL11 is decreased in subjects with endometriosis compared to those with other benign conditions.

### Endostatin

One exemplary biomarker present in the panel of the invention is endostatin. Collagen XVIII is a component of basement membranes (BMs) with the structural properties of both a collagen and a proteoglycan. Proteolytic cleavage within its C-terminal domain releases a fragment, endostatin, which has been reported to have anti-angiogenesis effects. Endostatin is a 184 amino acid protein, which is a proteolytic cleavage product of collagen XVIII (UniProt Accession No. P39060). In aspects of the invention, endostatin is decreased in subjects with endometriosis compared to those with other benign conditions.

### EN-RAGE (S100A12)

One exemplary biomarker present in the panel of the invention is EN-RAGE. EN-RAGE is a ligand for the receptor for advanced glycation end products (RAGE) and may be involved in the development of diabetic macro- and micro-angiopathy. EN-RAGE is a 92 amino acid protein (UniProt Accession No. P80511). In aspects of the invention, EN-RAGE is increased in subjects with endometriosis compared to those with other benign conditions.

### Fatty Acid Binding Protein (Adipocyte)(FABP4)

One exemplary biomarker present in the panel of the invention is FABP4. FABP4 encodes the fatty acid binding protein found in adipocytes. Fatty acid binding proteins are a family of small, highly conserved, cytoplasmic proteins that bind long-chain fatty acids and other hydrophobic ligands. It is thought that FABPs roles include fatty acid uptake, transport, and metabolism. FABP4 is a 132 amino acid protein (UniProt Accession No. P15090). In aspects of the invention, FABP4 is decreased in subjects with endometriosis compared to those with other benign conditions.

### Fatty Acid Binding Protein (Heart)(H-FABP, FABP3)

One exemplary biomarker present in the panel of the invention is H-FABP. H-FABP, also known as FABP3, has been isolated from a wide range of tissues, including heart, skeletal muscle, brain, renal cortex, lung, testis, aorta, adrenal gland, mammary gland, placenta, ovary and brown adipose tissue. Studies in H-FABP-deficient mice showed that the uptake of fatty acids was severely inhibited in the heart and skeletal muscle, whereas plasma concentrations of free fatty acids were increased. Cardiac and skeletal muscle metabolism is reported to switch from fatty-acid oxidation towards glucose oxidation when there is an inability to obtain sufficient amounts of fatty acids. Consequently, H-FABP-deficient mice were rapidly fatigued and exhausted by exercise, showing a reduced tolerance to physical activity. Localized cardiac hypertrophy was also observed in the older animals. FABP3 is a 133 amino acid protein (UniProt Accession No. P05413). In aspects of the invention, FABP3 is decreased in subjects with endometriosis compared to those with other benign conditions.

### Fas Ligand Receptor (Fas, FasR)

One exemplary biomarker present in the panel of the invention is Fas. The protein encoded by this gene is a member of the TNF-receptor superfamily. This receptor contains a death domain. It has been shown to play a central role in the physiological regulation of programmed cell death and has been implicated in the pathogenesis of various malignancies and diseases of the immune system. Fas is a 335 amino acid protein (UniProt Accession No. P25445). In aspects of the invention, Fas is decreased in subjects with endometriosis compared to those with other benign conditions.

### Ferritin (FTL)

One exemplary biomarker present in the panel of the invention is ferritin. Ferritin is the key to the control of the amount of iron available to the body. Ferritin is a protein that stores iron and releases it in a controlled fashion. Hence, the body has a “buffer” against iron deficiency (if the blood has too little iron, ferritin can release more) and, to a lesser extent, iron overload (if the blood and tissues of the body have too much iron, ferritin can help to store the excess iron). Ferritin is a 175 amino acid protein (FTL UniProt Accession No. P02792), or a 183 amino acid protein (FTH1 UniProt Accession No. P02794). In aspects of the invention, ferritin is decreased in subjects with endometriosis compared to those with other benign conditions.

### Follicle Stimulating Hormone (FSH)

One exemplary biomarker present in the panel of the invention is FSH. FSH stimulates the maturation of ovarian follicles. Administration of FSH to humans and animals induces “superovulation”, or development of more than the usual number of mature follicles and hence, an increased number of mature gametes. FSH is also critical for sperm production. It supports the function of Sertoli cells, which in turn support many aspects of sperm cell maturation. FSH is a heterodimer protein containing a 116 amino acid a subunit (CGA UniProt Accession No. P01215) and a 129 amino acid 3 subunit (FSHB UniProt Accession NO. P01225). The amino acid sequence of an exemplary FSH polypeptide is set forth in FIG. 63. Antibodies to FSH can be made using any method well known in the art, or can be purchased from, for example, Santa Cruz Biotechnology, Inc. (e.g., Catalog Number sc-57149) (www.scbt.com, Santa Cruz, Calif.). In aspects of the invention, FSH is decreased in subjects with endometriosis compared to those with other benign conditions.

### Galectin 3 (Gal-3)

One exemplary biomarker present in the panel of the invention is Gal-3. Gal-3 is widely spread among different types of cells and tissues, found intracellularly in nucleus and cytoplasm or secreted via non-classical pathway outside of cell, thus being found on the cell surface or in the extracellular space. Through specific interactions with a variety of intra- and extracellular proteins galectin-3 affects numerous biological processes and seems to be involved in different physiological and pathophysiological conditions, such as development, immune reactions, and neoplastic transformation and metastasis. Gal-3 is a 250 amino acid protein (UniProt Accession No. P17931). In aspects of the invention, Gal-3 is decreased in subjects with endometriosis compared to those with other benign conditions.

### Growth Hormone (GH, Somatotropin, Human Growth Hormone, HGH)

One exemplary biomarker present in the panel of the invention is GH. GH, also called somatotropin or human growth hormone, is a peptide hormone secreted by the anterior lobe of the pituitary gland. It stimulates the growth of essentially all tissues of the body, including bone. GH is synthesized and secreted by anterior pituitary cells called somatotrophs, which release between one and two milligrams of the hormone each day. GH is vital for normal physical growth in children; its levels rise progressively during childhood and peak during the growth spurt that occurs in puberty. GH is a 217 amino acid protein (GH1 UniProt Accession No. P01241). In aspects of the invention, GH is increased in subjects with endometriosis compared to those with other benign conditions.

### Glutathione S Transferase Alpha (GSTα)

One exemplary biomarker present in the panel of the invention is GSTα. The glutathione transferases (GSTs; also known as glutathione S-transferases) are major phase II detoxification enzymes found mainly in the cytosol. In addition to their role in catalyzing the conjugation of electrophilic substrates to glutathione (GSH), these enzymes also carry out a range of other functions. They have peroxidase and isomerase activities, they can inhibit the Jun N-terminal kinase (thus protecting cells against H₂O₂-induced cell death), and they are able to bind non-catalytically a wide range of endogenous and exogenous ligands. GSTα is a 222 amino acid protein (GSTA1 UniProt Accession No. P08263). In aspects of the invention, GSTα is decreased in subjects with endometriosis compared to those with other benign conditions.

Human Chorionic Gonadotropin (hCG)

One exemplary biomarker present in the panel of the invention is hCG. hCG is predominantly active in pregnancy and fetal development. Emerging evidence has revealed endogenous functions not previously ascribed to hCG, including participation in ovulation and fertilization, implantation, placentation and other activities in support of successful pregnancy. hCG is heterodimer protein containing a 116 amino acid α subunit (CGA UniProt Accession No. P01215) and a 165 amino acid 3 subunit (CBG3 UniProt Accession No. P01233). In aspects of the invention, hCG is decreased in subjects with endometriosis compared to those with other benign conditions.

### Hepatocyte Growth Factor (HGF)

One exemplary biomarker present in the panel of the invention is HGF. This gene encodes a protein that binds to the hepatocyte growth factor receptor to regulate cell growth, cell motility and morphogenesis in numerous cell and tissue types. Alternative splicing results in multiple transcript variants, at least one of which encodes a preproprotein that is proteolytically processed to generate alpha and beta chains, which form the mature heterodimer. This protein is secreted by mesenchymal cells and acts as a multi-functional cytokine on cells of mainly epithelial origin. This protein also plays a role in angiogenesis, tumorogenesis, and tissue regeneration. HGF is a 728 amino acid protein (UniProt Accession No. P14210). In aspects of the invention, HGF is decreased in subjects with endometriosis compared to those with other benign conditions.

### Haptoglobin (Hp)

One exemplary biomarker present in the panel of the invention is Hp. Hp is an acute phase protein capable of binding hemoglobin, thus preventing iron loss and renal damage.

Haptoglobin also acts as an antioxidant, has antibacterial activity and plays a role in modulating many aspects of the acute phase response. There are 3 major haptoglobin phenotypes: Hp(1-1), Hp(2-1) and Hp(2-2). Possession of a particular phenotype has been associated with a variety of common disorders (e.g., cardiovascular disease, autoimmune disorders, malignancy), a fact which can only be explained by the idea that possession of a particular phenotype offers some protection against the development of these disorders. Hp is a 406 amino acid protein (UniProt Accession No. P00738). In aspects of the invention, Hp is decreased in subjects with endometriosis compared to those with other benign conditions.

### Immunoglobulin E (IgE)

One exemplary biomarker present in the panel of the invention is IgE. IgE is a type of antibody found in mammals which is primarily involved in the immune response against parasites. It also has a role in hypersensitivity, making it a major player in many allergic responses. In aspects of the invention, IgE is decreased in subjects with endometriosis compared to those with other benign conditions.

### Insulin Like Growth Factor Binding Protein 4 (IGFBP4)

One exemplary biomarker present in the panel of the invention is IGFBP4. This gene is a member of the insulin-like growth factor binding protein (IGFBP) family and encodes a protein with an IGFBP domain and a thyroglobulin type-I domain. The protein binds both insulin-like growth factors (IGFs) I and II and circulates in the plasma in both glycosylated and non-glycosylated forms. Binding of this protein prolongs the half-life of the IGFs and alters their interaction with cell surface receptors. IGFBP4 is a 258 amino acid protein (UniProt Accession No. P22692). In aspects of the invention, IGFBP4 is decreased in subjects with endometriosis compared to those with other benign conditions.

### Insulin Like Growth Factor I (IGF-I)

One exemplary biomarker present in the panel of the invention is IGF-I. IGF-I and IGF-II, their binding proteins (IGFBPs), and the receptors mediating their signaling (types I and II IGF-IR), play critical roles in normal development, growth, metabolism, and homeostasis. The IGF-I pathway exerts such diverse influence on mammalian biology that the scope of its function is only now beginning to be understood. It has been insinuated in fundamental processes such as determining life span and coping with oxidative stress in rodents. IGF-I is a 195 amino acid protein (UniProt Accession No. P05019). In aspects of the invention, IGF-I is increased in subjects with endometriosis compared to those with other benign conditions.

### Immunoglobulin M (IgM)

One exemplary biomarker present in the panel of the invention is IgM. IgM is an antibody and part of the immune system produced by B cells. It is one of the earliest antibodies to be produced in an immune response. In aspects of the invention, IgM is increased in subjects with endometriosis compared to those with other benign conditions.

### Interleukin 8 (IL-8, CXCL8)

One exemplary biomarker present in the panel of the invention is IL-8. IL-8 is a chemoattractant cytokine produced by a variety of tissue and blood cells. Unlike many other cytokines, it has a distinct target specificity for the neutrophil, with only weak effects on other blood cells. IL-8 attracts and activates neutrophils in inflammatory regions. IL-8 is a 99 amino acid protein (UniProt Accession No. P10145). In aspects of the invention, IL-8 is decreased in subjects with endometriosis compared to those with other benign conditions.

### Interferon Gamma Induced Protein 10 (IP-10, CXCL10)

One exemplary biomarker present in the panel of the invention is IP-10, or CXCL10. Chemokines, which are a subfamily of the cytokines, act as chemoattractants for a wide variety of cells, including immune cells. CXCL10 is a small protein that is defined as an “inflammatory” chemokine and binds to CXCR3 to mediate immune responses through the activation and recruitment of leukocytes such as T cells, eosinophils, monocytes and NK cells. IP-10 is a 98 amino acid protein (UniProt Accession No. P02778). In aspects of the invention, IP-10 is decreased in subjects with endometriosis compared to those with other benign conditions.

### Interferon Inducible T Cell Alpha Chemoattractant (I-TAC, CXCL11)

One exemplary biomarker present in the panel of the invention is I-TAC. This novel chemokine is regulated by interferon (IFN) and has potent chemoattractant activity for IL-2-activated T cells. I-TAC could be a major chemoattractant for effector T cells involved in the pathophysiology of neuroinflammatory disorders, although I-TAC may also play a role in the migration of activated T cells during IFN-dominated immune responses. I-TAC is a 94 amino acid protein (UniProt Accession No. 014625). In aspects of the invention, I-TAC is decreased in subjects with endometriosis compared to those with other benign conditions.

### Kallikrein 5 (KLK5)

One exemplary biomarker present in the panel of the invention is KLK5. In normal human tissues, KLK5 is highly expressed in skin, mammary gland and testis. KLK5 is a 293 amino acid protein (UniProt Accession No. Q9Y337). In aspects of the invention, KLK5 is increased in subjects with endometriosis compared to those with other benign conditions.

### Leptin (LEP)

One exemplary biomarker present in the panel of the invention is leptin. Leptin is an adipocyte-derived hormone that acts as a major regulator for food intake and energy homeostasis. Leptin deficiency or resistance can result in profound obesity, diabetes, and infertility in humans. Since its discovery, our understanding of leptin's biological functions has expanded from anti-obesity to broad effects on reproduction, hematopoiesis, angiogenesis, blood pressure, bone mass, lymphoid organ homeostasis, and T lymphocyte systems. LEP is a 167 amino acid protein (UniProt Accession No. P41159). In aspects of the invention, LEP is decreased in subjects with endometriosis compared to those with other benign conditions

### Luteinizing Hormone (LH)

One exemplary biomarker present in the panel of the invention is LH. In both sexes, LH stimulates secretion of sex steroids from the gonads. In the testes, LH binds to receptors on Leydig cells, stimulating synthesis and secretion of testosterone. Theca cells in the ovary respond to LH stimulation by secretion of testosterone, which is converted into estrogen by adjacent granulosa cells. The name luteinizing hormone derives from this effect of inducing luteinization of ovarian follicles. LH is a heterodimer protein containing a 116 amino acid a subunit (CGA UniProt Accession No. P01215) and a 141 amino acid 3 subunit (LHB UniProt. Accession No. P01229). In aspects of the invention, LH is decreased in subjects with endometriosis compared to those with other benign conditions.

### Monocyte Chemotactic Protein 1 (MCP-1, CCL2)

One exemplary biomarker present in the panel of the invention is MCP-1. MCP-1, also known as CCL2), is one of the key chemokines that regulate migration and infiltration of monocytes/macrophages. Both CCL2 and its receptor CCR2 have been demonstrated to be induced and involved in various diseases. Migration of monocytes from the blood stream across the vascular endothelium is required for routine immunological surveillance of tissues, as well as in response to inflammation. MCP-1 is a 99 amino acid protein (UniProt Accession No. P13500). In aspects of the invention, MCP-1 is decreased in subjects with endometriosis compared to those with other benign conditions.

### Monocyte Chemotactic Protein 4 (MCP-4, CCL13)

One exemplary biomarker present in the panel of the invention is MCP-4. MCP-4 is an important chemoattractant for monocytes and T cells. Recent data indicate a role in renal inflammation. Expression of MCP-4 was up-regulated in response to the pro-inflammatory cytokines, TNF-alpha, and IFN-gamma. MCP-4 is a 98 amino acid protein (UniProt Accession No. Q99616). In aspects of the invention, MCP-4 is decreased in subjects with endometriosis compared to those with other benign conditions.

### Macrophage Derived Chemokine (MDC, CCL22)

One exemplary biomarker present in the panel of the invention is MDC. MDC is a CC chemokine paradigmatic of emerging aspects of chemokine immunobiology. It is constitutively expressed, yet microbial products and cytokines regulate its expression with divergent effects of type II (IL-4 and IL-13) and type I (interferon) cytokines. Processing of the mature protein by dipeptidyl peptidase IV/CD26 provides a further level of regulation. It acts on diverse cellular targets including dendritic cells (DC), NK cells, and T cell subsets. Among these, MDC is a potent attractant for CCR4 expressing polarized Th2 and Tc2 cells, and evidence is consistent with a role of this chemokine as an amplification loop of polarized type II responses. MDC is a 93 amino acid protein (UniProt Accession No. 000626). In aspects of the invention, MDC is decreased in subjects with endometriosis compared to those with other benign conditions.

### Monokine Induced by Gamma Interferon (Mig, CXCL9)

One exemplary biomarker present in the panel of the invention is Mig. Mig, the monokine induced by interferon-gamma, is a CXC chemokine active as a chemoattractant for activated T cells. Mig is related functionally to interferon-inducible protein 10 (IP-10), with which it shares a receptor, CXCR3. Previously, IP-10 was found to have antitumor activity in vivo. Mig is a 125 amino acid protein (UniProt Accession No. Q07325). In aspects of the invention, Mig is decreased in subjects with endometriosis compared to those with other benign conditions.

### Macrophage Inflammatory Protein 1 Alpha (MIP-1α, CCL3)

One exemplary biomarker present in the panel of the invention is MIP-1α. MIP-1α and MIP-1β are highly related members of the CC chemokine subfamily. Despite their structural similarities, MIP-1α and MIP-1β show diverging signaling capacities. Depending on the MIP-1 subtype and its NH₂-terminal processing, one or more of the CC chemokine receptors CCR1, CCR2, CCR3 and CCR5 are recognized. Since both human MIP-1α subtypes (LD78alpha and LD78beta) and MIP-1β signal through CCR5, the major co-receptor for M-tropic HIV-1 strains, these chemokines are capable of inhibiting HIV-1 infection in susceptible cells. MIP-1α is a 92 amino acid protein (UniProt Accession No. P10147). In aspects of the invention, MIP-1α is decreased in subjects with endometriosis compared to those with other benign conditions.

### Matrix Metalloproteinase 3 (MMP-3)

One exemplary biomarker present in the panel of the invention is MMP-3. The matrix metalloproteinases are a tightly regulated family of enzymes that degrade extracellular matrix and basement membrane components. Recent evidence suggests that these proteases and their specific inhibitors play important roles in normal developmental processes and in pathological conditions. Interestingly, experiments designed to improve our understanding of metalloproteinase regulation have also resulted in new insights into mechanisms by which growth factors and proto-oncogenes may regulate biological processes. MMP-3 is a 477 amino acid protein (UniProt Accession No. P08254). In aspects of the invention, MMP-3 is decreased in subjects with endometriosis compared to those with other benign conditions.

### Myoglobin (Mb)

One exemplary biomarker present in the panel of the invention is myoglobin. Myoglobin is a cytoplasmic hemoprotein, expressed solely in cardiac myocytes and oxidative skeletal muscle fibers, that reversibly binds O₂ by its heme residue, a porphyrin ring:iron ion complex. Since the initial discovery of its structure over 40 years ago, wide-ranging work by many investigators has added importantly to our understanding of its function and regulation. Functionally, myoglobin is well accepted as an O₂-storage protein in muscle, capable of releasing O₂ during periods of hypoxia or anoxia. Myoglobin is also thought to buffer intracellular O₂ concentration when muscle activity increases and to facilitate intracellular O₂ diffusion by providing a parallel path that augments simple diffusion of dissolved O₂. The use of gene targeting and other molecular biological techniques has revealed important new insights into the developmental and environmental regulation of myoglobin and provided additional functions for this hemoprotein such as scavenging nitric oxide and reactive O₂ species. Mb is a 154 amino acid protein (UniProt Accession No. P02144). In aspects of the invention, Mb is decreased in subjects with endometriosis compared to those with other benign conditions.

### N Terminal Prohormone of Brain Natriuretic Peptide (NT-proBNP)

One exemplary biomarker present in the panel of the invention is NT-proBNP. NT-proBNP testing is useful for diagnosing acute decompensated heart failure. NT-proBNP is a 134 amino acid protein (UniProt Accession No. P16860). In aspects of the invention, NT-proBNP is decreased in subjects with endometriosis compared to those with other benign conditions.

### Osteoprotegerin (OPG, TNFRSF11B)

One exemplary biomarker present in the panel of the invention is OPG. OPG is a member of the tumor necrosis factor receptor superfamily. OPG has an important function as a protector of bone, demonstrated by the fact that OPG(−/−) mice have severe osteoporosis.

OPG acts as a decoy receptor, binding to RANK ligand (RANKL), thus preventing the interaction between receptor activator of NF-kappaB (RANK) and RANKL. This interaction is required for the development of functionally active osteoclasts. OPG is a 401 amino acid protein (UniProt Accession No. 000300). In aspects of the invention, OPG is decreased in subjects with endometriosis compared to those with other benign conditions.

### Pulmonary and Activation Regulated Chemokine (PARC)

One exemplary biomarker present in the panel of the invention is pulmonary activation-regulated chemokine (PARC) now designated CC-chemokine ligand 18 (CCL18). CCL18 has been shown to play a significant role in the pathogenesis of various tissue injuries and diseases in a proinflammatory or immune suppressive way to limit or support the inflammation or disease. PARC is a 89 amino acid protein (UniProt Accession No. P55774). In aspects of the invention, PARC is decreased in subjects with endometriosis compared to those with other benign conditions.

### Progesterone (P4)

One exemplary biomarker present in the panel of the invention is progesterone (CAS Registry No. 57-83-0). Progesterone is a sex hormone involved in the regulation of the menstrual cycle and pregnancy. In aspects of the invention, progesterone is increased in subjects with endometriosis compared to those with other benign conditions.

### Prostasin (PRSS8)

One exemplary biomarker present in the panel of the invention is prostasin. Prostasin is a glycophosphatidylinositol-anchored protein which is found in prostate gland, kidney, bronchi, colon, liver, lung, pancreas, and salivary glands. It is a serine protease with trypsin-like substrate specificity which was first purified from seminal fluid in 1994. In the last decade, its diverse roles in various biological and physiological processes have been elucidated. Many studies done to date suggest that prostasin is one of several membrane peptidases regulating epithelial sodium channels in mammals. Prostasin is a 343 amino acid protein (UniProt Accession No. Q16651). In aspects of the invention, prostasin is decreased in subjects with endometriosis compared to those with other benign conditions.

### Phosphoserine Aminotransferase (PSAT)

One exemplary biomarker present in the panel of the invention is PSAT. PSAT is a catalyzing enzyme in the synthesis of serine. PSAT is a 370 amino acid protein (UniProt Accession No. Q9Y617). In aspects of the invention, PSAT is decreased in subjects with endometriosis compared to those with other benign conditions.

### Stem Cell Factor (SCF)

One exemplary biomarker present in the panel of the invention is SCF. SCF is an essential hematopoietic cytokine that interacts with other cytokines to preserve the viability of hematopoietic stem and progenitor cells, to influence their entry into the cell cycle and to facilitate their proliferation and differentiation. SCF on its own cannot drive noncycling hematopoietic progenitor cells into the cell cycle but does prevent their apoptotic death. SCF when combined with other cytokines increases the cloning efficacy of hematopoietic progenitor cells from all lineages. SCF also stimulates the growth of CD34+ leukemic progenitor cells from most patients with acute myeloid leukemia (AML). SCF is a 273 amino acid protein (UniProt Accession No. P21583). In aspects of the invention, SCF is decreased in subjects with endometriosis compared to those with other benign conditions.

### Thymus Expressed Chemokine (TECK)

One exemplary biomarker present in the panel of the invention is TECK. This antimicrobial gene belongs to the subfamily of small cytokine CC genes. Cytokines are a family of secreted proteins involved in immunoregulatory and inflammatory processes. The CC cytokines are proteins characterized by two adjacent cysteines. The cytokine encoded by this gene displays chemotactic activity for dendritic cells, thymocytes, and activated macrophages but is inactive on peripheral blood lymphocytes and neutrophils. The product of this gene binds to chemokine receptor CCR9. Alternative splicing results in multiple transcript variants. TECK is a 150 amino acid protein (UniProt Accession No. 015444). In aspects of the invention, TECK is decreased in subjects with endometriosis compared to those with other benign conditions.

### Trefoil Factor 3 (TFF3)

One exemplary biomarker present in the panel of the invention is TFF. Trefoil factors are secretory products of mucin producing cells. They play a key role in the maintenance of the surface integrity of oral mucosa and enhance healing of the gastrointestinal mucosa by a process called restitution. TFF comprises the gastric peptides (TFF1), spasmolytic peptide (TFF2), and the intestinal trefoil factor (TFF3). They have an important and necessary role in epithelial restitution within the gastrointestinal tract. Significant amounts of TFF are present in human milk. TFF3 is a 94 amino acid protein (UniProt Accession No. 007654). In aspects of the invention, TFF3 is decreased in subjects with endometriosis compared to those with other benign conditions.

### Tumor Necrosis Factor Alpha (TNFα, TNF)

One exemplary biomarker present in the panel of the invention is TNFα. TNFα is a multifunctional proinflammatory cytokine that belongs to the tumor necrosis factor (TNF) superfamily. This cytokine is mainly secreted by macrophages. It can bind to, and thus functions through its receptors TNFRSF1A/TNFR1 and TNFRSF1B/TNFBR. This cytokine is involved in the regulation of a wide spectrum of biological processes including cell proliferation, differentiation, apoptosis, lipid metabolism, and coagulation. TNFα is a 233 amino acid protein (UniProt Accession No. P01375). In aspects of the invention, TNFα is decreased in subjects with endometriosis compared to those with other benign conditions.

### Tumor Necrosis Factor Receptor 1 (TNFR1)

One exemplary biomarker present in the panel of the invention is TNFR1. TNFR1 is a binding protein which binds several proteins in the tumor necrosis factor family including TNFα. TNFR1 is a 455 amino acid protein (UniProt Accession No. P19438). In aspects of the invention, TNFR1 is decreased in subjects with endometriosis compared to those with other benign conditions.

### Tumor Necrosis Factor Receptor 2 (TNFR2)

One exemplary biomarker present in the panel of the invention is TNFR2. TNFR2 is a binding protein which binds several proteins in the tumor necrosis factor family including TNFα. TNFR2 is a 461 amino acid protein (UniProt Accession No. P20333). In aspects of the invention, TNFR2 is decreased in subjects with endometriosis compared to those with other benign conditions.

Tissue Type Plasminogen Activator (tPA, PLAT)

One exemplary biomarker present in the panel of the invention is tPA. tPA is a serine protease constituted of five functional domains through which it interacts with different substrates, binding proteins, and receptors. In the last years, great interest has been given to the clinical relevance of targeting tPA in different diseases of the central nervous system, in particular, stroke. Among its reported functions in the central nervous system, tPA displays both neurotrophic and neurotoxic effects. tPA is a 562 amino acid protein (UniProt Accession No. P00750). In aspects of the invention, tPA is decreased in subjects with endometriosis compared to those with other benign conditions.

Urokinase Type Plasminogen Activator (uPA)

One exemplary biomarker present in the panel of the invention is uPA. uPA and its inhibitor, PAI-I, play a key role in tumor invasion and metastasis. They were the first novel tumor biological factors to be validated at the highest level of evidence (LOE I) regarding their clinical utility in breast cancer. Their antigen levels are determined in tumor tissue extracts by standardized, quality-assured immunometric assays (ELISA). Since the late 1980s, numerous independent studies have demonstrated that patients with low levels of uPA and PAI-I in their primary tumor tissue have a significantly better survival than patients with high levels of either factor. uPA is a 431 amino acid protein (UniProt Accession No. P00749). In aspects of the invention, uPA is increased in subjects with endometriosis compared to those with other benign conditions.

Urokinase Type Plasminogen Activator Receptor (uPAR, CD87)

One exemplary biomarker present in the panel of the invention is uPAR. uPAR is involved in plasminogen activation, and is involved in the regulation of proteolysis. It is also found highly expressed in many malignant tumors. uPAR is a 335 amino acid protein (UniProt Accession No. Q03405). In aspects of the invention, uPAR is decreased in subjects with endometriosis compared to those with other benign conditions.

### Vascular Endothelial Growth Factor (VEGF)

One exemplary biomarker present in the panel of the invention is VEGF. VEGF is a potent angiogenic factor and was first described as an essential growth factor for vascular endothelial cells. VEGF is up-regulated in many tumors and its contribution to tumor angiogenesis is well defined. In addition to endothelial cells, VEGF and VEGF receptors are expressed on numerous non-endothelial cells including tumor cells. VEGF is a 232 amino acid protein (VEGFA UniProt Accession No. P15692). In aspects of the invention, VEGF is decreased in subjects with endometriosis compared to those with other benign conditions.

### von Willebrand Factor (VWF)

One exemplary biomarker present in the panel of the invention is VWF. VWF is a blood glycoprotein that is required for normal hemostasis, and deficiency of VWF, or von Willebrand disease (VWD), is the most common inherited bleeding disorder. VWF mediates the adhesion of platelets to sites of vascular damage by binding to specific platelet membrane glycoproteins and to constituents of exposed connective tissue. These activities appear to be regulated by allosteric mechanisms and possibly by hydrodynamic shear forces. VWF also is a carrier protein for blood clotting factor VIII, and this interaction is required for normal factor VIII survival in the circulation. VWF is a 2813 amino acid protein (UniProt Accession No. P04275). In aspects of the invention, VWF is decreased in subjects with endometriosis compared to those with other benign conditions.

### YKL-40 (CHI3L)

One exemplary biomarker present in the panel of the invention is YKL-40. YKL-40 is expressed and secreted by macrophages, neutrophils, fibroblast-like synovial cells, chondrocytes, vascular smooth muscle cells and hepatic stellate cells. Its pattern of expression is associated with pathogenic processes related to inflammation, extracellular tissue remodeling, fibrosis and solid carcinomas. It is assumed that YKL-40 plays a role in cancer cell proliferation, survival, invasiveness and in the regulation of cell-matrix interactions. YKL-40 is a 383 amino acid protein (UniProt Accession No. P36222). In aspects of the invention, YKL-40 is decreased in subjects with endometriosis compared to those with other benign conditions.

### Apolipoprotein A1 (ApoA1)

One exemplary biomarker present in the panel of the invention is ApoA1. ApoA1 is a 267 amino acid protein (UniProt Accession No. P02647). The amino acid sequence of an exemplary ApoA1 polypeptide is set forth in FIG. 63. Antibodies to Apolipoprotein A1 can be made using any method well known in the art, or can be purchased from, for example, Santa Cruz Biotechnology, Inc. (Catalog Number sc-130503) (www.scbt.com, Santa Cruz, Calif.). In aspects of the invention, ApoA1 is altered/slightly decreased in subjects with endometriosis compared to those with other benign conditions.

### Transferrin (TRF)

One exemplary biomarker present in the panel of the invention is TRF. TRF is a 698 amino acid protein (UniProt Accession No. P02787). The amino acid sequence of an exemplary TRF polypeptide is set forth in FIG. 63. Antibodies to transferrin can be made using any method well known in the art, or can be purchased from, for example, Santa Cruz Biotechnology, Inc. (Catalog Number sc-52256) (www.scbt.com, Santa Cruz, Calif.). In aspects of the invention, TRF is altered in subjects with endometriosis compared to those with other benign conditions.

### Transthyretin (TT)/Prealbumin (PREA)

One exemplary biomarker present in the panel of the invention is transthyretin. TT is a 147 amino acid protein (UniProt Accession No. P02766). The amino acid sequence of an exemplary TT polypeptide is set forth in FIG. 63. Antibodies to transthyretin can be made using any method well known in the art, or can be purchased from, for example, Santa Cruz Biotechnology, Inc. (Catalog Number sc-13098) (www.scbt.com, Santa Cruz, Calif.). In aspects of the invention, TT is altered in subjects with endometriosis compared to those with other benign conditions.

### Human Epididymis Protein 4 (HE4, WFDC2)

One exemplary biomarker present in the panel of the invention is HE4. HE4 is a 124 amino acid protein (UniProt Accession No. Q14508). The amino acid sequence of an exemplary HE4 polypeptide is set forth in FIG. 63. Antibodies to HE4 can be made using any method well known in the art, or can be purchased from, for example, Santa Cruz Biotechnology, Inc. (Catalog Number sc-27570) (www.scbt.com, Santa Cruz, Calif.). In aspects of the invention, HE4 is altered in subjects with endometriosis compared to those with other benign conditions.

### Biomarkers and Different Forms of a Protein

Proteins frequently exist in a sample in a plurality of different forms. These forms can result from pre- and/or post-translational modification. Pre-translational modified forms include allelic variants, splice variants and RNA editing forms. Post-translationally modified forms include forms resulting from proteolytic cleavage (e.g., cleavage of a signal sequence or fragments of a parent protein), glycosylation, phosphorylation, lipidation, oxidation, methylation, cysteinylation, sulfonation and acetylation. When detecting or measuring a protein in a sample, any or all of the forms may be measured to determine the level of biomarker or a form of interest is measured. The ability to differentiate between different forms of a protein depends upon the nature of the difference and the method used to detect or measure the protein. For example, an immunoassay using a monoclonal antibody will detect all forms of a protein containing the epitope and will not distinguish between them. However, a sandwich immunoassay that uses two antibodies directed against different epitopes on a protein will detect all forms of the protein that contain both epitopes and will not detect those forms that contain only one of the epitopes. Distinguishing different forms of an analyte or specifically detecting a particular form of an analyte is referred to as “resolving” the analyte.

Mass spectrometry is a particularly powerful methodology to resolve different forms of a protein because the different forms typically have different masses that can be resolved by mass spectrometry. Accordingly, if one form of a protein is a superior biomarker for a disease than another form of the biomarker, mass spectrometry may be able to specifically detect and measure the useful form where traditional immunoassay fails to distinguish the forms and fails to specifically detect to useful biomarker.

One useful methodology combines mass spectrometry with immunoassay. For example, a biospecific capture reagent (e.g., an antibody, aptamer, Affibody, and the like that recognizes the biomarker and other forms of it) is used to capture the biomarker of interest. In embodiments, the biospecific capture reagent is bound to a solid phase, such as a bead, a plate, a membrane or an array. After unbound materials are washed away, the captured analytes are detected and/or measured by mass spectrometry. This method will also result in the capture of protein interactors that are bound to the proteins or that are otherwise recognized by antibodies and that, themselves, can be biomarkers. Various forms of mass spectrometry are useful for detecting the protein forms, including laser desorption approaches, such as traditional MALDI or SELDI, electrospray ionization, and the like.

Thus, when reference is made herein to detecting a particular protein or to measuring the amount of a particular protein, it means detecting and measuring the protein with or without resolving various forms of protein. For example, the step of “detecting β-2 microglobulin” includes measuring β-2 microglobulin by means that do not differentiate between various forms of the protein (e.g., certain immunoassays) as well as by means that differentiate some forms from other forms or that measure a specific form of the protein.

### Detection of Biomarkers for Endometriosis

The biomarkers of this invention can be detected by any suitable method. The methods described herein can be used individually or in combination for a more accurate detection of the biomarkers (e.g., biochip in combination with mass spectrometry, immunoassay in combination with mass spectrometry, and the like).

Detection paradigms that can be employed in the invention include, but are not limited to, optical methods, electrochemical methods (voltammetry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

These and additional methods are described infra.

### Detection by Immunoassay

In particular embodiments, the biomarkers of the invention are measured by immunoassay. Immunoassay typically utilizes an antibody (or other agent that specifically binds the marker) to detect the presence or level of a biomarker in a sample. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

This invention contemplates traditional immunoassays including, for example, Western blot, sandwich immunoassays including ELISA and other enzyme immunoassays, fluorescence-based immunoassays, and chemiluminescence. Nephelometry is an assay done in liquid phase, in which antibodies are in solution. Binding of the antigen to the antibody results in changes in absorbance, which is measured. Other forms of immunoassay include magnetic immunoassay, radioimmunoassay, and real-time immunoquantitative PCR (iqPCR).

Immunoassays can be carried out on solid substrates (e.g., chips, beads, microfluidic platforms, membranes) or on any other forms that supports binding of the antibody to the marker and subsequent detection. A single marker may be detected at a time or a multiplex format may be used. Multiplex immunoanalysis may involve planar microarrays (protein chips) and bead-based microarrays (suspension arrays).

In a SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

### Detection by Biochip

In aspects of the invention, a sample is analyzed by means of a biochip (also known as a microarray). The polypeptides and nucleic acid molecules of the invention are useful as hybridizable array elements in a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3. i-e3. vii, 2000), MacBeath et al., (Science 289:1760-1763, 2000), Zhu et al. (Nature Genet. 26:283-289), and in U.S. Pat. No. 6,436,665, hereby incorporated by reference.

### Detection by Protein Biochip

In aspects of the invention, a sample is analyzed by means of a protein biochip (also known as a protein microarray). Such biochips are useful in high-throughput low-cost screens to identify alterations in the expression or post-translation modification of a polypeptide of the invention, or a fragment thereof. In embodiments, a protein biochip of the invention binds a biomarker present in a subject sample and detects an alteration in the level of the biomarker. Typically, a protein biochip features a protein, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., antibodies that bind a marker of the invention) are spotted on a substrate using any convenient method known to the skilled artisan (e.g., by hand or by inkjet printer).

In embodiments, the protein biochip is hybridized with a detectable probe. Such probes can be polypeptide, nucleic acid molecules, antibodies, or small molecules. For some applications, polypeptide and nucleic acid molecule probes are derived from a biological sample taken from a patient, such as a bodily fluid (such as blood, blood serum, plasma, saliva, urine, ascites, cyst fluid, and the like); a homogenized tissue sample (e.g., a tissue sample obtained by biopsy); or a cell isolated from a patient sample. Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

Many protein biochips are described in the art. These include, for example, protein biochips produced by Ciphergen Biosystems, Inc. (Fremont, Calif.), Zyomyx (Hayward, Calif.), Packard BioScience Company (Meriden, Conn.), Phylos (Lexington, Mass.), Invitrogen (Carlsbad, Calif.), Biacore (Uppsala, Sweden) and Procognia (Berkshire, UK). Examples of such protein biochips are described in the following patents or published patent applications: U.S. Pat. Nos. 6,225,047; 6,537,749; 6,329,209; and 5,242,828; PCT International Publication Nos. WO 00/56934; WO 03/048768; and WO 99/51773.

### Detection by Nucleic Acid Biochip

In aspects of the invention, a sample is analyzed by means of a nucleic acid biochip (also known as a nucleic acid microarray). To produce a nucleic acid biochip, oligonucleotides may be synthesized or bound to the surface of a substrate using a chemical coupling procedure and an ink jet application apparatus, as described in PCT application WO95/251116 (Baldeschweiler et al.). Alternatively, a gridded array may be used to arrange and link cDNA fragments or oligonucleotides to the surface of a substrate using a vacuum system, thermal, UV, mechanical or chemical bonding procedure.

A nucleic acid molecule (e.g. RNA or DNA) derived from a biological sample may be used to produce a hybridization probe as described herein. The biological samples are generally derived from a patient, e.g., as a bodily fluid (such as blood, blood serum, plasma, saliva, urine, ascites, cyst fluid, and the like); a homogenized tissue sample (e.g., a tissue sample obtained by biopsy); or a cell isolated from a patient sample. For some applications, cultured cells or other tissue preparations may be used. The mRNA is isolated according to standard methods, and cDNA is produced and used as a template to make complementary RNA suitable for hybridization. Such methods are well known in the art. The RNA is amplified in the presence of fluorescent nucleotides, and the labeled probes are then incubated with the microarray to allow the probe sequence to hybridize to complementary oligonucleotides bound to the biochip.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In embodiments, hybridization will occur at 37° C. in 500 mM NaCl, 50 mM trisodium citrate, 1% SDS, 35% formamide, and 100 μg/ml denatured salmon sperm DNA (ssDNA). In other embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., of at least about 42° C., or of at least about 68° C. In embodiments, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In other embodiments, wash steps will occur at 68° C. in 15 mM NaCl, 1.5 mM trisodium citrate, and 0.1% SDS. Additional variations on these conditions will be readily apparent to those skilled in the art.

Detection system for measuring the absence, presence, and amount of hybridization for all of the distinct nucleic acid sequences are well known in the art. For example, simultaneous detection is described in Heller et al., Proc. Natl. Acad. Sci. 94:2150-2155, 1997. In embodiments, a scanner is used to determine the levels and patterns of fluorescence.

### Detection by Mass Spectrometry

In aspects of the invention, the biomarkers of this invention are detected by mass spectrometry (MS). Mass spectrometry is a well-known tool for analyzing chemical compounds that employs a mass spectrometer to detect gas phase ions. Mass spectrometers are well known in the art and include, but are not limited to, time-of-flight, magnetic sector, quadrupole filter, ion trap, ion cyclotron resonance, electrostatic sector analyzer and hybrids of these. The method may be performed in an automated (Villanueva, et al., Nature Protocols (2006) 1(2):880-891) or semi-automated format. This can be accomplished, for example with the mass spectrometer operably linked to a liquid chromatography device (LC-MS/MS or LC-MS) or gas chromatography device (GC-MS or GC-MS/MS). Methods for performing mass spectrometry are well known and have been disclosed, for example, in US Patent Application Publication Nos: 20050023454; 20050035286; U.S. Pat. No. 5,800,979 and the references disclosed therein.

### Laser Desorption/Ionization

In embodiments, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer. The analysis of proteins by LDI can take the form of MALDI or of SELDI. The analysis of proteins by LDI can take the form of MALDI or of SELDI.

Laser desorption/ionization in a single time of flight instrument typically is performed in linear extraction mode. Tandem mass spectrometers can employ orthogonal extraction modes.

### Matrix-Assisted Laser Desorption/Ionization (MALDI) and Electrospray Ionization (ESI)

In embodiments, the mass spectrometric technique for use in the invention is matrix-assisted laser desorption/ionization (MALDI) or electrospray ionization (ESI). In related embodiments, the procedure is MALDI with time of flight (TOF) analysis, known as MALDI-TOF MS. This involves forming a matrix on a membrane with an agent that absorbs the incident light strongly at the particular wavelength employed. The sample is excited by UV or IR laser light into the vapor phase in the MALDI mass spectrometer. Ions are generated by the vaporization and form an ion plume. The ions are accelerated in an electric field and separated according to their time of travel along a given distance, giving a mass/charge (m/z) reading which is very accurate and sensitive. MALDI spectrometers are well known in the art and are commercially available from, for example, PerSeptive Biosystems, Inc. (Framingham, Mass., USA).

Magnetic-based serum processing can be combined with traditional MALDI-TOF. Through this approach, improved peptide capture is achieved prior to matrix mixture and deposition of the sample on MALDI target plates. Accordingly, in embodiments, methods of peptide capture are enhanced through the use of derivatized magnetic bead based sample processing.

MALDI-TOF MS allows scanning of the fragments of many proteins at once. Thus, many proteins can be run simultaneously on a polyacrylamide gel, subjected to a method of the invention to produce an array of spots on a collecting membrane, and the array may be analyzed. Subsequently, automated output of the results is provided by using an server (e.g., ExPASy) to generate the data in a form suitable for computers.

Other techniques for improving the mass accuracy and sensitivity of the MALDI-TOF MS can be used to analyze the fragments of protein obtained on a collection membrane. These include, but are not limited to, the use of delayed ion extraction, energy reflectors, ion-trap modules, and the like. In addition, post source decay and MS-MS analysis are useful to provide further structural analysis. With ESI, the sample is in the liquid phase and the analysis can be by ion-trap, TOF, single quadrupole, multi-quadrupole mass spectrometers, and the like. The use of such devices (other than a single quadrupole) allows MS-MS or MSⁿ analysis to be performed. Tandem mass spectrometry allows multiple reactions to be monitored at the same time.

Capillary infusion may be employed to introduce the marker to a desired mass spectrometer implementation, for instance, because it can efficiently introduce small quantities of a sample into a mass spectrometer without destroying the vacuum. Capillary columns are routinely used to interface the ionization source of a mass spectrometer with other separation techniques including, but not limited to, gas chromatography (GC) and liquid chromatography (LC). GC and LC can serve to separate a solution into its different components prior to mass analysis. Such techniques are readily combined with mass spectrometry. One variation of the technique is the coupling of high-performance liquid chromatography (HPLC) to a mass spectrometer for integrated sample separation/and mass spectrometer analysis.

Quadrupole mass analyzers may also be employed as needed to practice the invention. Fourier-transform ion cyclotron resonance (FTMS) can also be used for some invention embodiments. It offers high resolution and the ability of tandem mass spectrometry experiments. FTMS is based on the principle of a charged particle orbiting in the presence of a magnetic field. Coupled to ESI and MALDI, FTMS offers high accuracy with errors as low as 0.001%.

### Surface-Enhanced Laser Desorption/Ionization (SELDI)

In embodiments, the mass spectrometric technique for use in the invention is “Surface Enhanced Laser Desorption and Ionization” or “SELDI,” as described, for example, in U.S. Pat. Nos. 5,719,060 and 6,225,047, both to Hutchens and Yip. This refers to a method of desorption/ionization gas phase ion spectrometry (e.g., mass spectrometry) in which an analyte (here, one or more of the biomarkers) is captured on the surface of a SELDI mass spectrometry probe.

SELDI has also been called “affinity capture mass spectrometry.” It also is called “Surface-Enhanced Affinity Capture” or “SEAC”. This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent can be any material capable of binding an analyte. The capture reagent is attached to the probe surface by physisorption or chemisorption. In certain embodiments the probes have the capture reagent already attached to the surface. In other embodiments, the probes are pre-activated and include a reactive moiety that is capable of binding the capture reagent, e.g., through a reaction forming a covalent or coordinate covalent bond. Epoxide and acyl-imidizole are useful reactive moieties to covalently bind polypeptide capture reagents such as antibodies or cellular receptors. Nitrilotriacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

“Chromatographic adsorbent” refers to an adsorbent material typically used in chromatography. Chromatographic adsorbents include, for example, ion exchange materials, metal chelators (e.g., nitrilotriacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

“Biospecific adsorbent” refers to an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances, the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047. A “bioselective adsorbent” refers to an adsorbent that binds to an analyte with an affinity of at least 10- M.

Protein biochips produced by Ciphergen comprise surfaces having chromatographic or biospecific adsorbents attached thereto at addressable locations. Ciphergen's ProteinChip© arrays include NP20 (hydrophilic); H4 and H50 (hydrophobic); SAX-2, Q-10 and (anion exchange); WCX-2 and CM-10 (cation exchange); IMAC-3, IMAC-30 and IMAC-50 (metal chelate); and PS-10, PS-20 (reactive surface with acyl-imidazole, epoxide) and PG-20 (protein G coupled through acyl-imidazole). Hydrophobic ProteinChip arrays have isopropyl or nonylphenoxy-poly(ethylene glycol)methacrylate functionalities. Anion exchange ProteinChip arrays have quaternary ammonium functionalities. Cation exchange ProteinChip arrays have carboxylate functionalities. Immobilized metal chelate ProteinChip arrays have nitrilotriacetic acid functionalities (IMAC 3 and IMAC 30) or O-methacryloyl-N,N-bis-carboxymethyl tyrosine functionalities (IMAC 50) that adsorb transition metal ions, such as copper, nickel, zinc, and gallium, by chelation. Preactivated ProteinChip arrays have acyl-imidazole or epoxide functional groups that can react with groups on proteins for covalent binding.

Such biochips are further described in: U.S. Pat. No. 6,579,719 (Hutchens and Yip, “Retentate Chromatography,” Jun. 17, 2003); U.S. Pat. No. 6,897,072 (Rich et al., “Probes for a Gas Phase Ion Spectrometer,” May 24, 2005); U.S. Pat. No. 6,555,813 (Beecher et al., “Sample Holder with Hydrophobic Coating for Gas Phase Mass Spectrometer,” Apr. 29, 2003); U.S. Patent Publication No. U.S. 2003-0032043 A1 (Pohl and Papanu, “Latex Based Adsorbent Chip,” Jul. 16, 2002); and PCT International Publication No. WO 03/040700 (Um et al., “Hydrophobic Surface Chip,” May 15, 2003); U.S. Patent Application Publication No. US 2003/-0218130 A1 (Boschetti et al., “Biochips With Surfaces Coated With Polysaccharide-Based Hydrogels,” Apr. 14, 2003) and U.S. Pat. No. 7,045,366 (Huang et al., “Photocrosslinked Hydrogel Blend Surface Coatings” May 16, 2006).

In general, a probe with an adsorbent surface is contacted with the sample for a period of time sufficient to allow the biomarker or biomarkers that may be present in the sample to bind to the adsorbent. After an incubation period, the substrate is washed to remove unbound material. Any suitable washing solutions can be used; preferably, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. Unless the probe has both SEAC and SEND properties (as described herein), an energy absorbing molecule then is applied to the substrate with the bound biomarkers.

In yet another method, one can capture the biomarkers with a solid-phase bound immuno-adsorbent that has antibodies that bind the biomarkers. After washing the adsorbent to remove unbound material, the biomarkers are eluted from the solid phase and detected by applying to a SELDI biochip that binds the biomarkers and analyzing by SELDI.

The biomarkers bound to the substrates are detected in a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

### Methods of the Invention

Panels comprising biomarkers of the invention are used to characterize endometriosis in a subject to determine whether the subject should be seen by a general surgeon or should be evaluated and/or treated by a gynecologist. In other embodiments, a panel of the invention is used to diagnose or stage endometriosis by determining the molecular profile of the endometriosis. In certain embodiments, panels of the invention are used to select a course of treatment for a subject. The phrase “endometriosis status” includes any distinguishable manifestation of the disease, including non-disease. Based on this status, further procedures may be indicated, including additional diagnostic tests or therapeutic procedures or regimens.

In aspects of the invention, the biomarkers of the invention can be used in diagnostic tests to identify early stage endometriosis in a subject.

The correlation of test results with endometriosis involves applying a classification algorithm of some kind to the results to generate the status. The classification algorithm may be as simple as determining whether or not the amounts of the markers listed in Table 1 are above or below a particular cut-off number. When multiple biomarkers are used, the classification algorithm may be a linear regression formula. Alternatively, the classification algorithm may be the product of any of a number of learning algorithms described herein.

In the case of complex classification algorithms, it may be necessary to perform the algorithm on the data, thereby determining the classification, using a computer, e.g., a programmable digital computer. In either case, one can then record the status on tangible medium, for example, in computer-readable format such as a memory drive or disk or simply printed on paper. The result also could be reported on a computer screen.

### Biomarkers of the Invention

Individual biomarkers are useful diagnostic biomarkers. In addition, as described in the examples, it has been found that a specific combination of biomarkers provides greater predictive value of a particular status than any single biomarker alone, or any other combination of previously identified biomarkers. Specifically, the detection of a plurality of biomarkers in a sample can increase the sensitivity, accuracy and specificity of the test.

Each biomarker described herein can be differentially present in endometriosis, and, therefore, each is individually useful in aiding in the determination of endometriosis status. The method involves, first, measuring the selected biomarker in a subject, sample using any method well known in the art, including but not limited to the methods described herein, e.g. capture on a SELDI biochip followed by detection by mass spectrometry and, second, comparing the measurement with a diagnostic amount or cut-off that distinguishes a positive endometriosis status from a negative endometriosis status. The diagnostic amount represents a measured amount of a biomarker above which or below which a subject is classified as having a particular endometriosis status. For example, if the biomarker is up-regulated compared to normal during endometriosis, then a measured amount above the diagnostic cutoff provides a diagnosis of endometriosis. Alternatively, if the biomarker is down-regulated during endometriosis, then a measured amount below the diagnostic cutoff provides a diagnosis of endometriosis. As is well understood in the art, by adjusting the particular diagnostic cut-off used in an assay, one can increase sensitivity or specificity of the diagnostic assay depending on the preference of the diagnostician. The particular diagnostic cut-off can be determined, for example, by measuring the amount of the biomarker in a statistically significant number of samples from subjects with the different endometriosis statuses, as was done here, and drawing the cut-off to suit the diagnostician's desired levels of specificity and sensitivity.

The biomarkers of this invention (used alone or in combination) show a statistical difference in different endometriosis statuses of at least p≤0.05, p≤10⁻², p≤10⁻³, p≤10⁻⁴, or p≤10⁻⁵. Diagnostic tests that use these biomarkers alone or in combination show a sensitivity and specificity of at least 75%, at least 80%, at least 85%, at least 90%, at least 95%, at least 98%, or about 100%.

### Determining Course (Progression/Remission) of Disease

In one embodiment, this invention provides methods for determining the course of disease in a subject. Disease course refers to changes in disease status over time, including disease progression (worsening) and disease regression (improvement). Over time, the amounts or relative amounts (e.g., the pattern) of the biomarkers change. Accordingly, this method involves measuring the panel of biomarkers in a subject at at least two different time points, e.g., a first time and a second time, and comparing the change in amounts, if any. The course of disease (e.g., during treatment) is determined based on these comparisons.

### Reporting the Status

Additional embodiments of the invention relate to the communication of assay results or diagnoses or both to technicians, physicians or patients, for example. In certain embodiments, computers will be used to communicate assay results or diagnoses or both to interested parties, e.g., physicians and their patients. In some embodiments, the assays will be performed or the assay results analyzed in a country or jurisdiction which differs from the country or jurisdiction to which the results or diagnoses are communicated.

In a preferred embodiment of the invention, a diagnosis based on the differential presence or absence in a test subject of the biomarkers of Table 1 is communicated to the subject as soon as possible after the diagnosis is obtained. The diagnosis may be communicated to the subject by the subject's treating physician. Alternatively, the diagnosis may be sent to a test subject by email or communicated to the subject by phone. A computer may be used to communicate the diagnosis by email or phone. In certain embodiments, the message containing results of a diagnostic test may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

### Subject Management

In certain embodiments, the methods of the invention involve managing subject treatment based on the status. Such management includes referral, for example, to a gynecologic specialist. In one embodiment, if a physician makes a diagnosis of endometriosis, then a certain regime of treatment, such as prescription or administration of therapeutic agent (e.g., GnRH agonist/antagonist) might follow. Alternatively, a diagnosis of non-endometriosis might be followed with further testing to determine a specific disease that might the patient might be suffering from. Also, if the diagnostic test gives an inconclusive result on endometriosis status, further tests may be called for.

Additional embodiments of the invention relate to the communication of assay results or diagnoses or both to technicians, physicians or patients, for example. In certain embodiments, computers will be used to communicate assay results or diagnoses or both to interested parties, e.g., physicians and their patients. In some embodiments, the assays will be performed, or the assay results analyzed in a country or jurisdiction which differs from the country or jurisdiction to which the results or diagnoses are communicated.

### Hardware and Software

The any of the methods described herein, the step of correlating the measurement of the biomarker(s) with endometriosis can be performed on general-purpose or specially-programmed hardware or software.

In aspects, the analysis is performed by a software classification algorithm. The analysis of analytes by any detection method well known in the art, including, but not limited to the methods described herein, will generate results that are subject to data processing. Data processing can be performed by the software classification algorithm. Such software classification algorithms are well known in the art and one of ordinary skill can readily select and use the appropriate software to analyze the results obtained from a specific detection method.

In aspects, the analysis is performed by a computer-readable medium. The computer-readable medium can be non-transitory and/or tangible. For example, the computer readable medium can be volatile memory (e.g., random access memory and the like) or non-volatile memory (e.g., read-only memory, hard disks, floppy discs, magnetic tape, optical discs, paper table, punch cards, and the like).

For example, analysis of analytes by time-of-flight mass spectrometry generates a time-of-flight spectrum. The time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. Exemplary software includes, but is not limited to, Ciphergen's ProteinChip© software, in which data processing typically includes TOF-to-M/Z transformation to generate a mass spectrum, baseline subtraction to eliminate instrument offsets and high frequency noise filtering to reduce high frequency noise.

Data generated by desorption and detection of biomarkers can be analyzed with the use of a programmable digital computer. The computer program analyzes the data to indicate the number of biomarkers detected, and optionally the strength of the signal and the determined molecular mass for each biomarker detected. Data analysis can include steps of determining signal strength of a biomarker and removing data deviating from a predetermined statistical distribution. For example, the observed peaks can be normalized, by calculating the height of each peak relative to some reference. The reference can be background noise generated by the instrument and chemicals such as the energy absorbing molecule which is set at zero in the scale.

The computer can transform the resulting data into various formats for display. The standard spectrum can be displayed, but in one useful format only the peak height and mass information are retained from the spectrum view, yielding a cleaner image and enabling biomarkers with nearly identical molecular weights to be more easily seen. In another useful format, two or more spectra are compared, conveniently highlighting unique biomarkers and biomarkers that are up- or down-regulated between samples. Using any of these formats, one can readily determine whether a particular biomarker is present in a sample.

Analysis generally involves the identification of peaks in the spectrum that represent signal from an analyte. Peak selection can be done visually, but software is available, for example, as part of Ciphergen's ProteinChip© software package, that can automate the detection of peaks. This software functions by identifying signals having a signal-to-noise ratio above a selected threshold and labeling the mass of the peak at the centroid of the peak signal. In embodiments, many spectra are compared to identify identical peaks present in some selected percentage of the mass spectra. One version of this software clusters all peaks appearing in the various spectra within a defined mass range and assigns a mass (N/Z) to all the peaks that are near the mid-point of the mass (M/Z) cluster.

In aspects, software used to analyze the data can include code that applies an algorithm to the analysis of the results (e.g., signal to determine whether the signal represents a peak in a signal that corresponds to a biomarker according to the present invention). The software also can subject the data regarding observed biomarker peaks to classification tree or ANN analysis, to determine whether a biomarker peak or combination of biomarker peaks is present that indicates the status of the particular clinical parameter under examination. Analysis of the data may be “keyed” to a variety of parameters that are obtained, either directly or indirectly, from the mass spectrometric analysis of the sample. These parameters include, but are not limited to, the presence or absence of one or more peaks, the shape of a peak or group of peaks, the height of one or more peaks, the log of the height of one or more peaks, and other arithmetic manipulations of peak height data.

### Classification Algorithms for Qualifying Endometriosis Status

In some embodiments, data derived from the assays (e.g., ELISA assays) that are generated using samples such as “known samples” can then be used to “train” a classification model. A “known sample” is a sample that has been pre-classified. The data that are derived from the spectra and are used to form the classification model can be referred to as a “training data set.” Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased versus non-diseased).

The training data set that is used to form the classification model may comprise raw data or pre-processed data. In some embodiments, raw data can be obtained directly from time-of-flight spectra or mass spectra, and then may be optionally “pre-processed” as described above.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines).

In embodiments, a supervised classification method is a recursive partitioning process. Recursive partitioning processes use recursive partitioning trees to classify spectra derived from unknown samples. Further details about recursive partitioning processes are provided in U.S. Patent Application No. 2002 0138208 A1 to Paulse et al., “Method for analyzing mass spectra.”

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. Patent Application No. 2002 0193950 A1 (Gavin et al., “Method or analyzing mass spectra”), U.S. Patent Application No. 2003 0004402 A1 (Hitt et al., “Process for discriminating between biological states based on hidden patterns from biological data”), and U.S. Patent Application No. 2003 0055615 A1 (Zhang and Zhang, “Systems and methods for processing biological expression data”).

The classification models can be formed on and used on any suitable digital computer. Suitable digital computers include micro, mini, or large computers using any standard or specialized operating system, such as a Unix, Windows™ or Linux™ based operating system. The digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

The learning algorithms described above are useful both for developing classification algorithms for the biomarkers already discovered, or for finding new biomarkers for endometriosis. The classification algorithms, in turn, form the base for diagnostic tests by providing diagnostic values (e.g., cut-off points) for biomarkers used singly or in combination.

### Kits for Detection of Biomarkers for Endometriosis

In another aspect, the invention provides kits for aiding in the diagnosis of endometriosis (e.g., identifying endometriosis status, detecting endometriosis, identifying stage of endometriosis, selecting a treatment method for a subject at risk of having endometriosis, and the like), which kits are used to detect biomarkers according to the invention. In one embodiment, the kit comprises agents that specifically recognize the biomarkers identified in Table 1. In related embodiments, the agents are antibodies. The kit may contain 1, 2, 3, 4, 5, or more different antibodies that each specifically recognize one of the biomarkers set forth in Table 1.

In another embodiment, the kit comprises a solid support, such as a chip, a microtiter plate or a bead or resin having capture reagents attached thereon, where the capture reagents bind the biomarkers of the invention. Thus, for example, the kits of the present invention can comprise mass spectrometry probes for SELDI, such as ProteinChip© arrays. In the case of biospecific capture reagents, the kit can comprise a solid support with a reactive surface, and a container comprising the biospecific capture reagents.

The kit can also comprise a washing solution or instructions for making a washing solution, in which the combination of the capture reagent and the washing solution allows capture of the biomarker or biomarkers on the solid support for subsequent detection by, e.g., mass spectrometry. The kit may include more than type of adsorbent, each present on a different solid support.

In a further embodiment, such a kit can comprise instructions for use in any of the methods described herein. In embodiments, the instructions provide suitable operational parameters in the form of a label or separate insert. For example, the instructions may inform a consumer about how to collect the sample, how to wash the probe or the particular biomarkers to be detected.

In yet another embodiment, the kit can comprise one or more containers with controls (e.g., biomarker samples) to be used as standard(s) for calibration.

### EXAMPLES

### Example 1: Serum Proteins Involved in Endometriosis

In order to determine serum biomarkers involved in endometriosis, a cohort of patients having adnexal masses was analyzed. The total cohort size for the analysis was 501 subjects and was taken from the Correlogic database (described in Example 6). Of those, 53 subjects were described as having endometriosis, that is, having at least one of the following: endometriosis, endometriotic cysts, endometrioma, or another benign condition of the endometrium. 298 subjects had another, non-endometriosis, benign condition. 145 subjects had an ovarian malignancy without endometriosis and were excluded from the analysis. Five (5) subjects did not have pathology data and were also excluded from the analysis.

Of the 53 subjects with endometriosis, six (6) subjects were also diagnosed with ovarian cancer. Five (5) subjects were diagnosed with a non-ovarian malignancy, one (1) of which was marked as having both ovarian cancer and a non-ovarian malignancy. These subjects were included in the analysis. Non-ovarian malignancies for subjects without endometriosis were disregarded when placing a subject in the ‘other benign’ cohort.

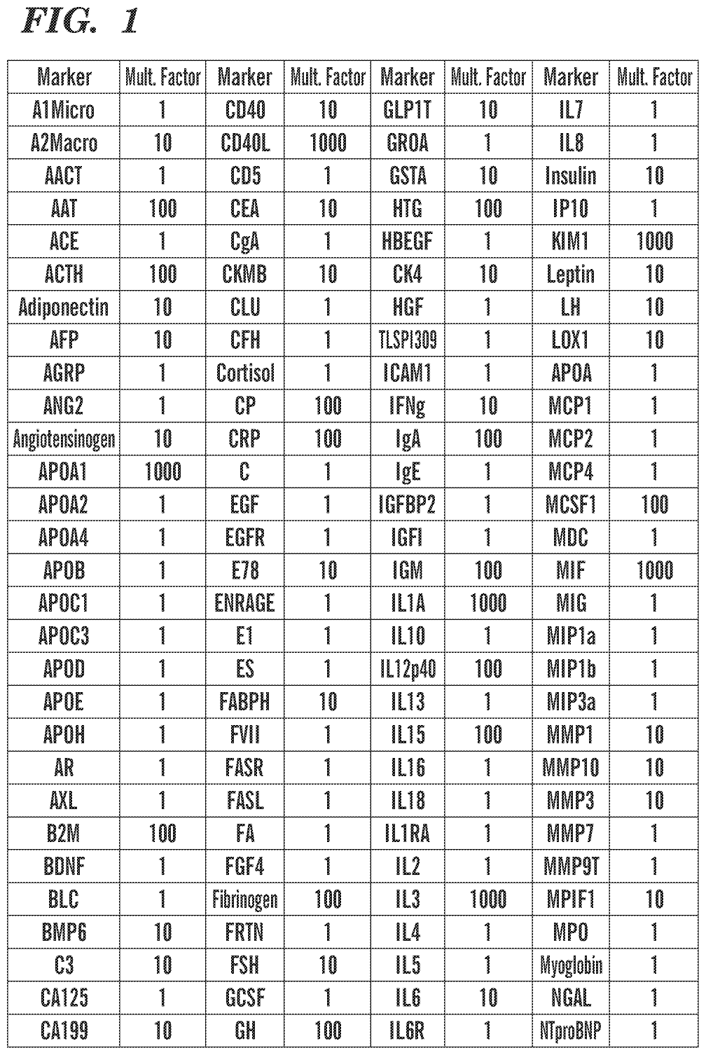

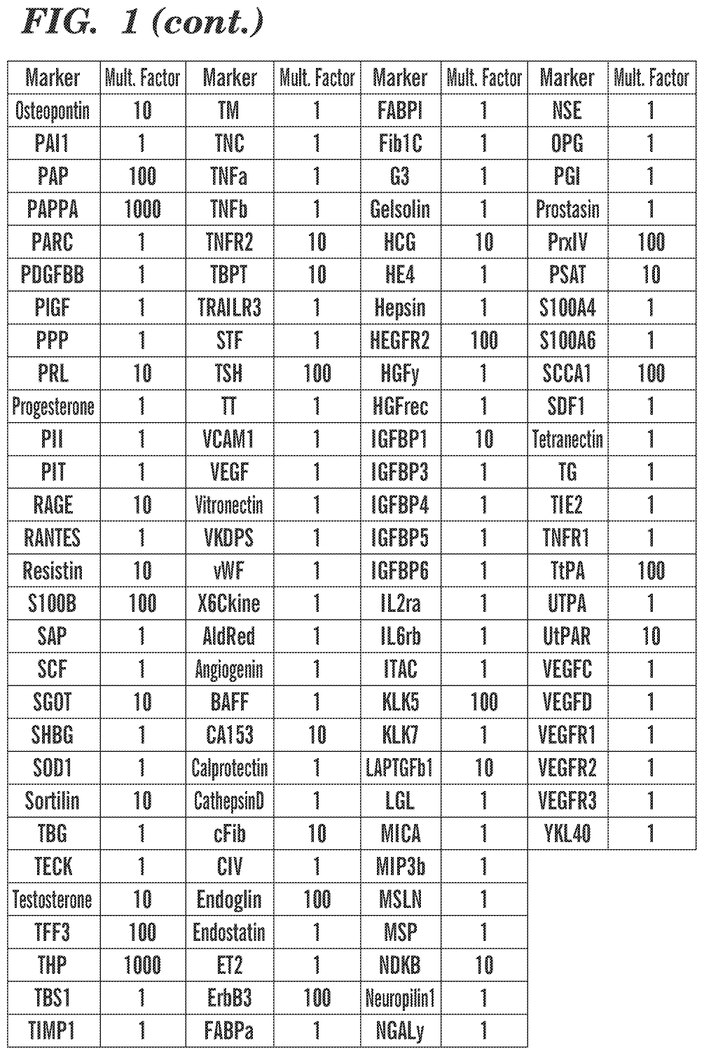

For analysis, biomarker levels reported as <LOW> were substituted with NAs. Levels reported as >[number] were substituted with the number. All plots presented herein reflect biomarker levels that have been normalized using a log 10 transformation. Markers with levels below 1.0 were multiplied as necessary to ensure the minimum was ≥1.0 using the multiplication factors found in FIG. 1.

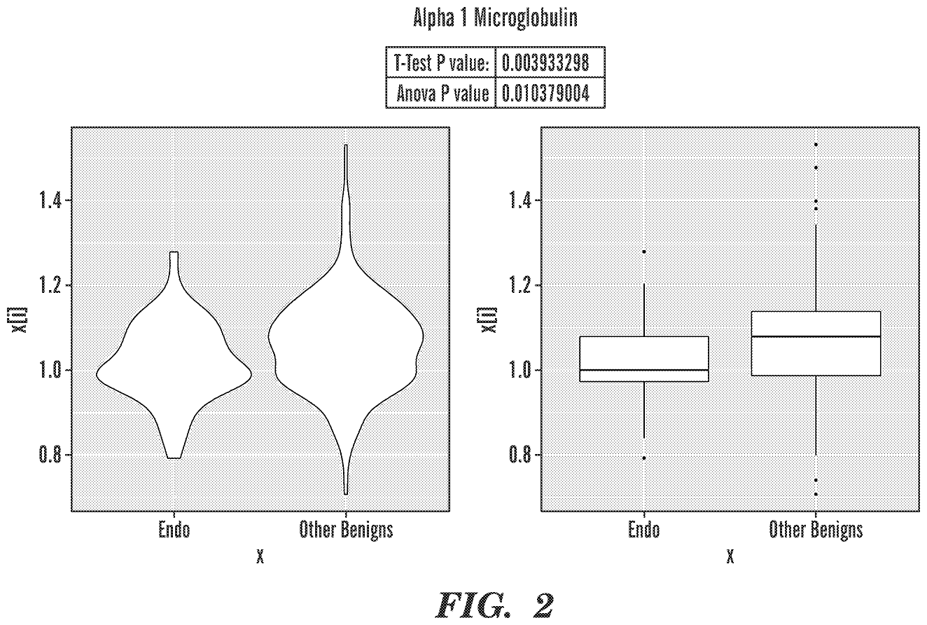

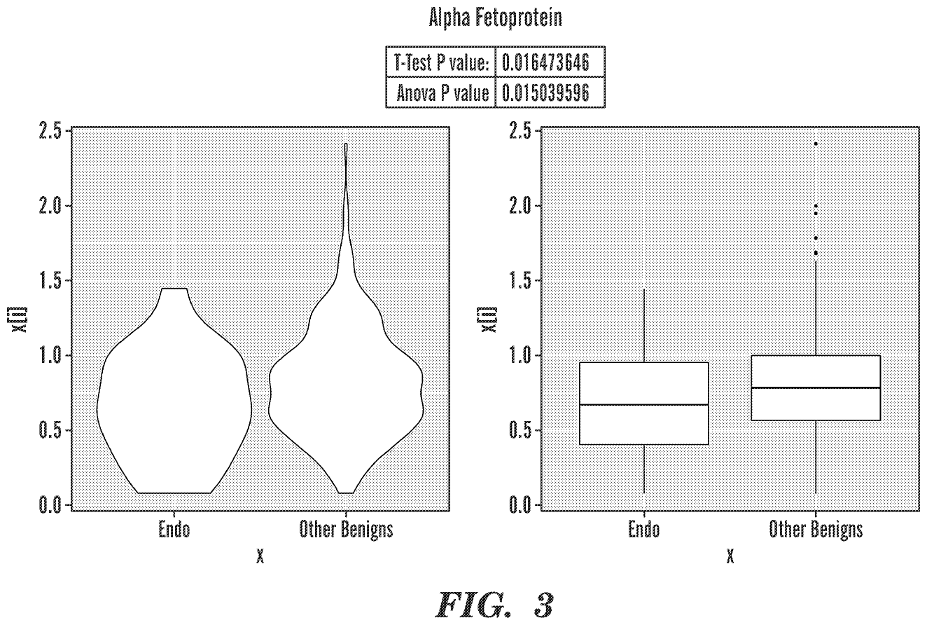

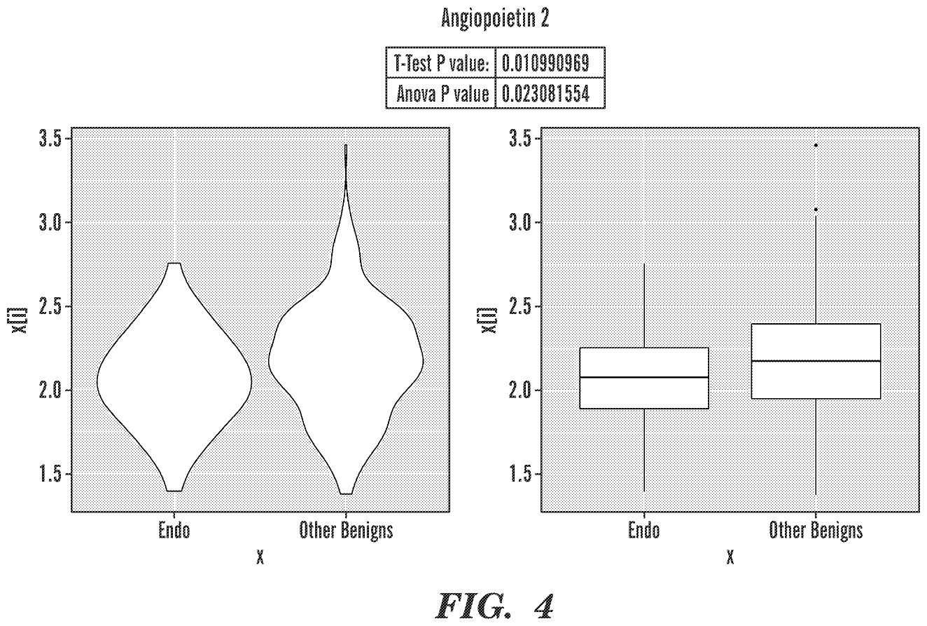

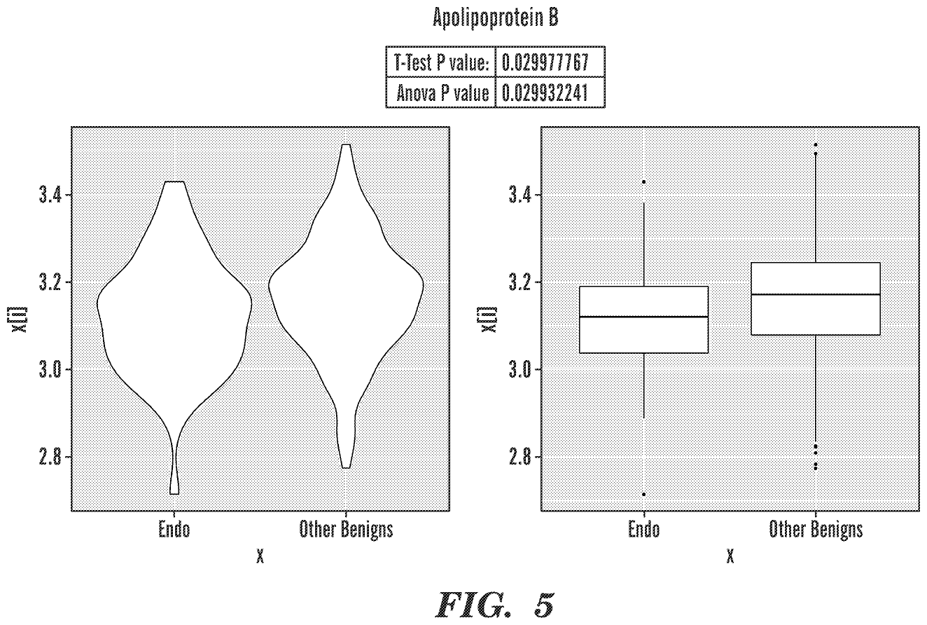

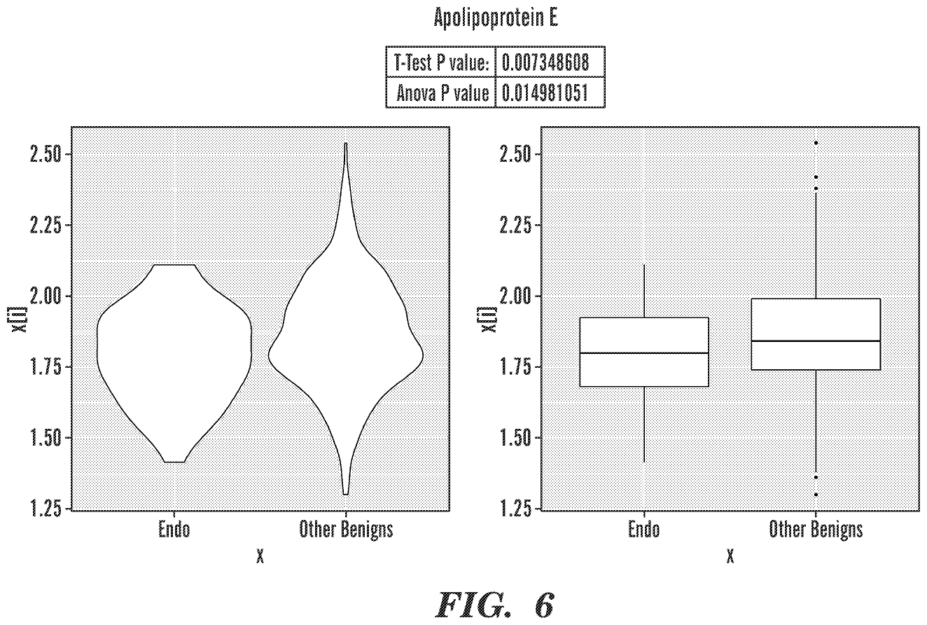

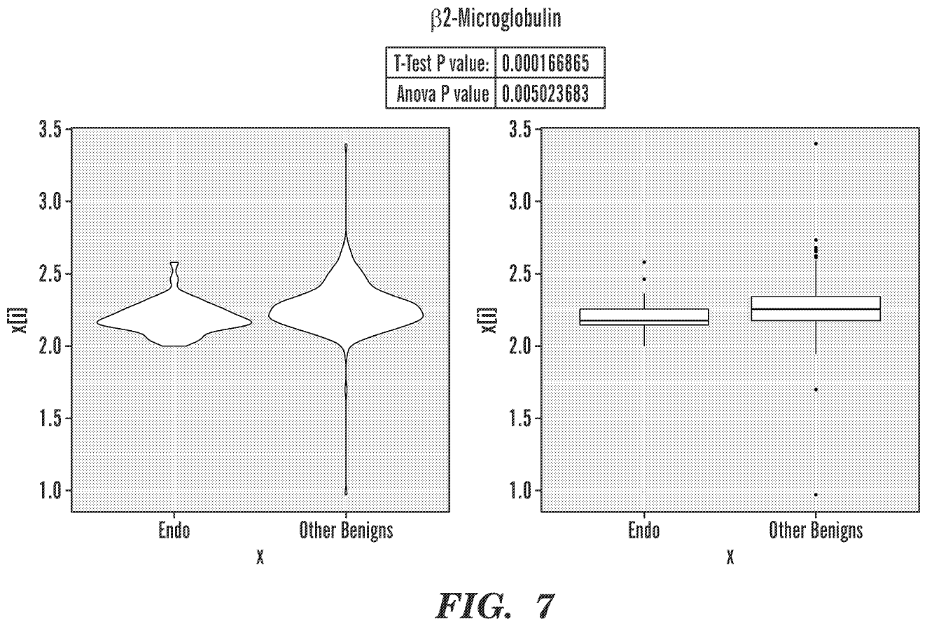

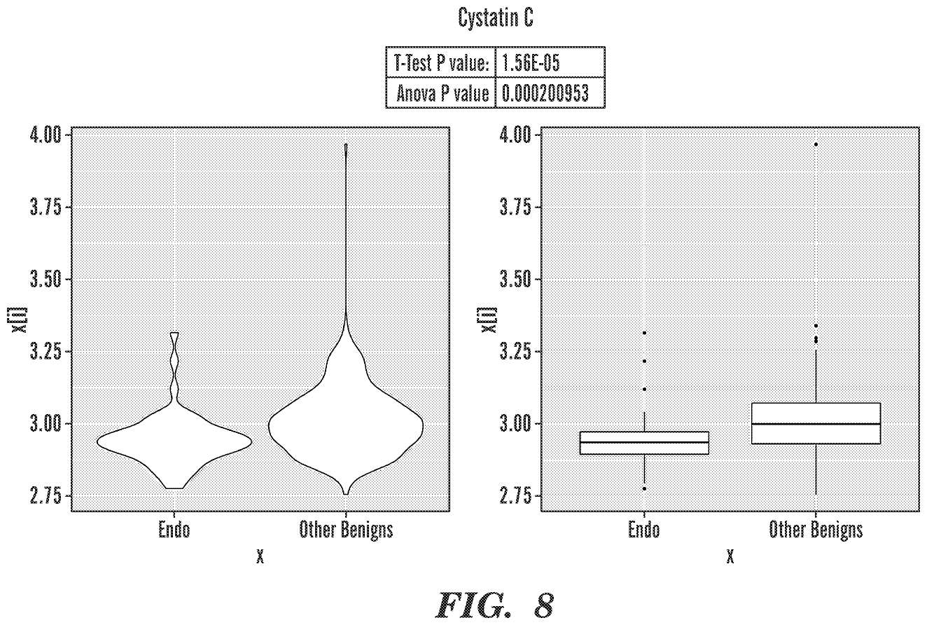

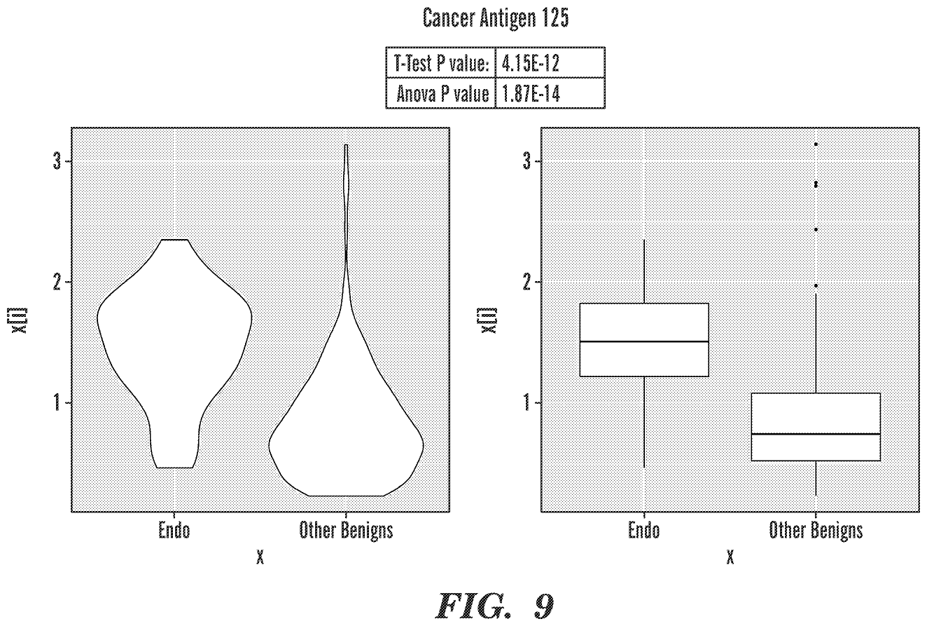

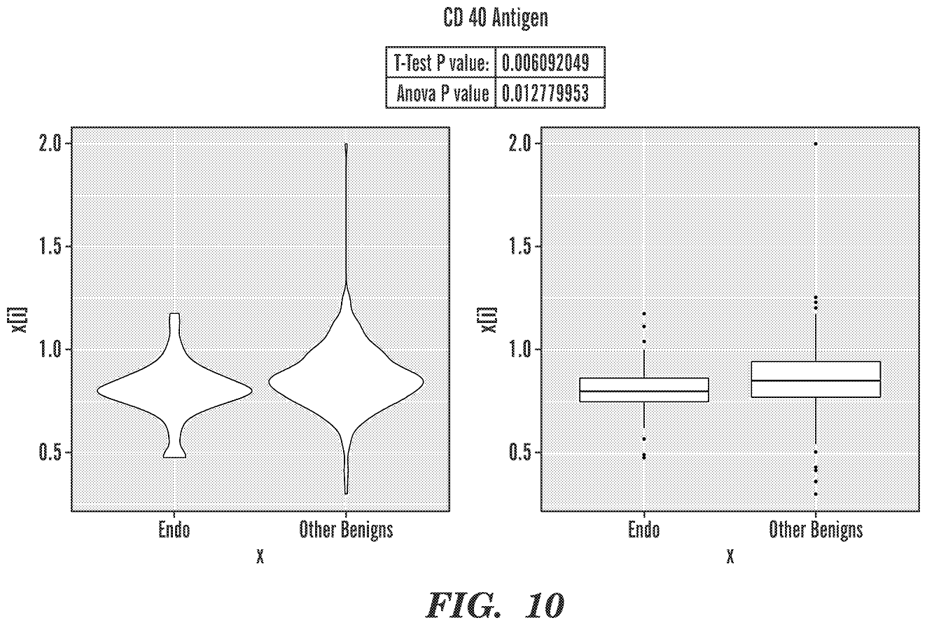

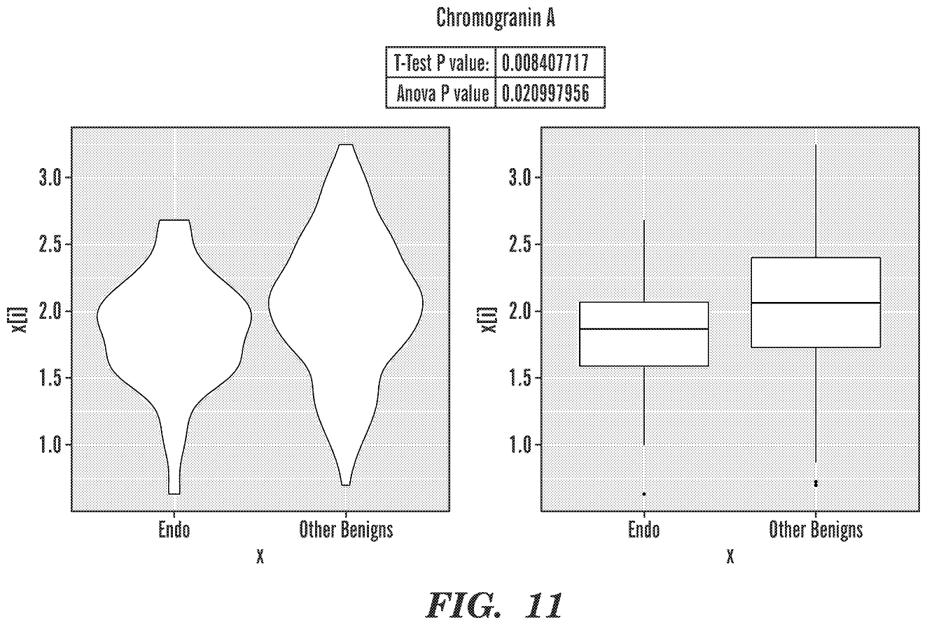

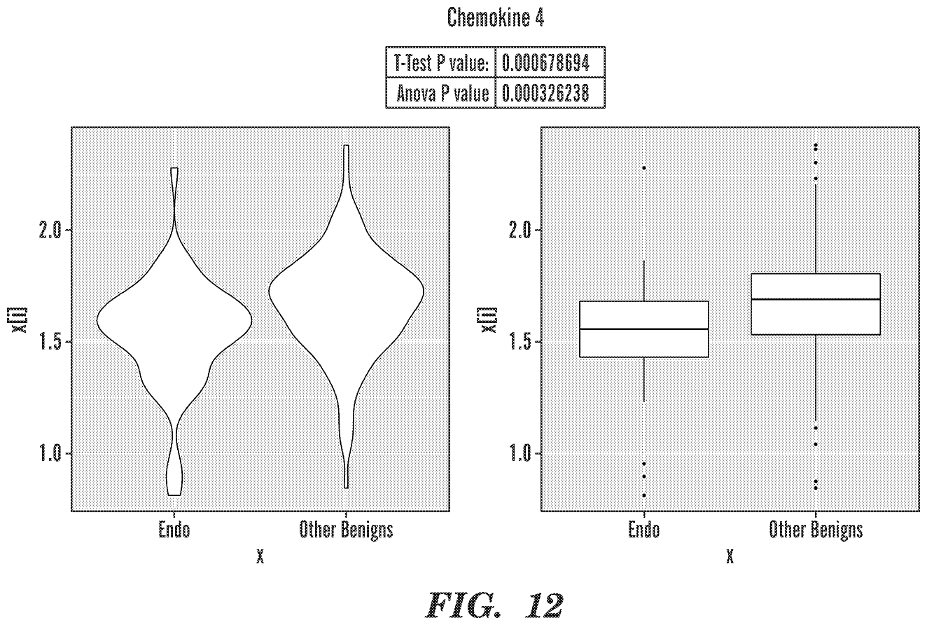

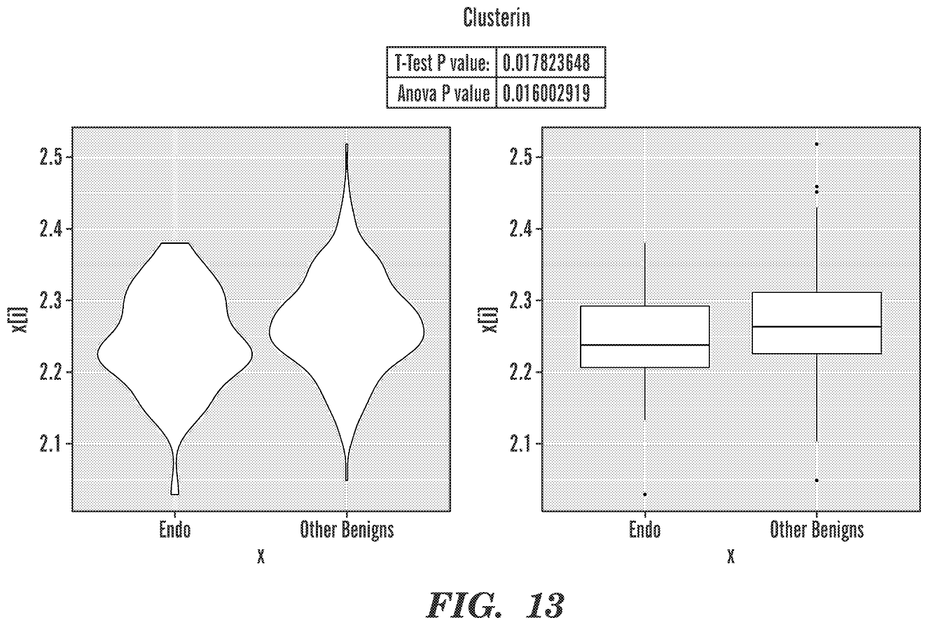

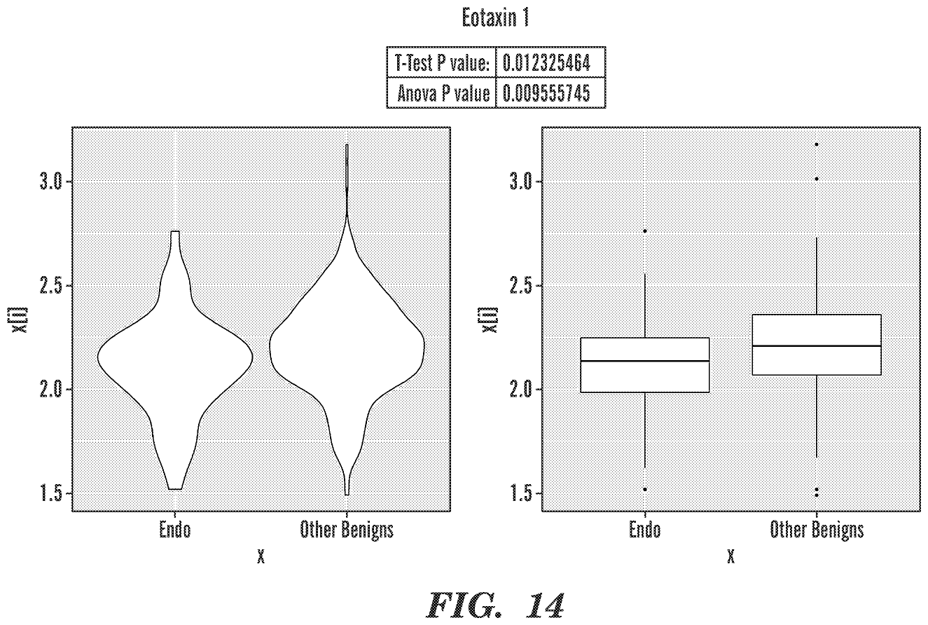

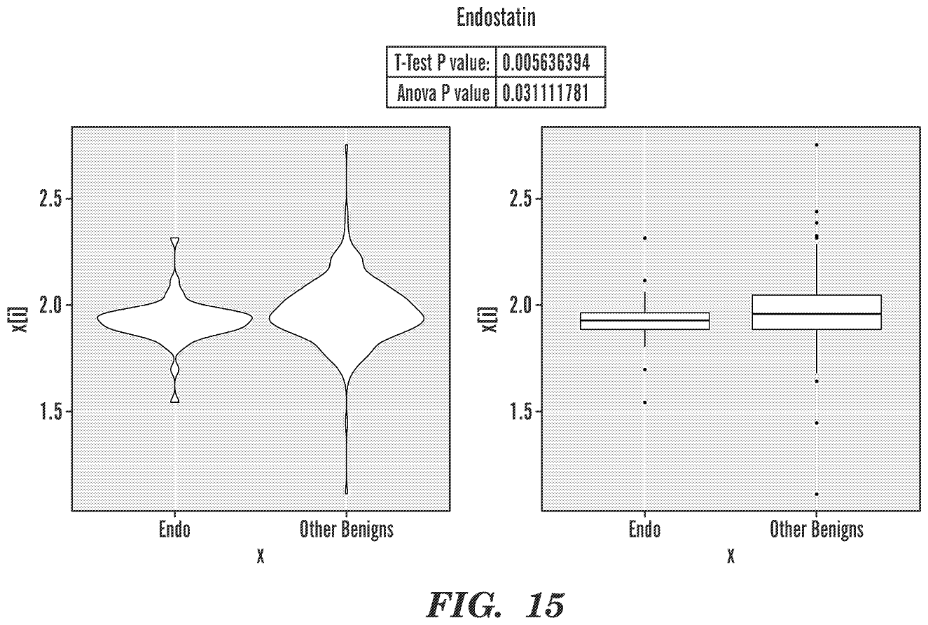

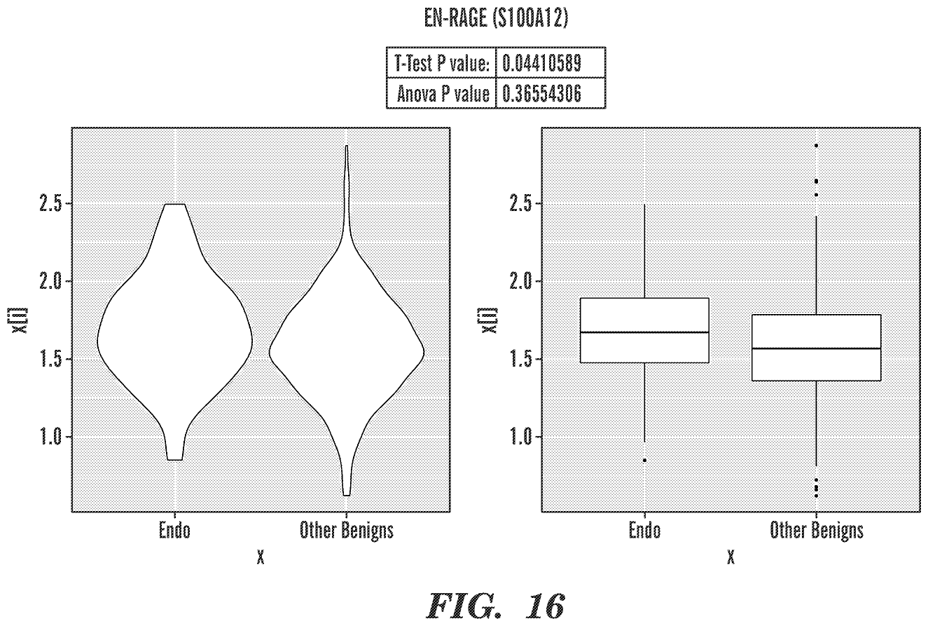

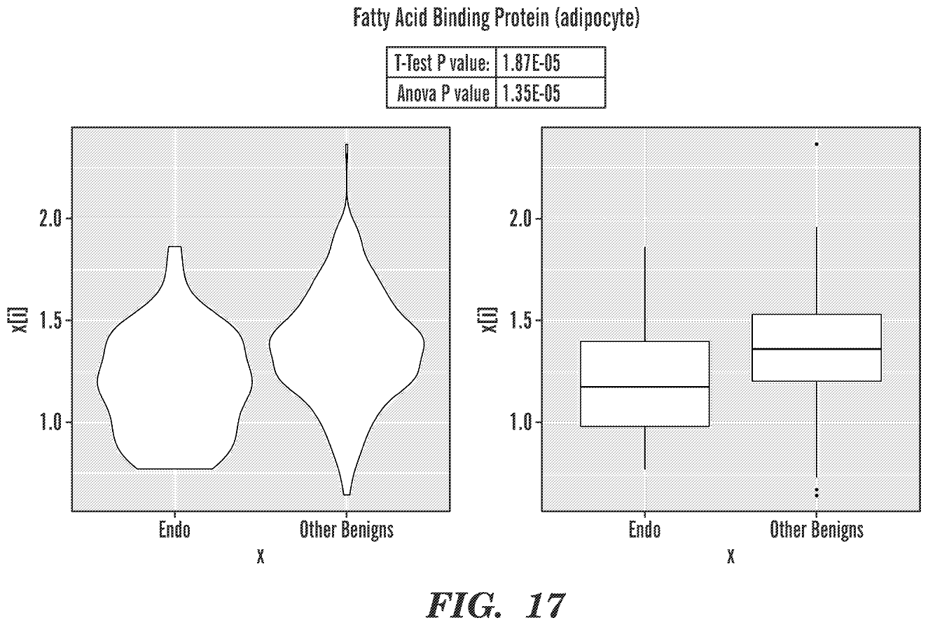

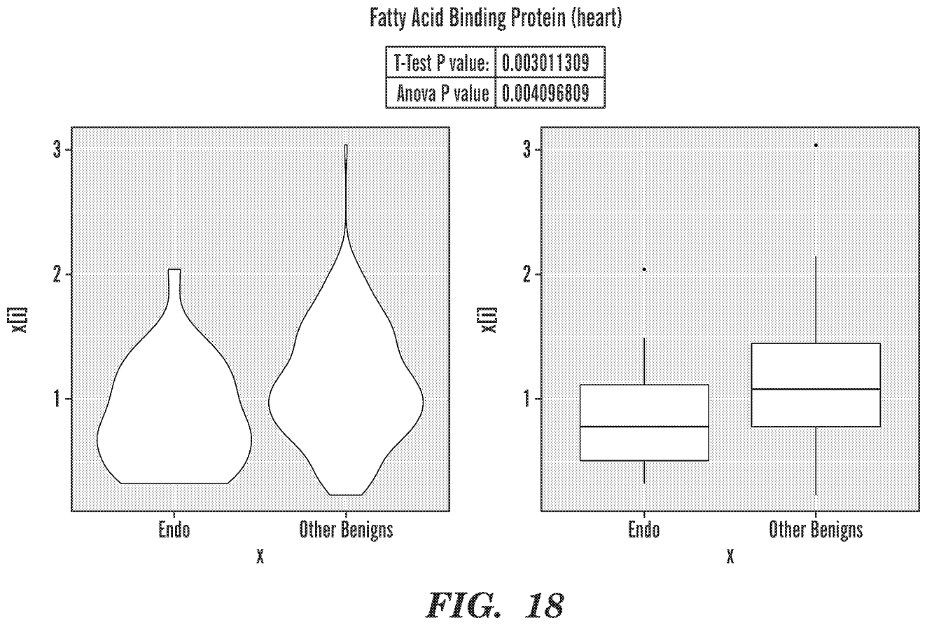

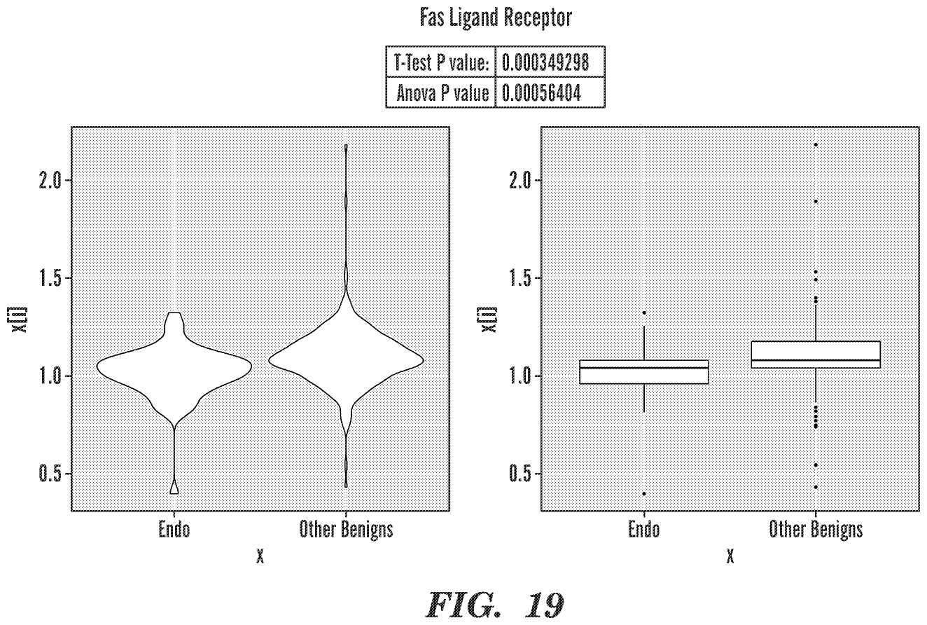

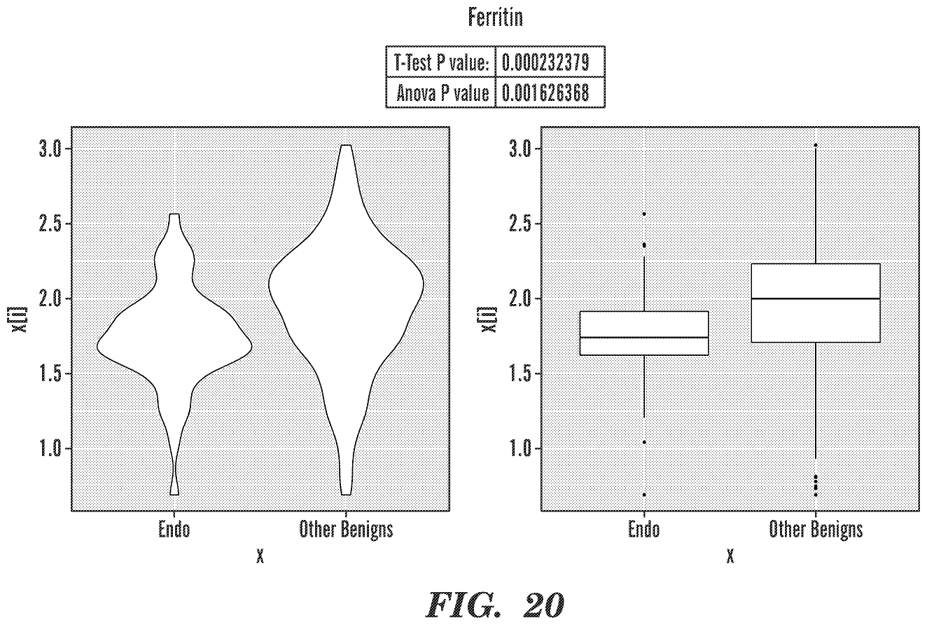

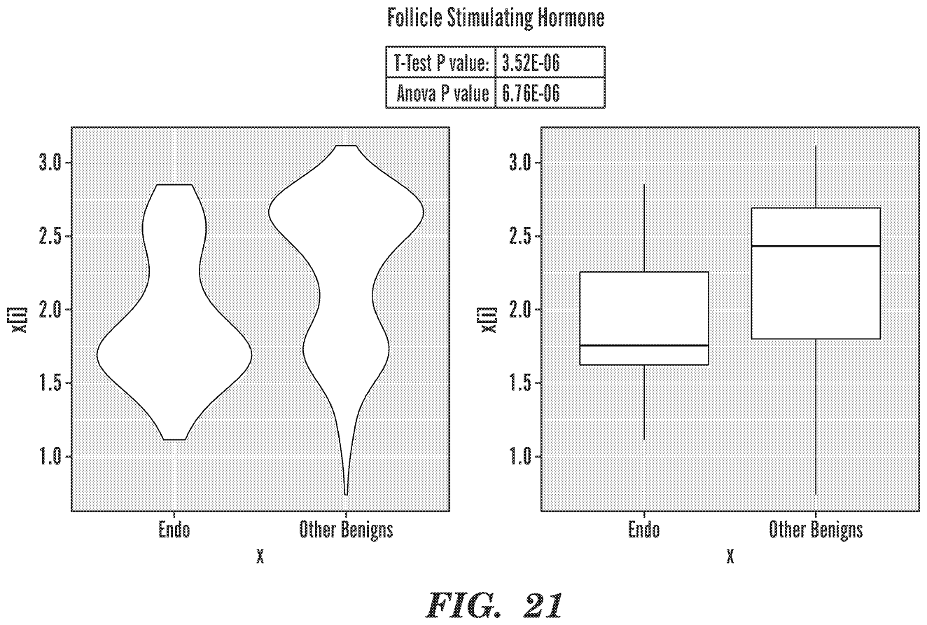

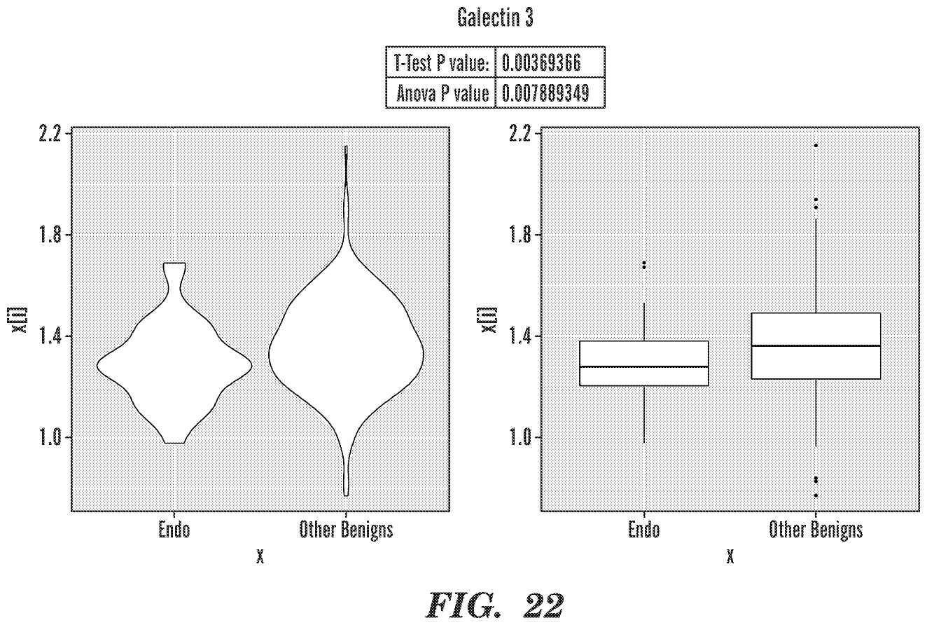

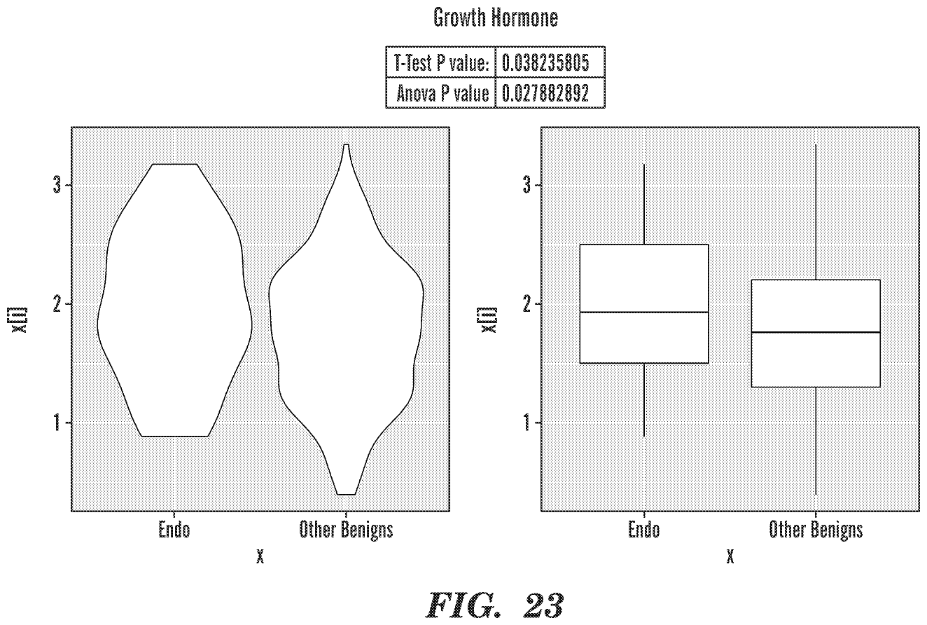

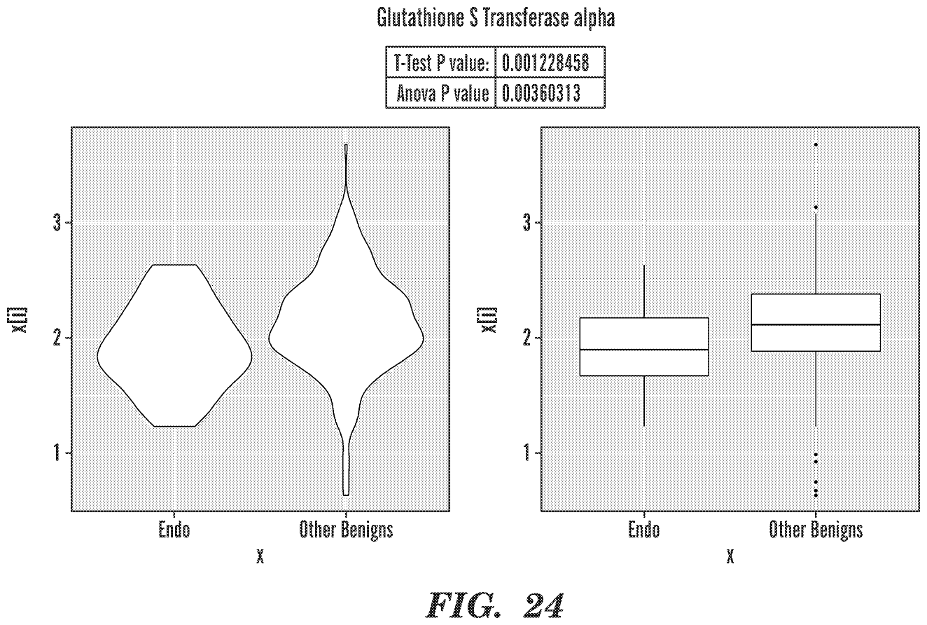

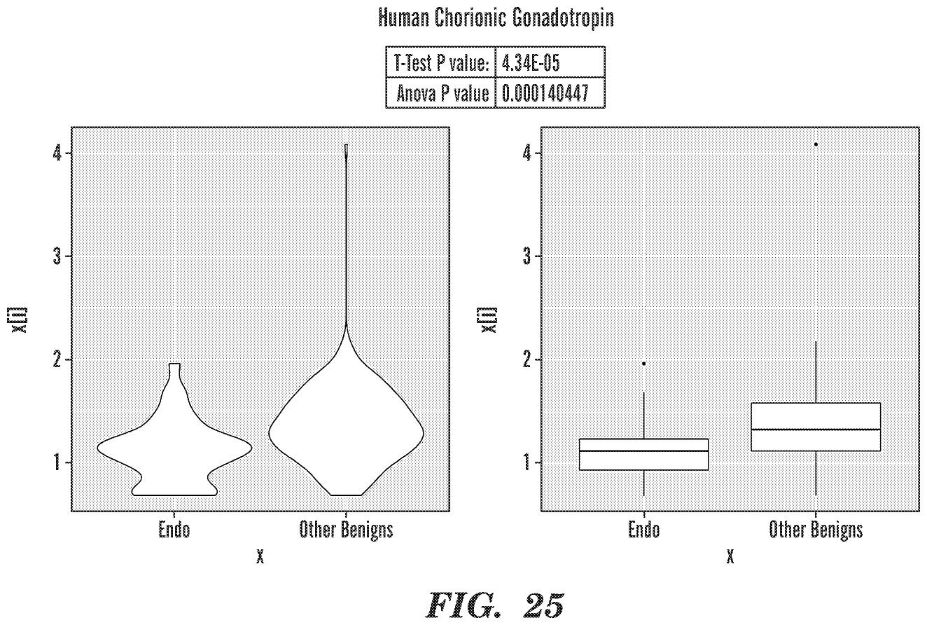

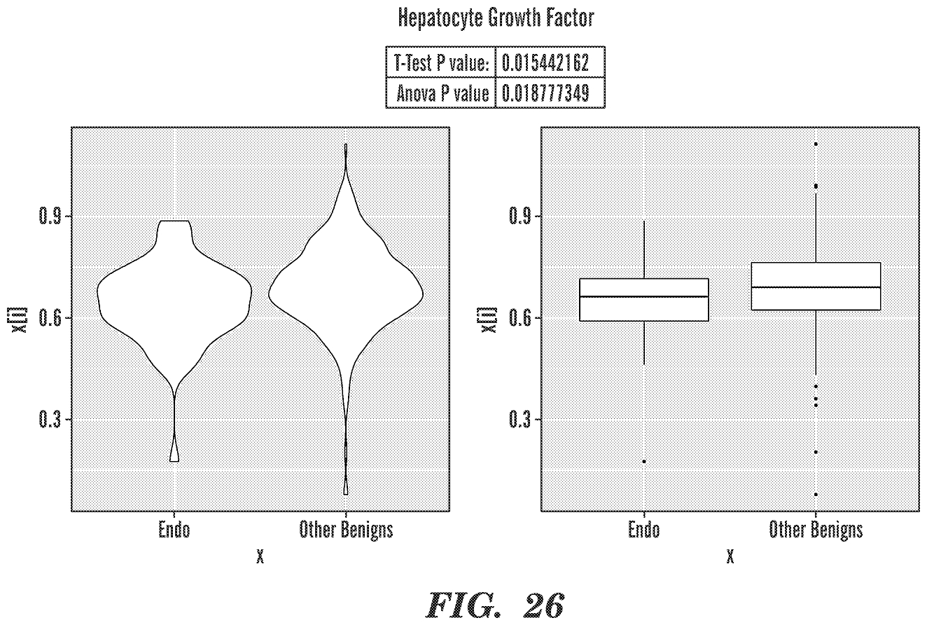

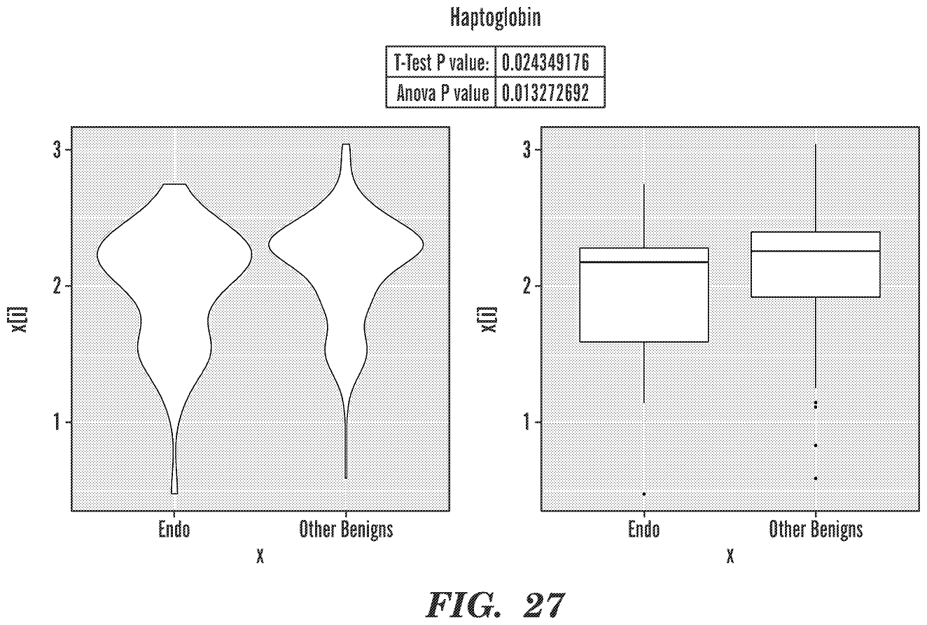

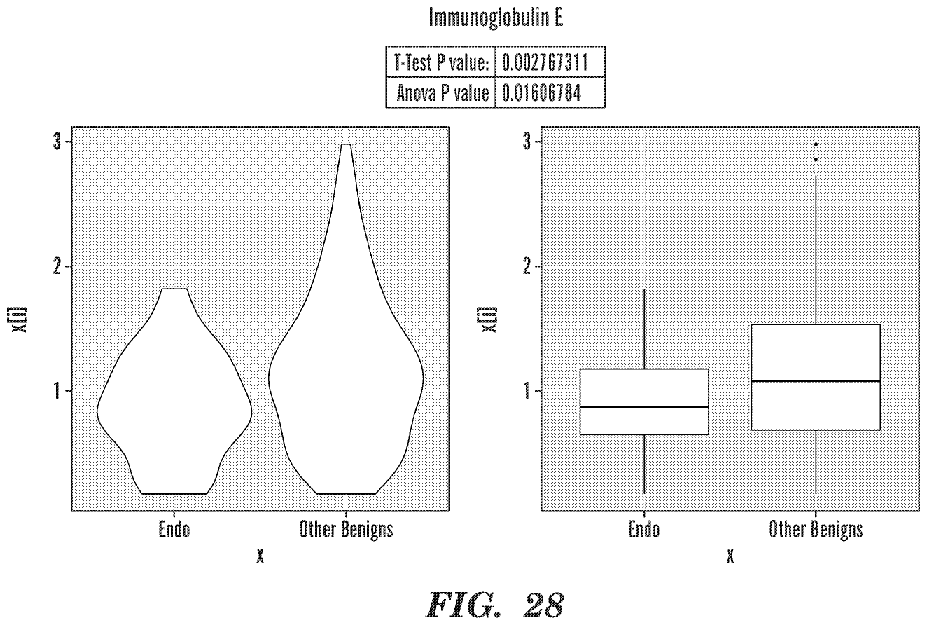

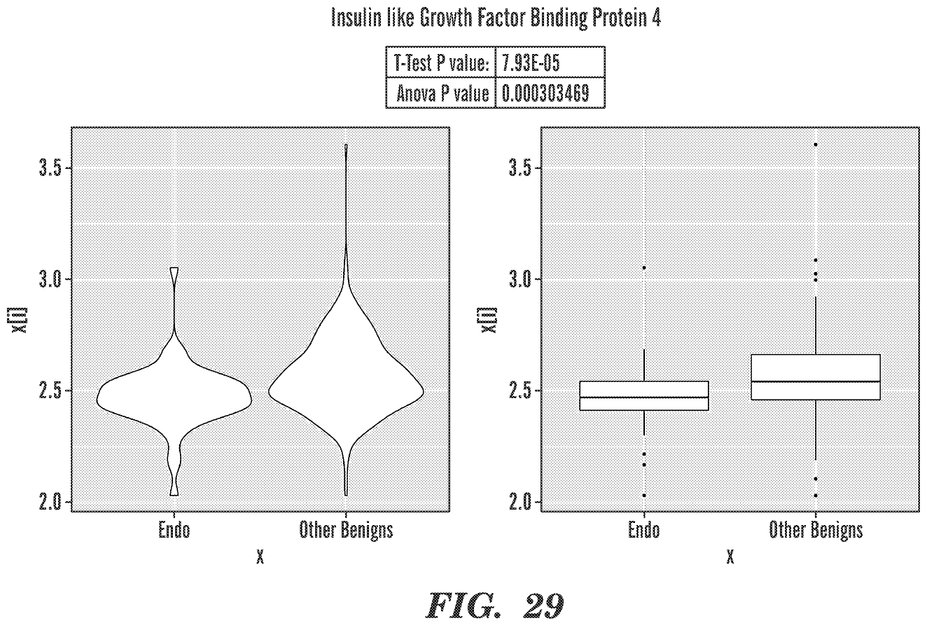

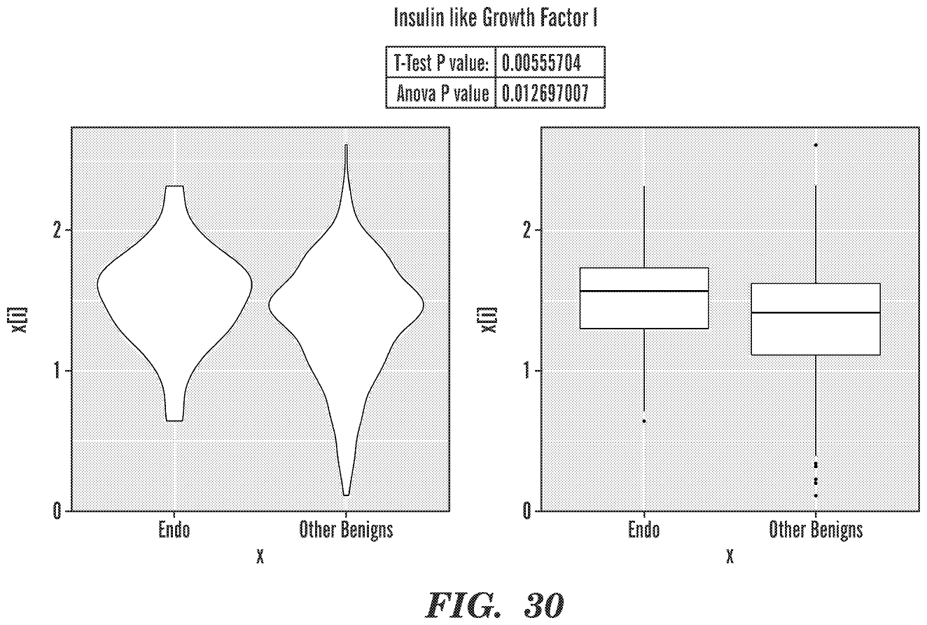

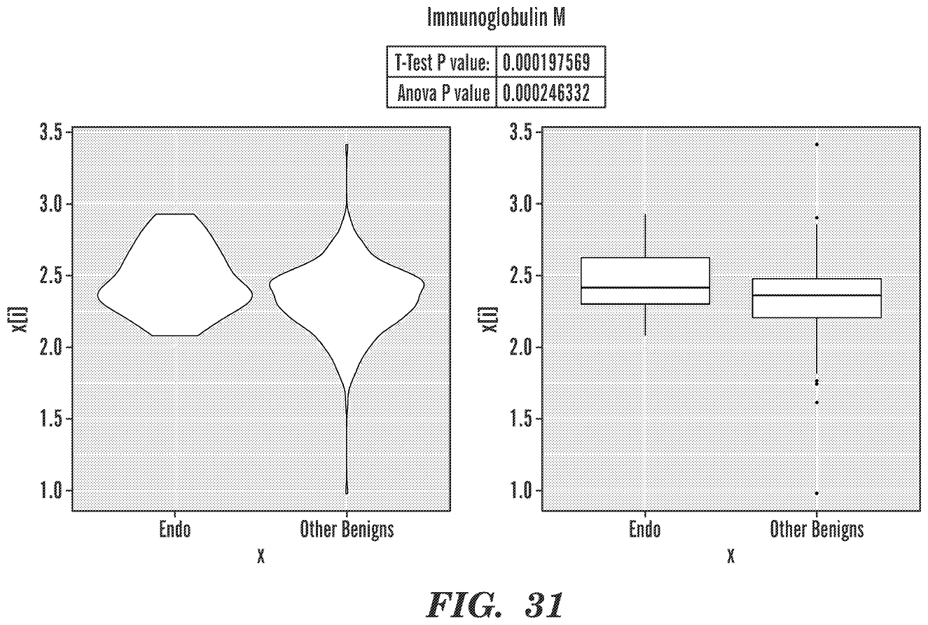

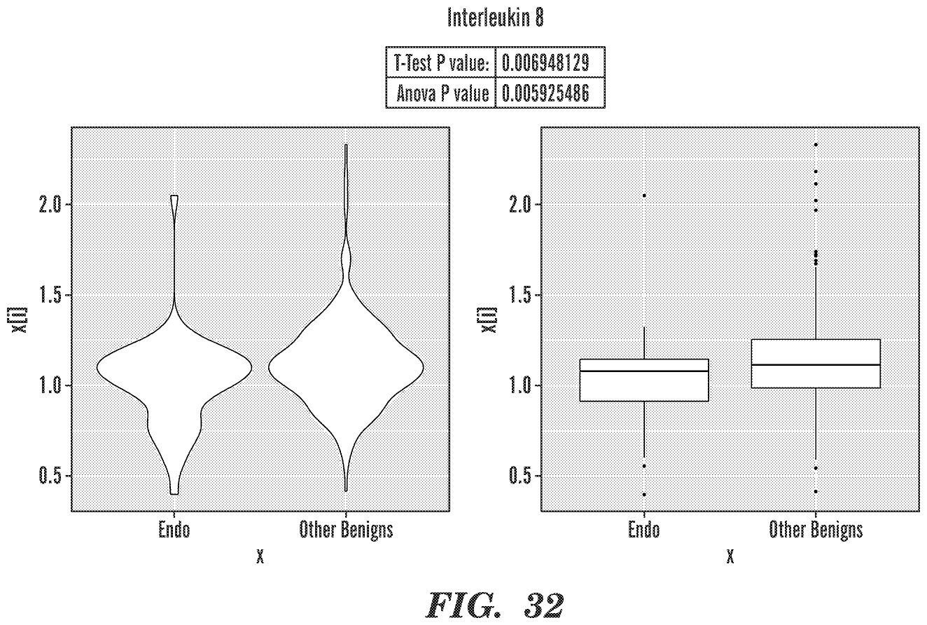

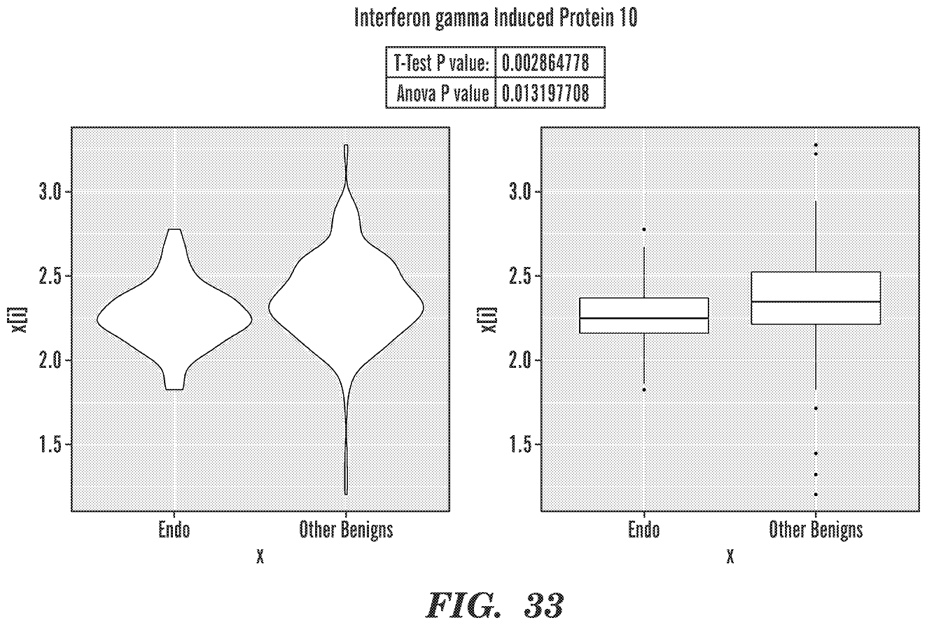

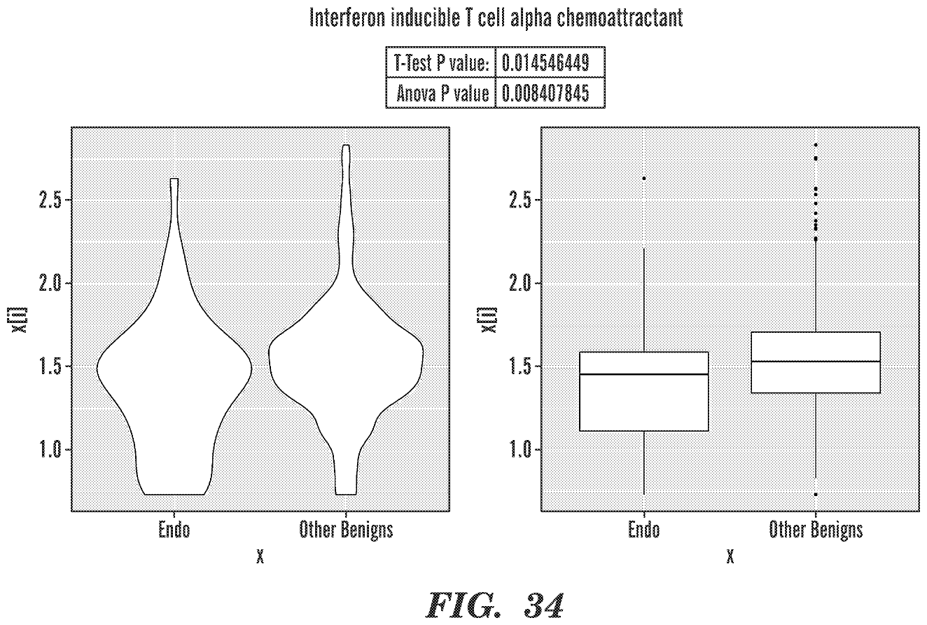

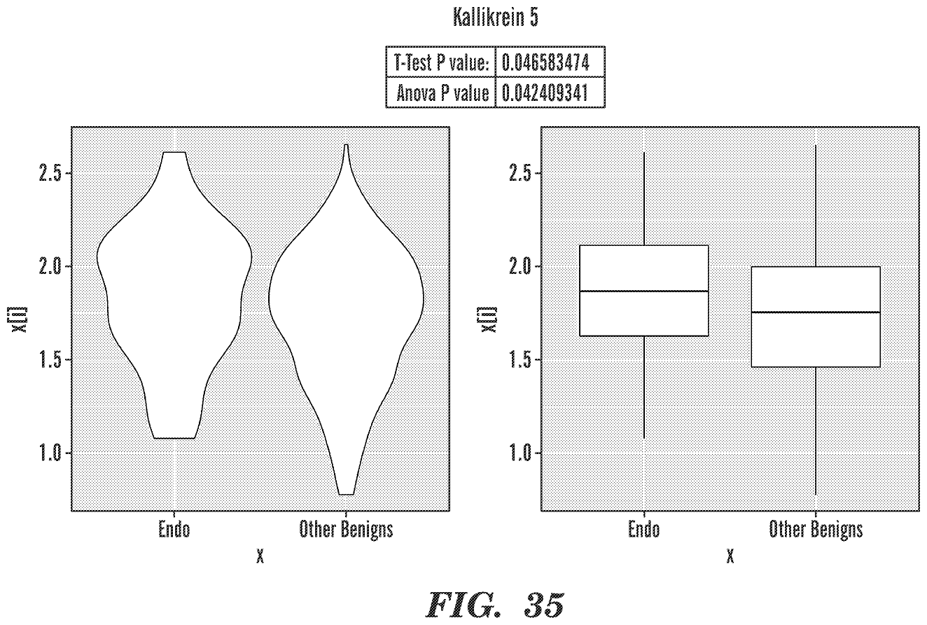

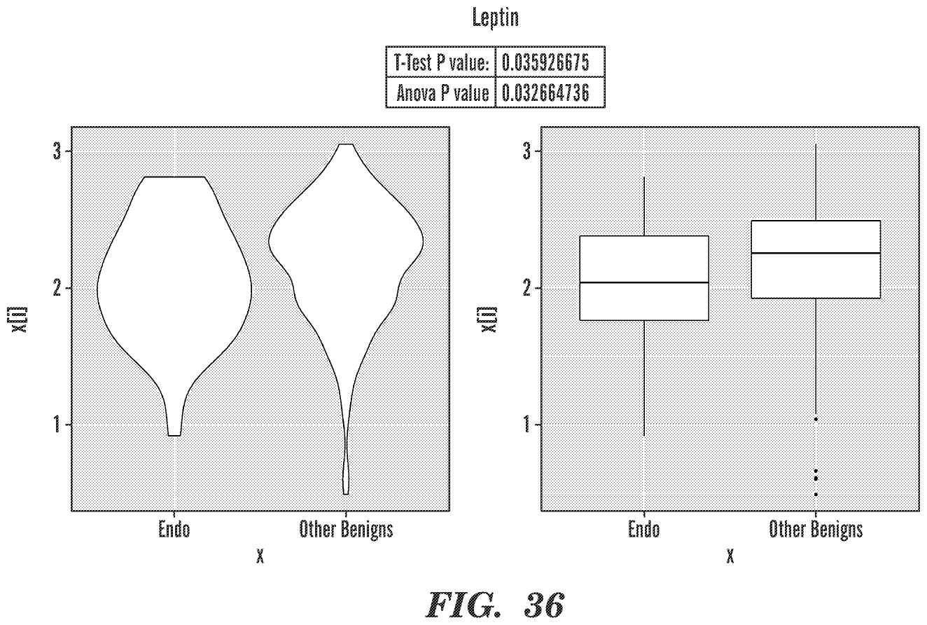

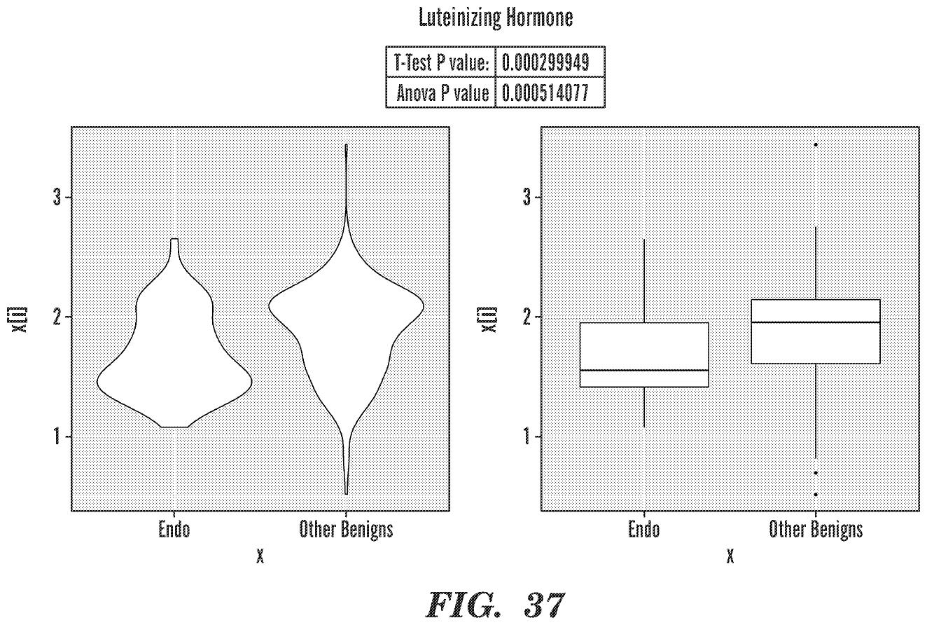

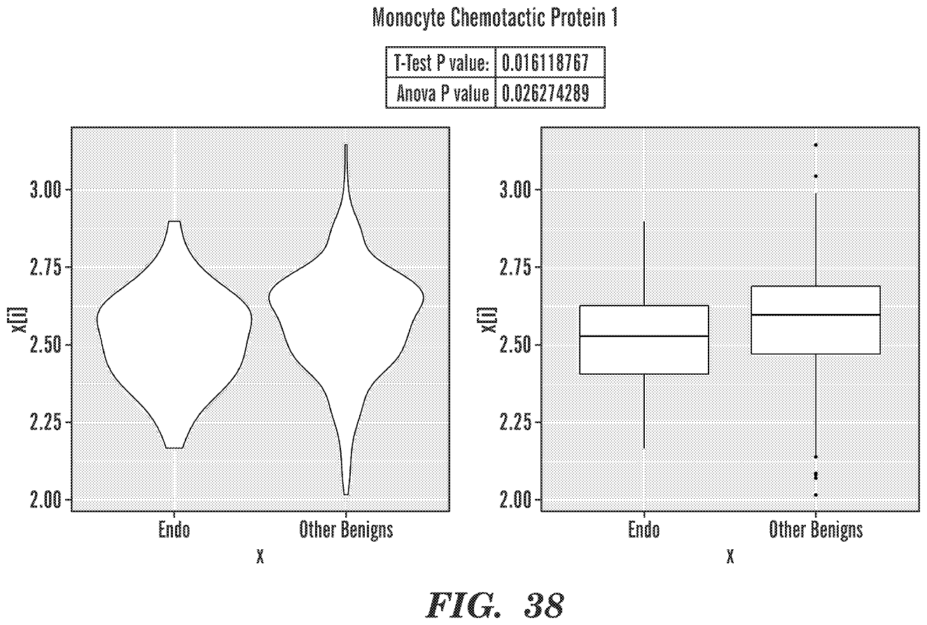

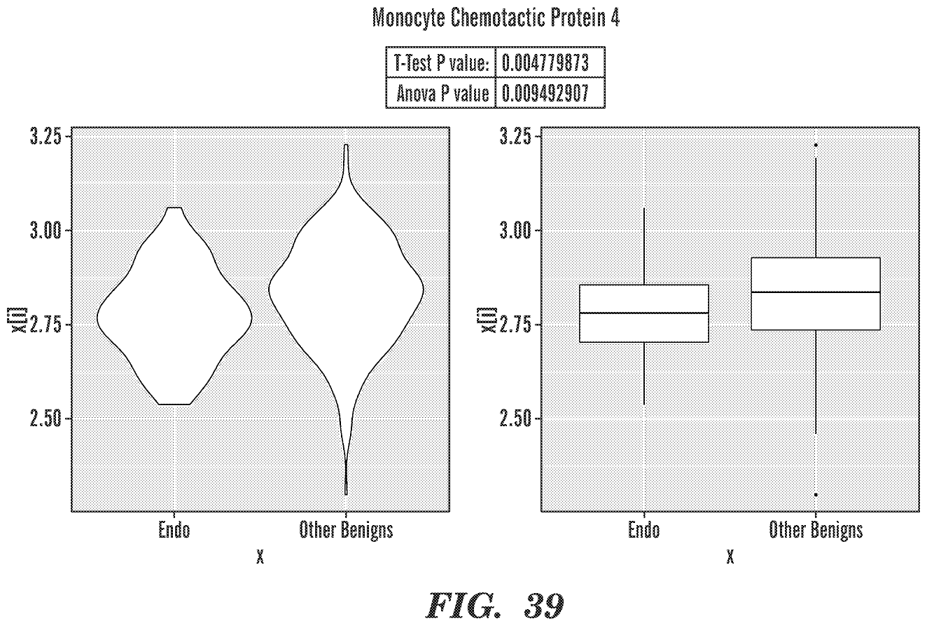

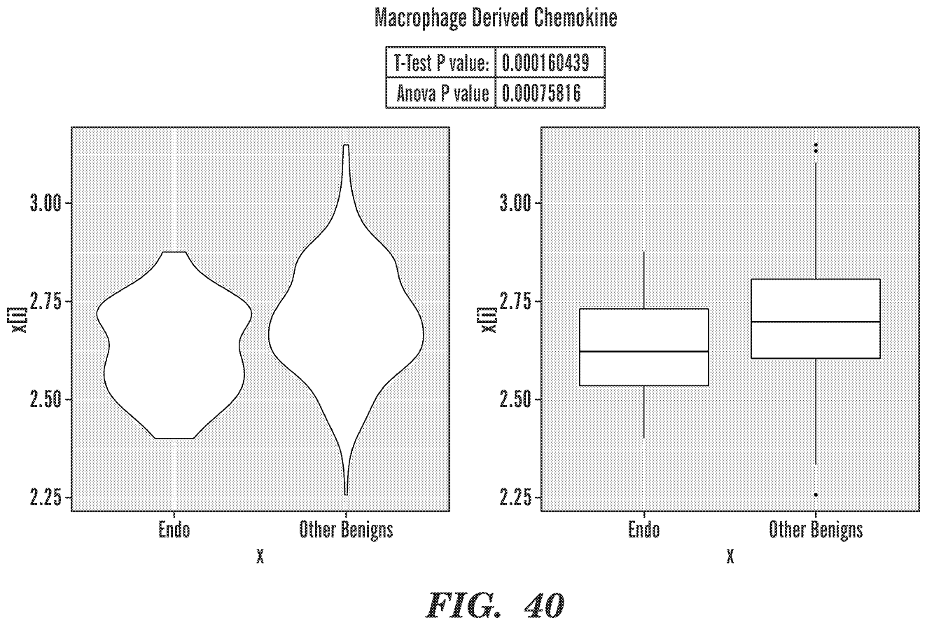

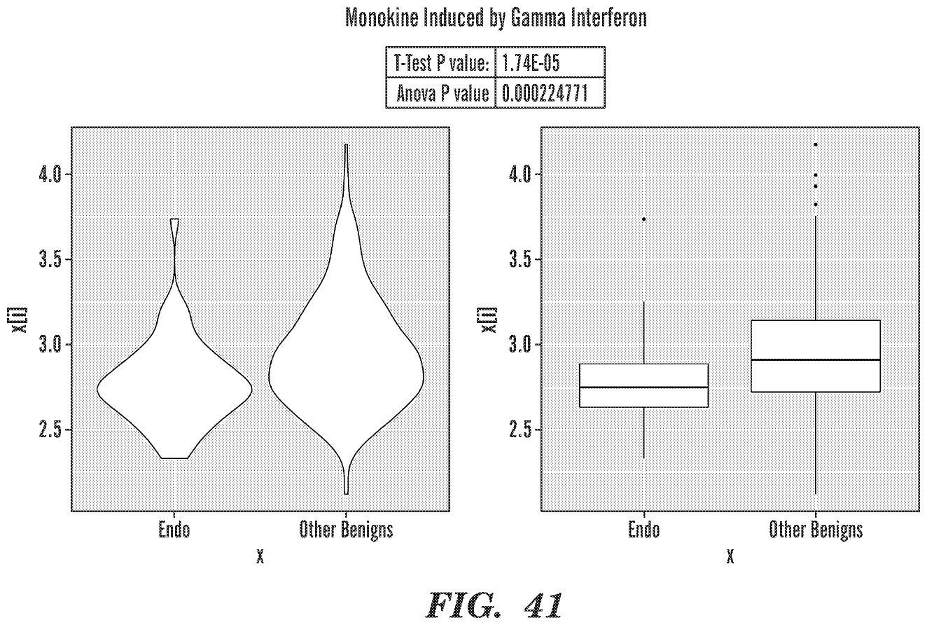

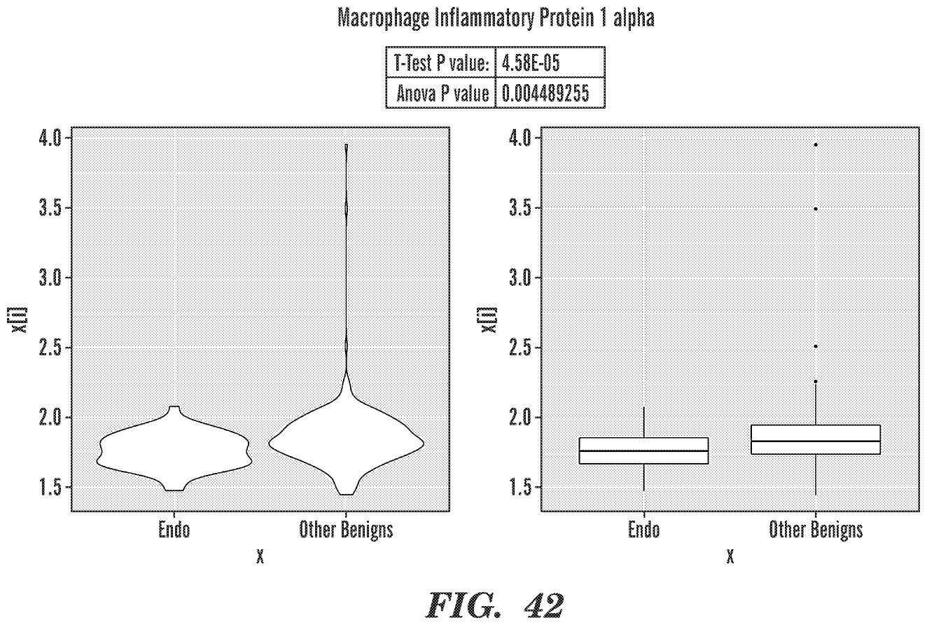

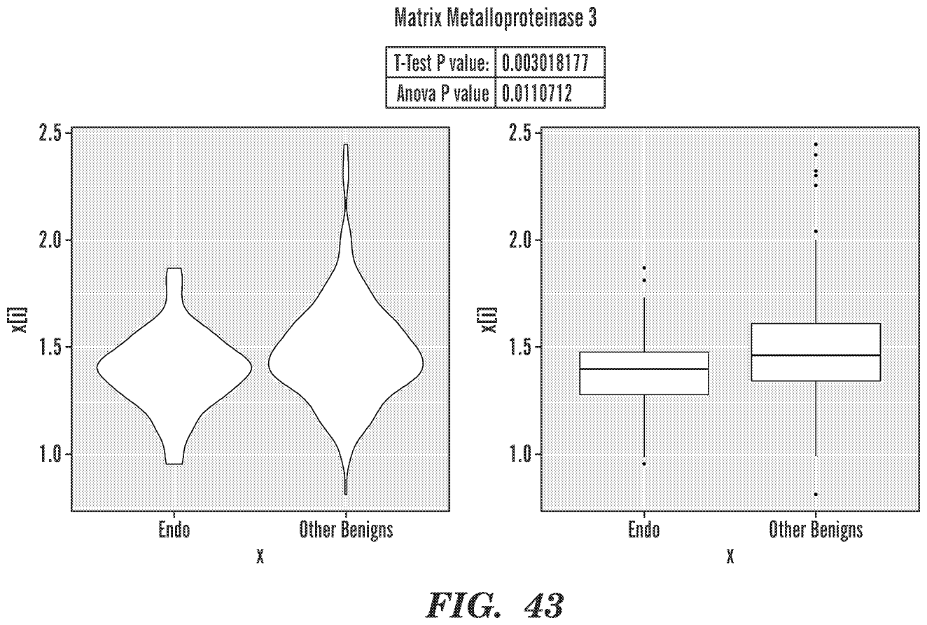

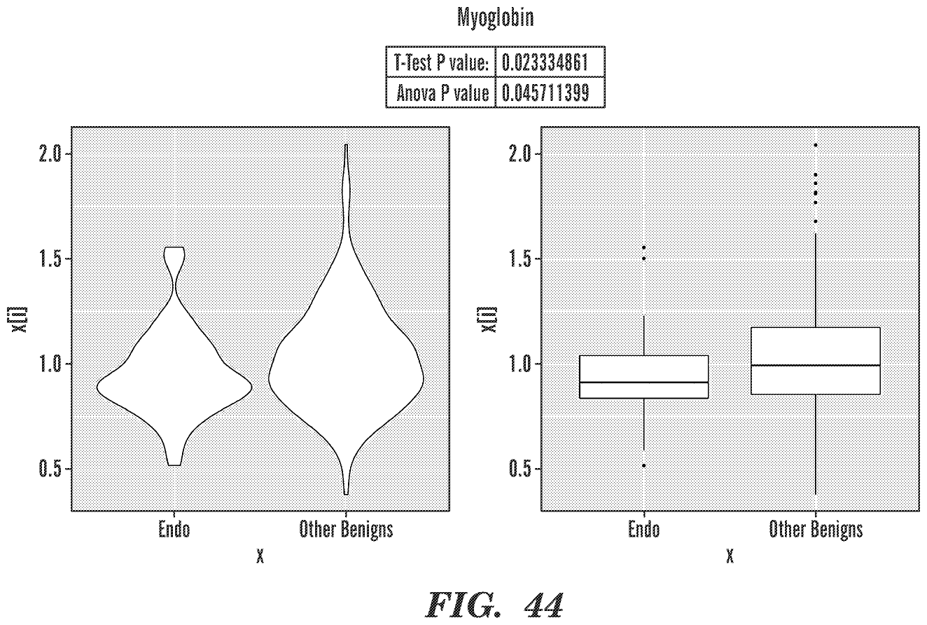

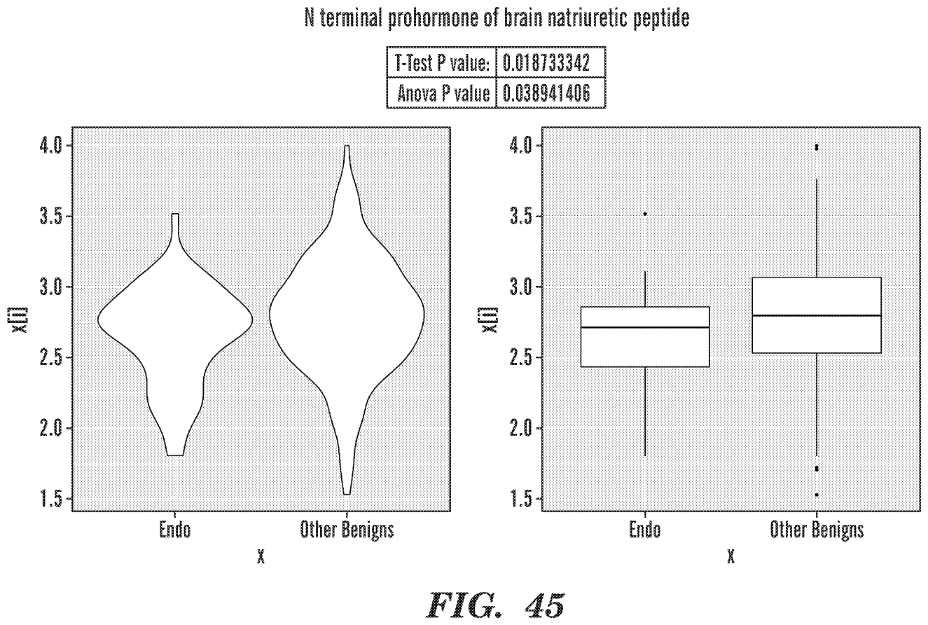

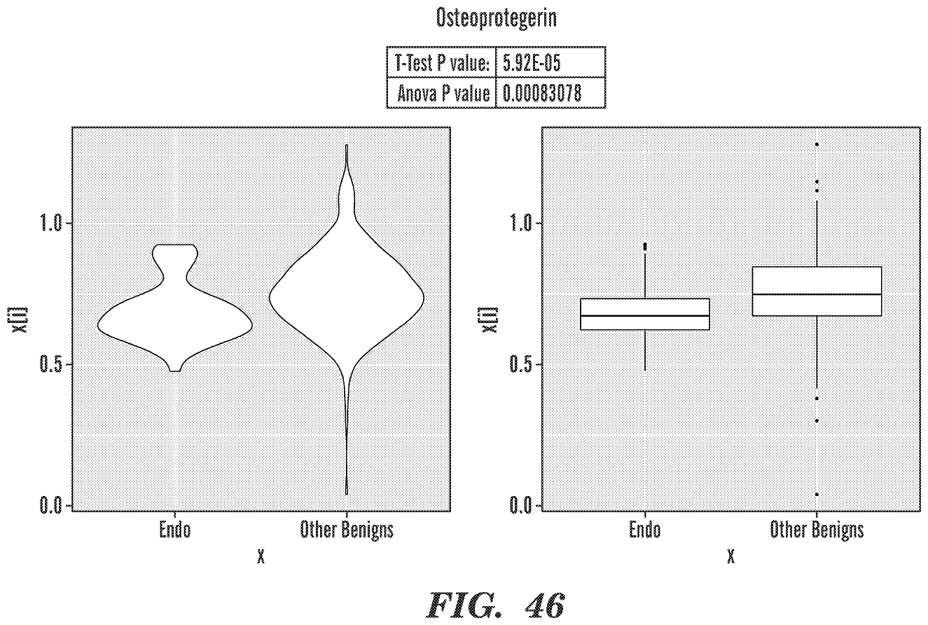

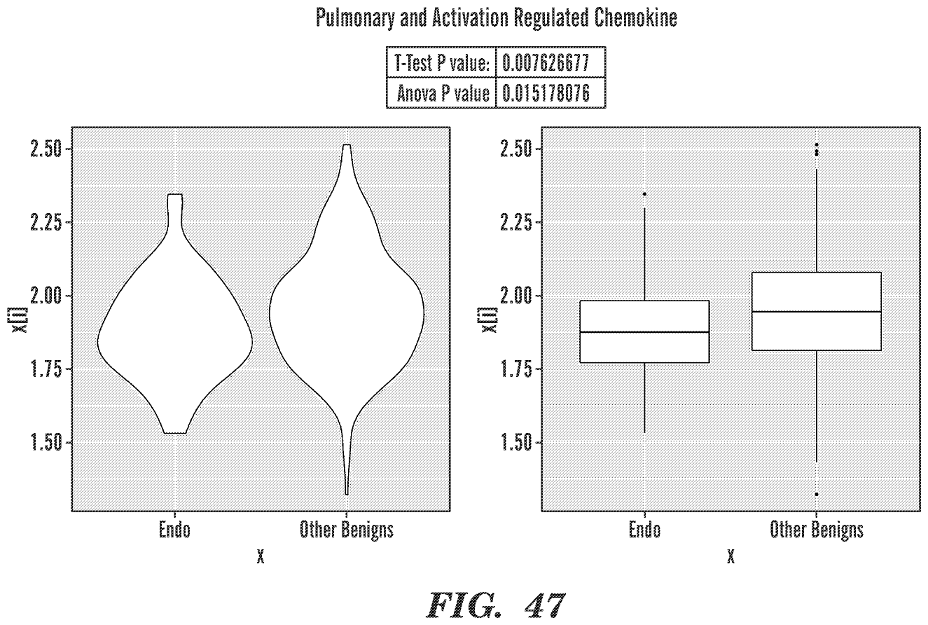

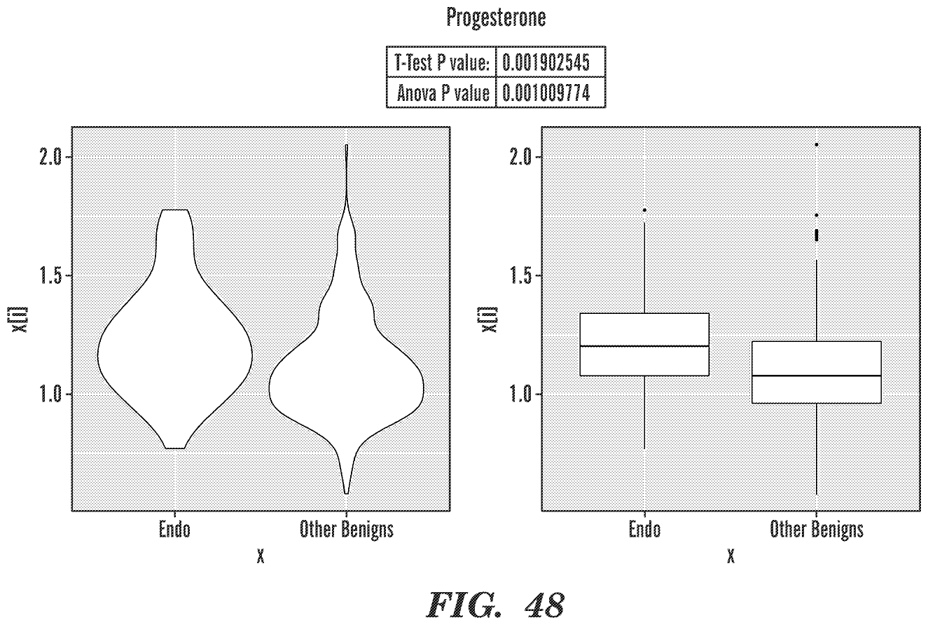

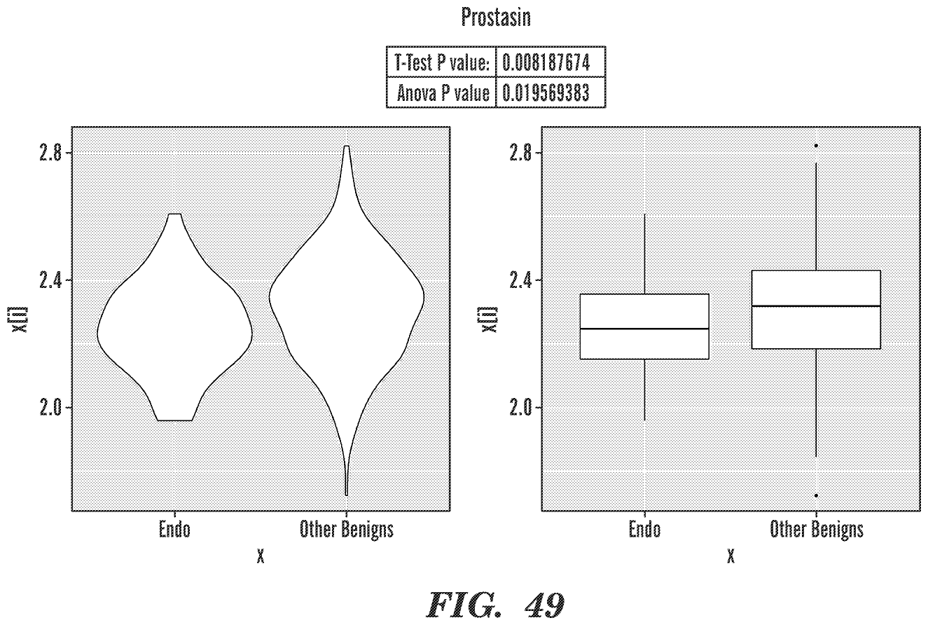

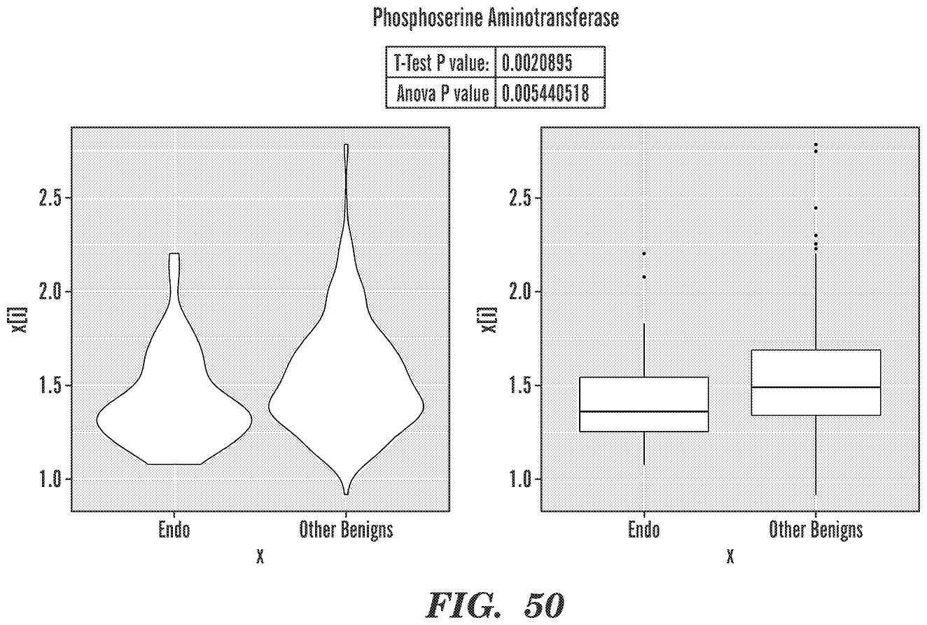

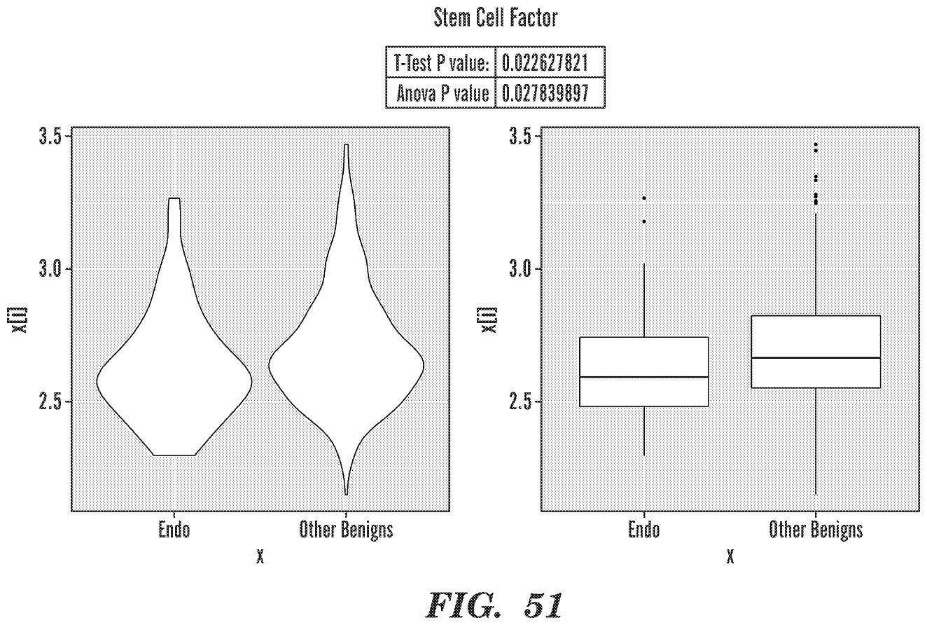

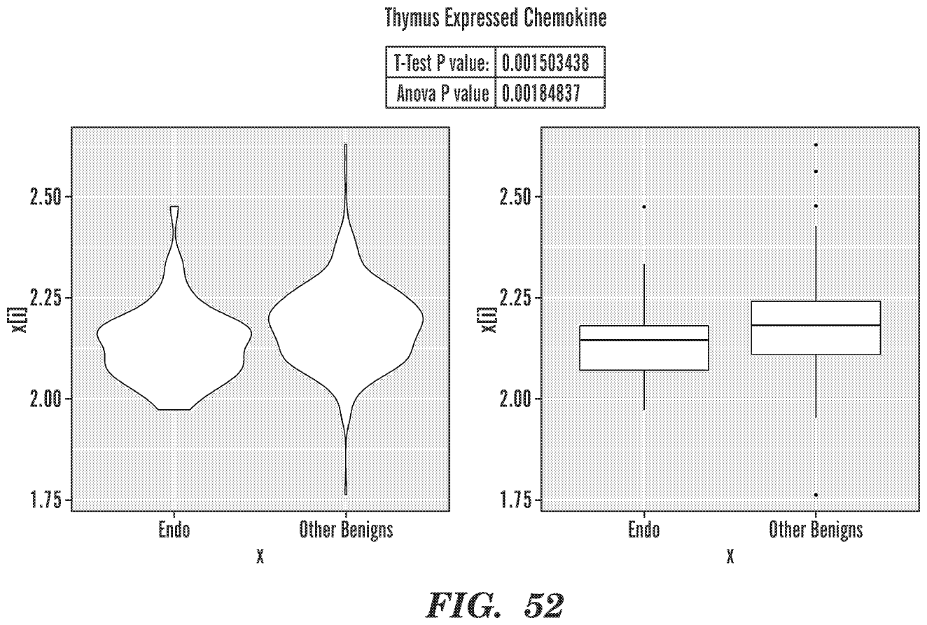

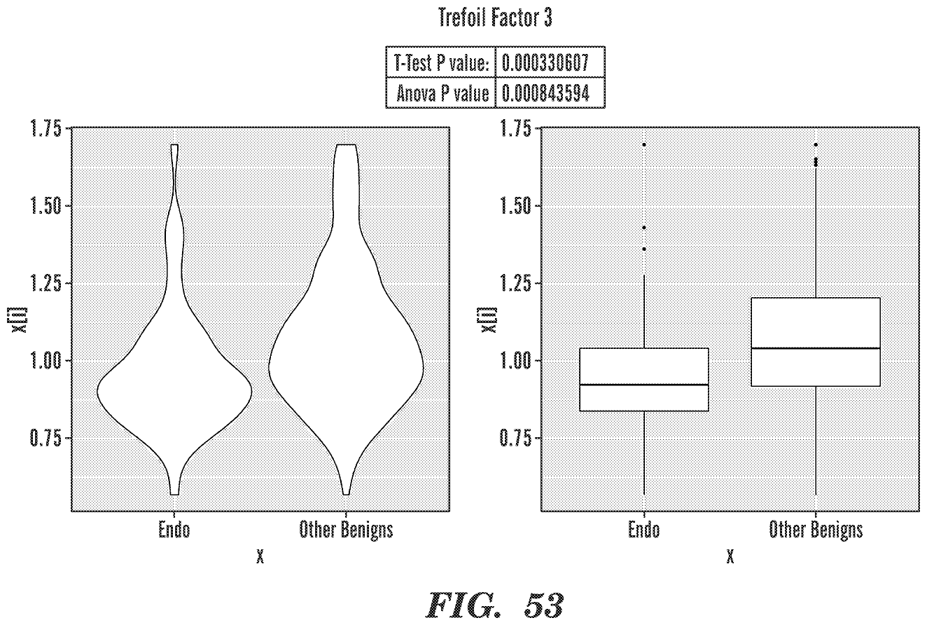

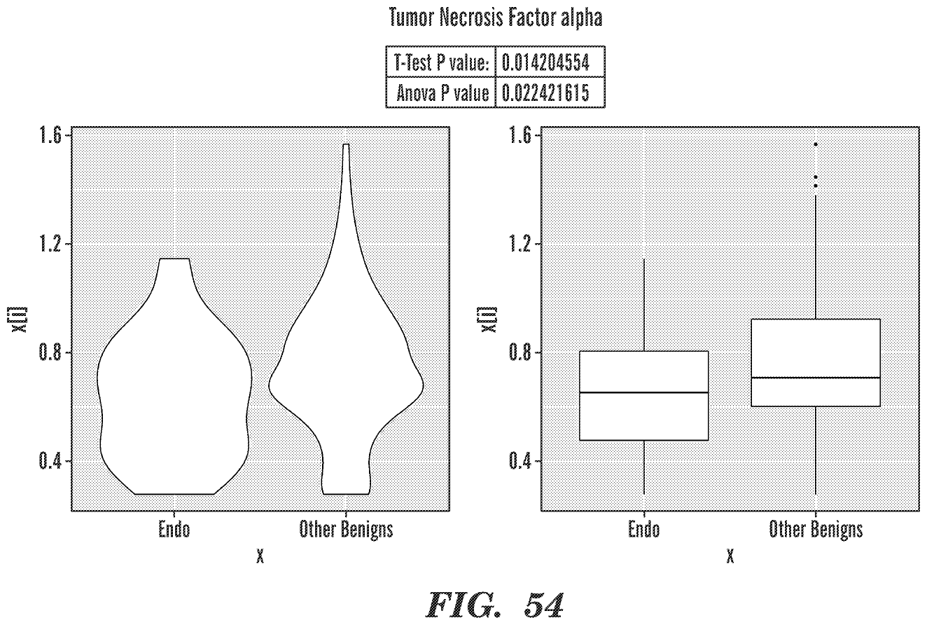

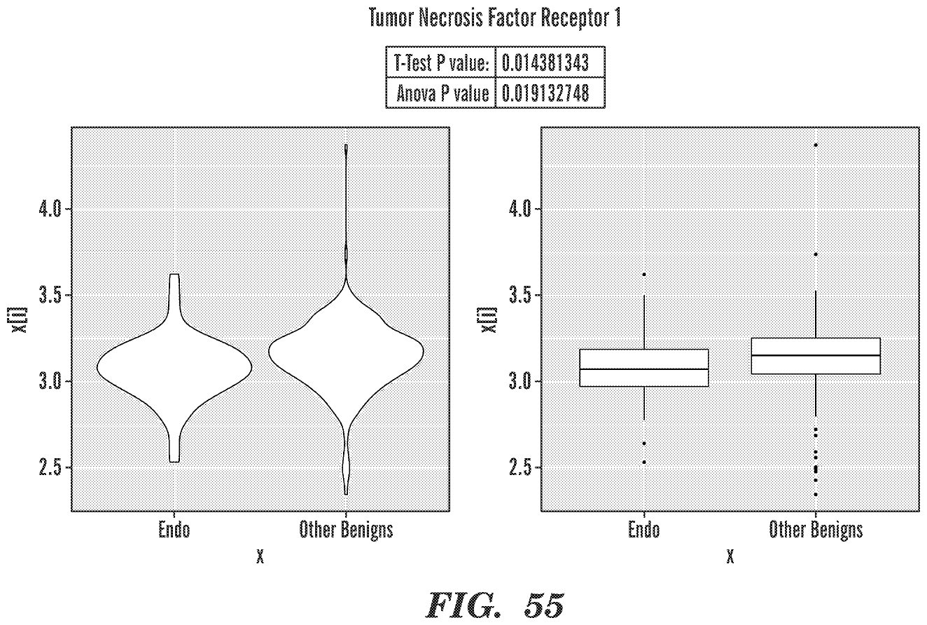

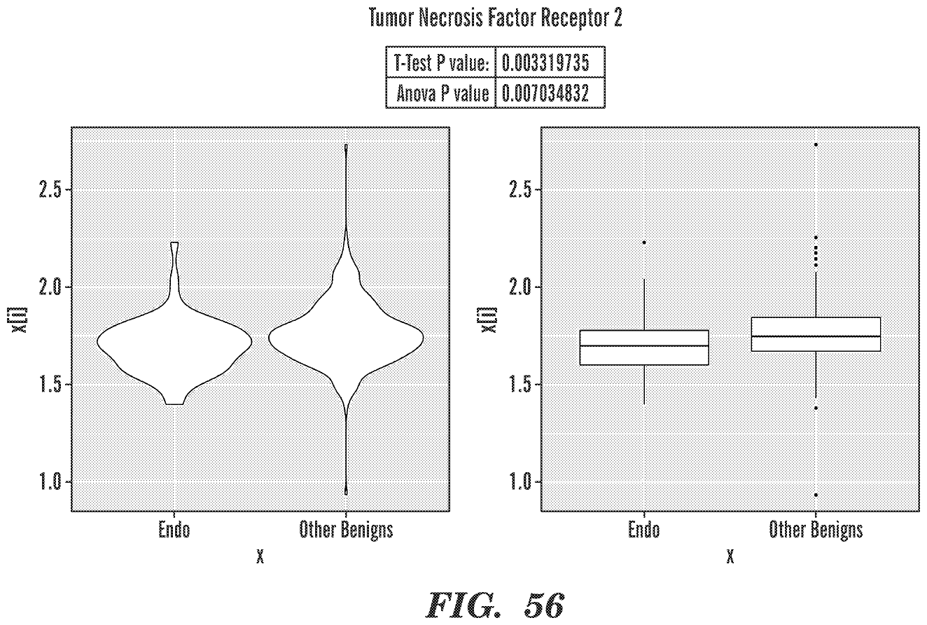

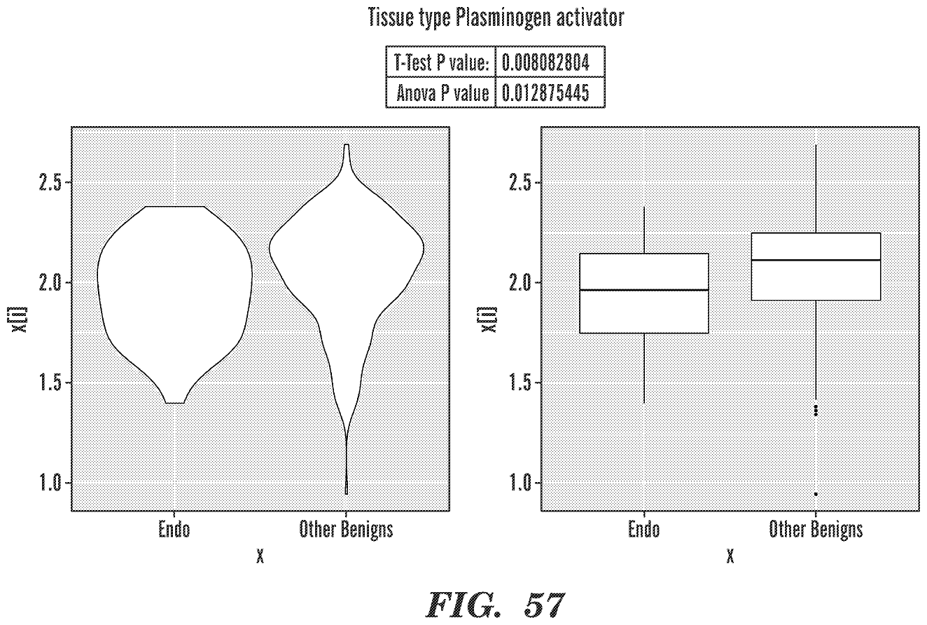

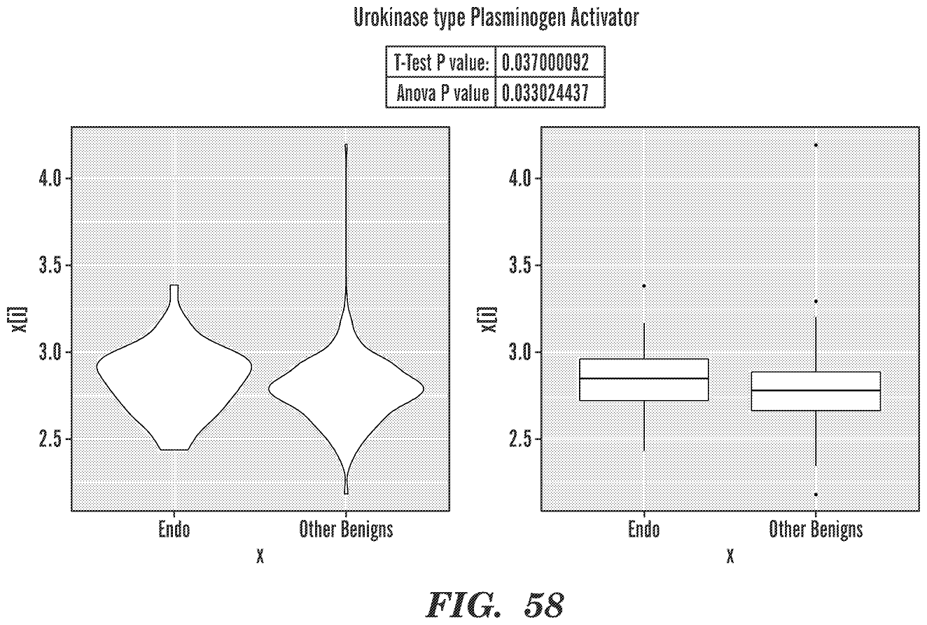

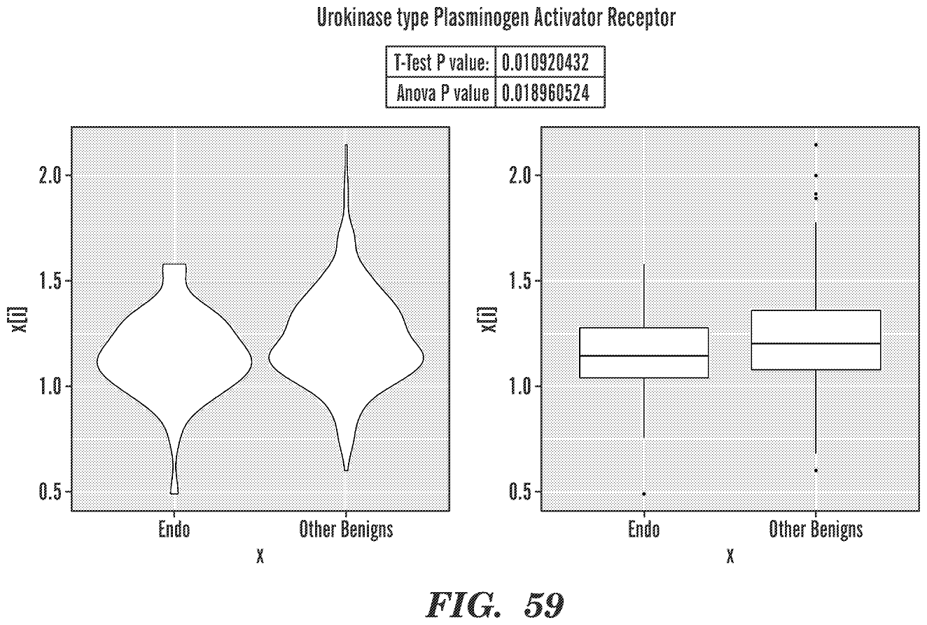

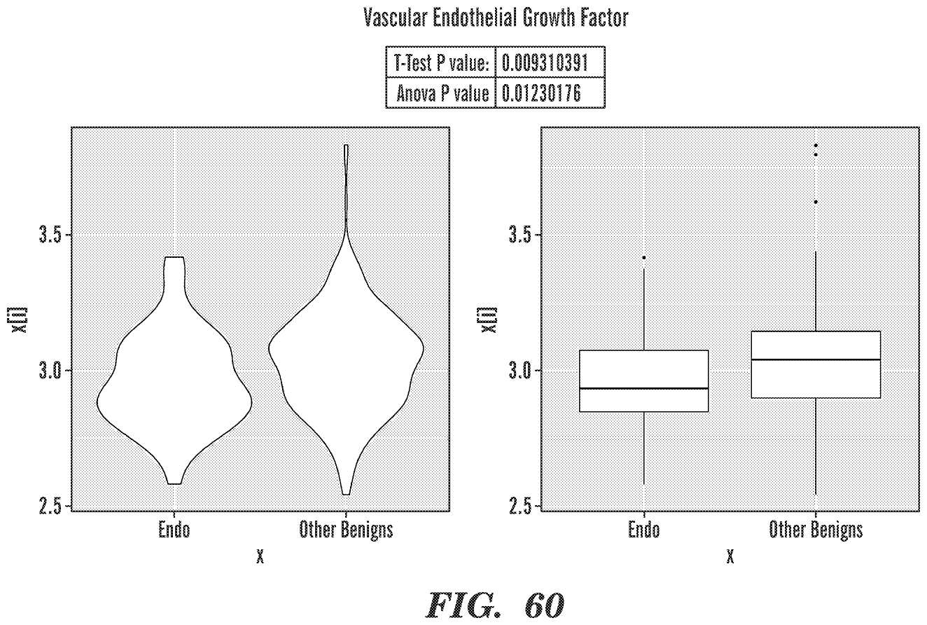

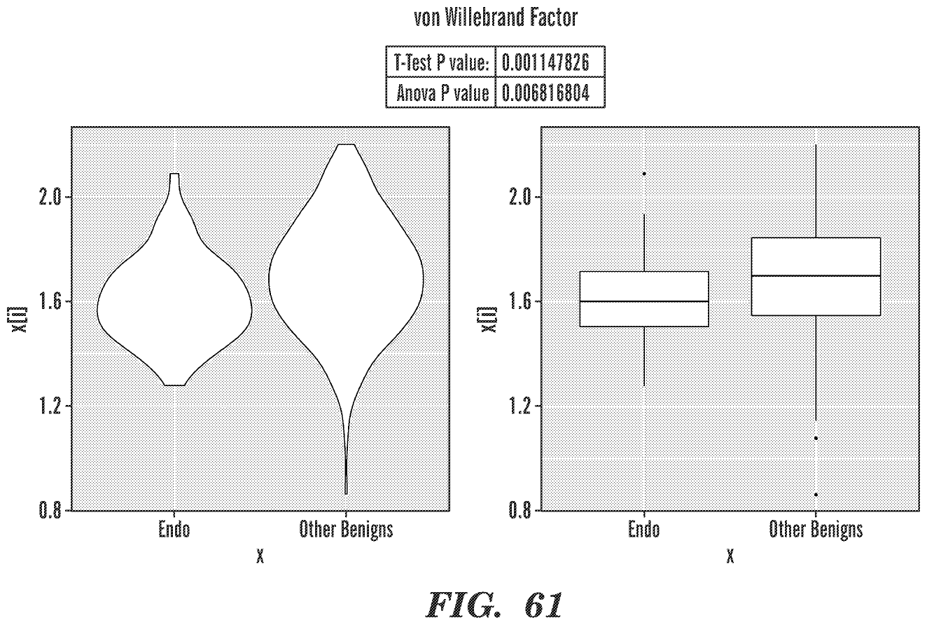

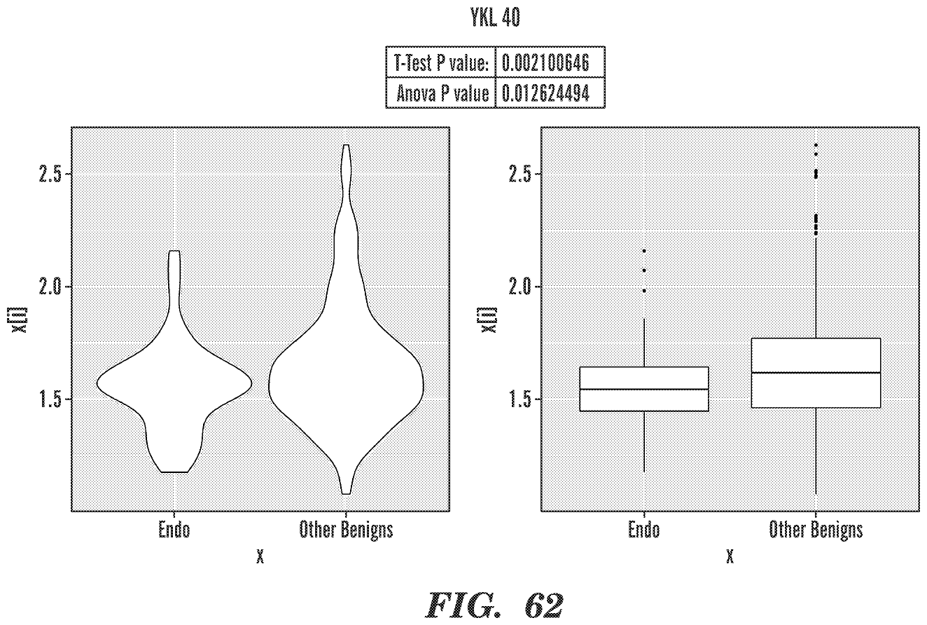

Unless stated otherwise, the control populations in studies cited here consisted of subjects with no known endometrial or gynecologic conditions. The results of the biomarker analysis are shown in FIGS. 2-62.

### Example 2: Dataset 1 Classifying Endometriosis Versus Non-Endometriosis Using Machine Learning

The dataset contained 511 subjects, 54 classified as endometriosis (EM code 1) and 457 classified as non-endometriosis (EM code 0). Of these 511 subjects, 224 were post-menopausal (code 1) and 287 were pre-menopausal (code 0). Of these 287 pre-menopausal subjects, 41 were endometriosis subjects and 246 were non-endometriosis subjects. The following nine features were used to build the model and in the analysis: ApoA1, B32M, CA125, TRF, TT/PREA, HE4, FSH, menopausal status, and age.

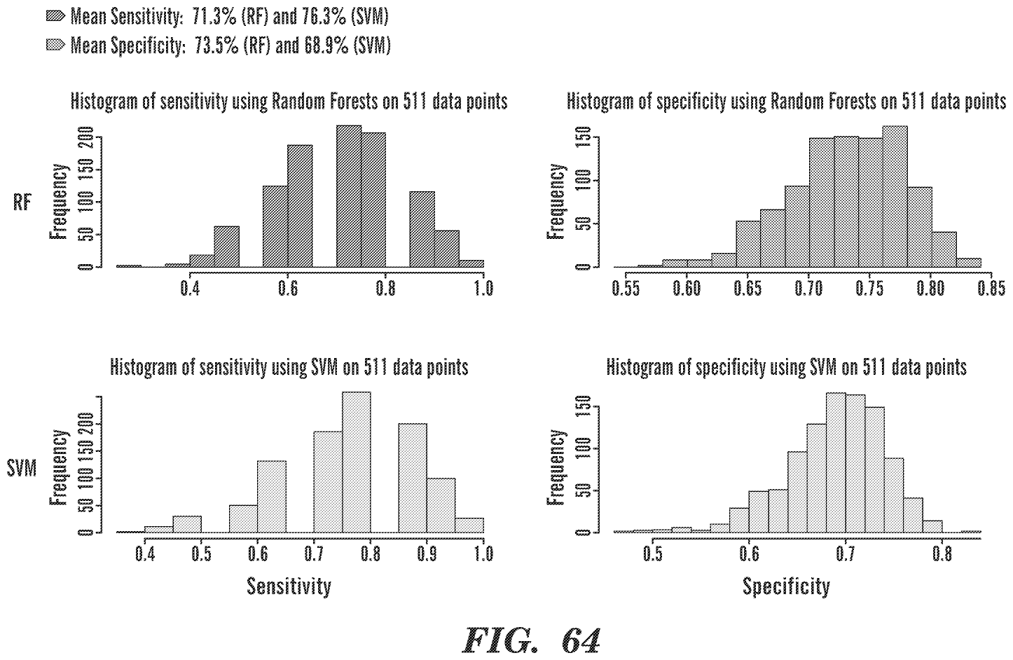

The classifier and performance reproducibility results are depicted in four histograms in FIG. 64, showing frequency versus sensitivity and specificity (left and right panels, respectively) using random forest (RF) and support vector machine (SVM) methods (top and bottom panels, respectively). The mean sensitivity was 71.3% for RF and 76.3% for SVM. The mean specificity was 73.5% for RF and 68.9% for SVM.

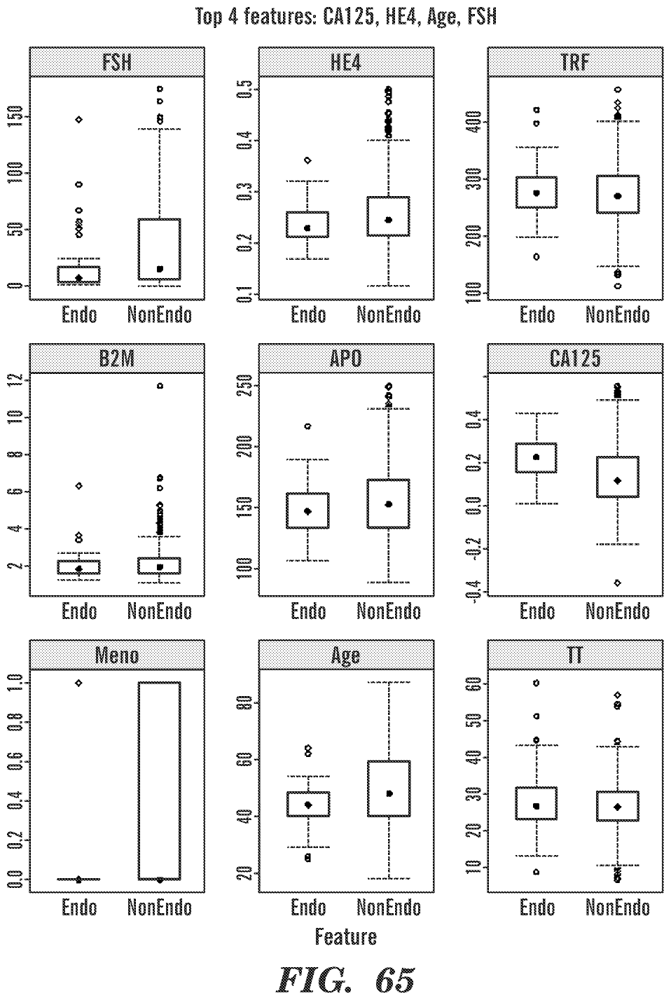

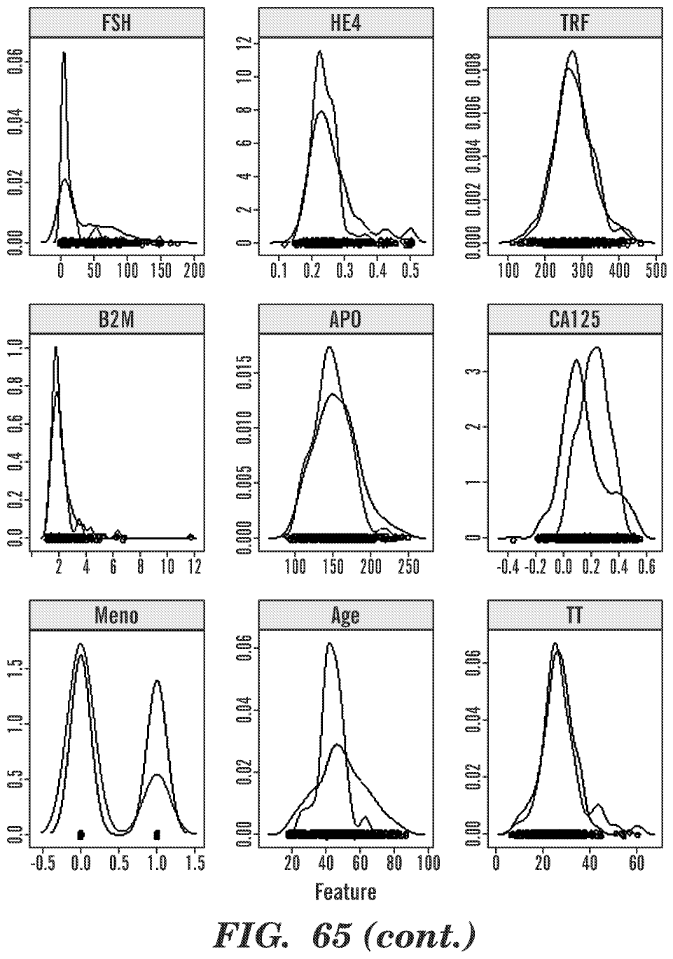

The features distribution results using the entire dataset are shown in FIG. 65, for FSH, HE4, TRF, B2M, ApoA1, CA125, TT, menopausal status, and age. The top four features were CA125, HE4, age, and FSH.

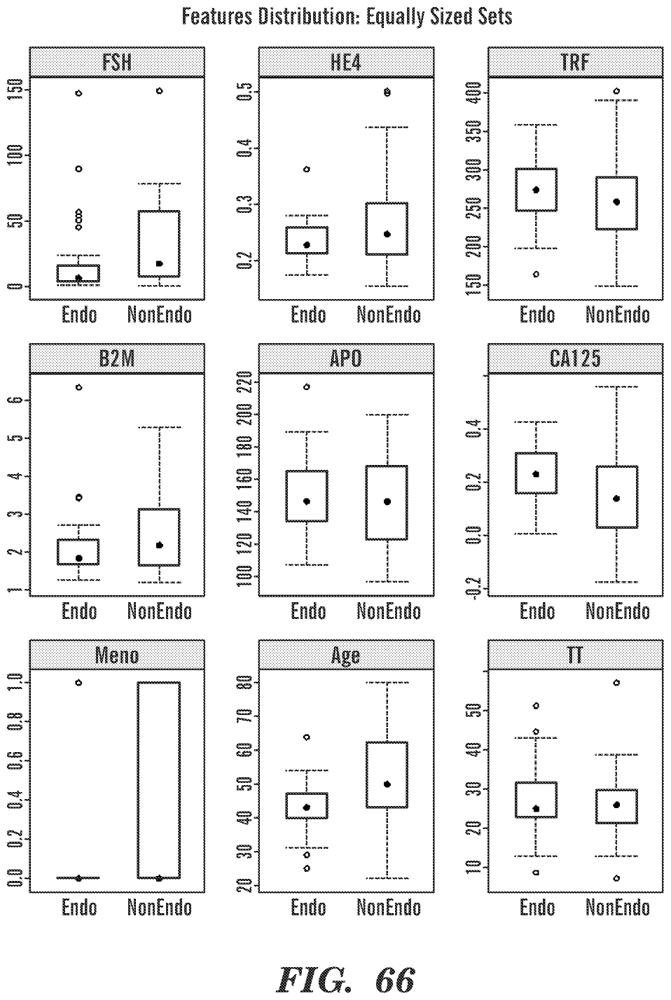

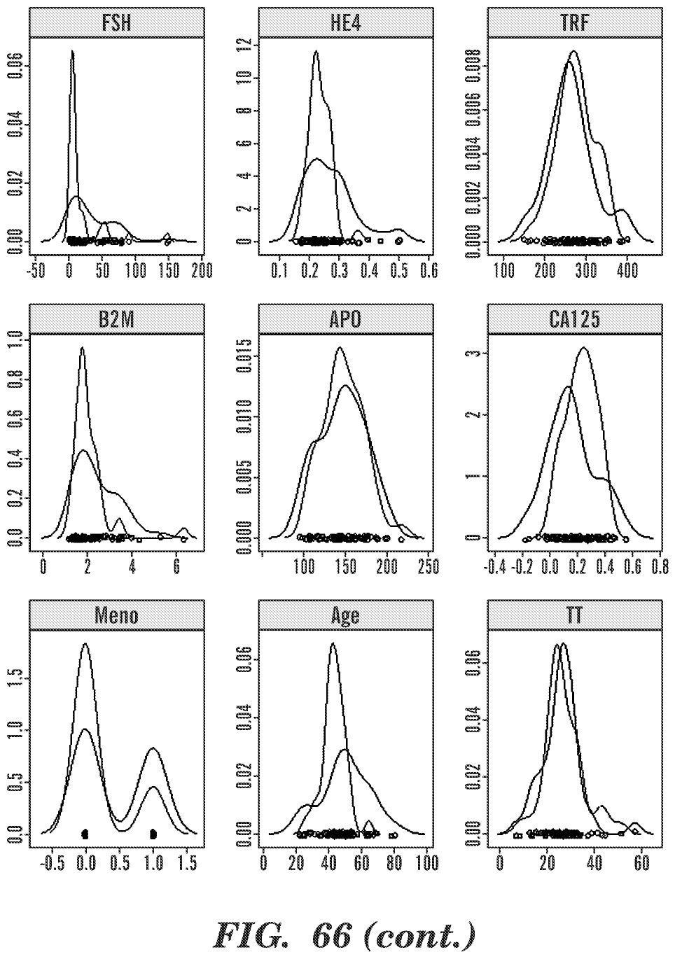

The features distribution results using equally sized datasets are shown in FIG. 66, for FSH, HE4, TRF, B2M, ApoA1, CA125, TT, menopausal status, and age.

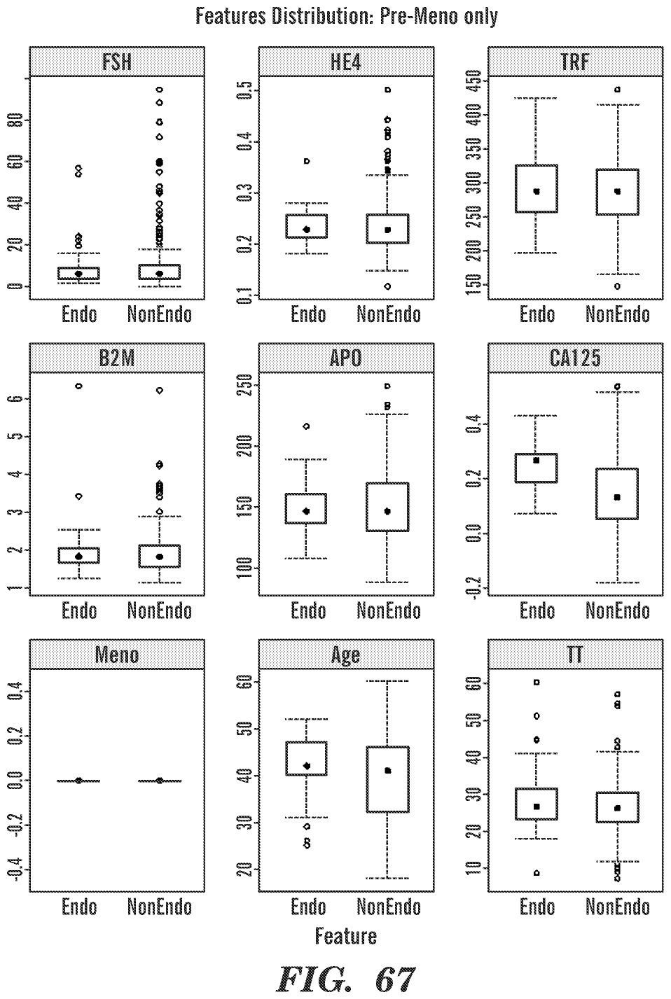

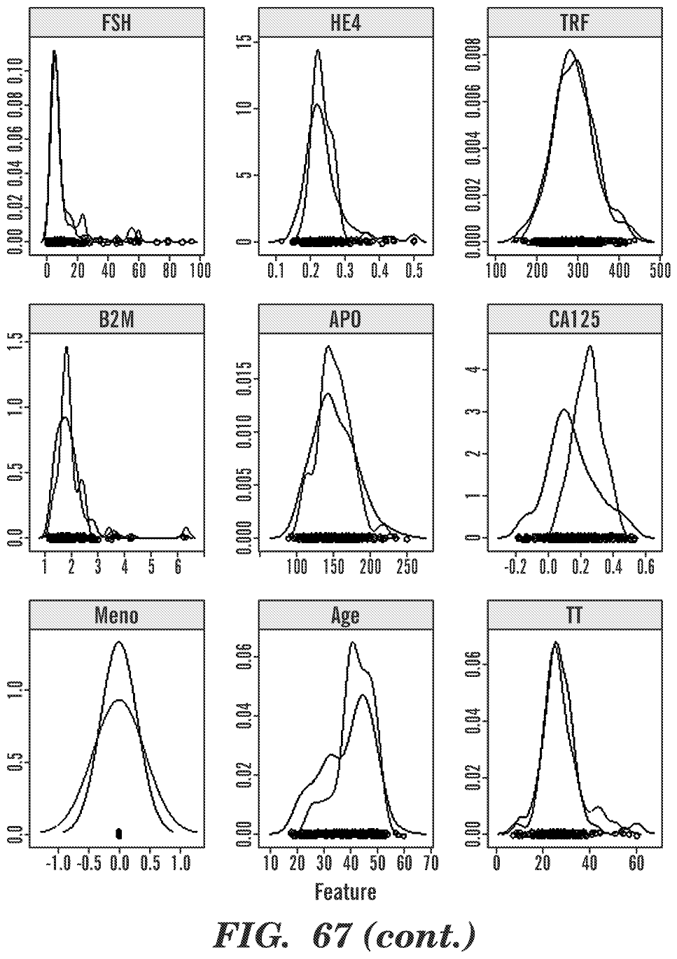

The features distribution results using pre-menopausal status only are shown in FIG. 67, for FSH, HE4, TRF, B2M, ApoA1, CA125, TT, menopausal status, and age.

### Example 3: Dataset 2 Classifying Endometriosis Versus Non-Endometriosis Using Machine Learning

Two datasets, Bristow and 522 (described in Example 5) were combined. The 522 dataset contained 1159 benign subjects, with no endometriosis (Code 1), plus 92 cancer subjects (Code 2), plus 156 benign subjects, with endometriosis (code 3). The Bristow dataset contained 357 benign subjects, with no endometriosis, plus 96 cancer subjects, plus 58 benign subjects, with endometriosis.

The combined dataset thus contained 1516 benign subjects, with no endometriosis, plus 188 cancer subjects, plus 214 benign subjects, with endometriosis. Subjects that were post-menopausal, or where menopausal status was unknown, were removed, resulting in the final dataset which contained 186 endometriosis subjects plus 931 non-endometriosis subjects. The following features were used to build the model: ApoA1, B2M CA125, TRE, TT/PREA, HE4 FSH menopausal status, age OVA1, and OVERA.

Scale transformation was employed, dividing by the standard deviation, thus creating a training set of 150 endometriosis subjects and 150 non-endometriosis subjects. 100 iterations using random forest (RF) and support vector machine (SVM) were run.

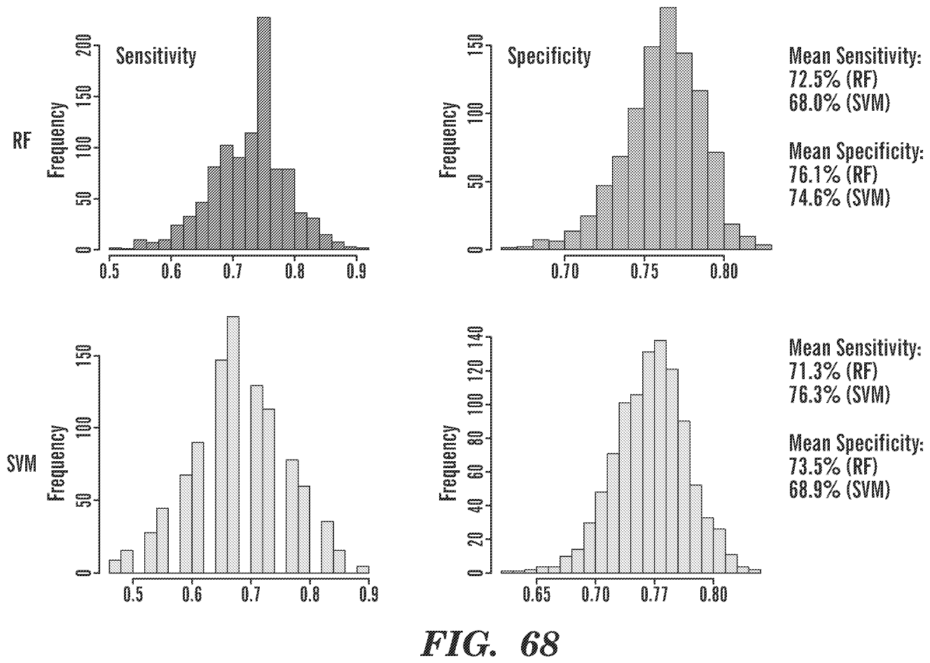

The classifier and performance reproducibility results are depicted in four histograms in FIG. 68, showing frequency versus sensitivity and specificity (left and right panels, respectively) using RE and SVM methods (top and bottom panels, respectively). The mean sensitivity was 72.5% for RF and 68.0% for SVM. The mean specificity was 76.1% for RF and 74.6% for SVM.

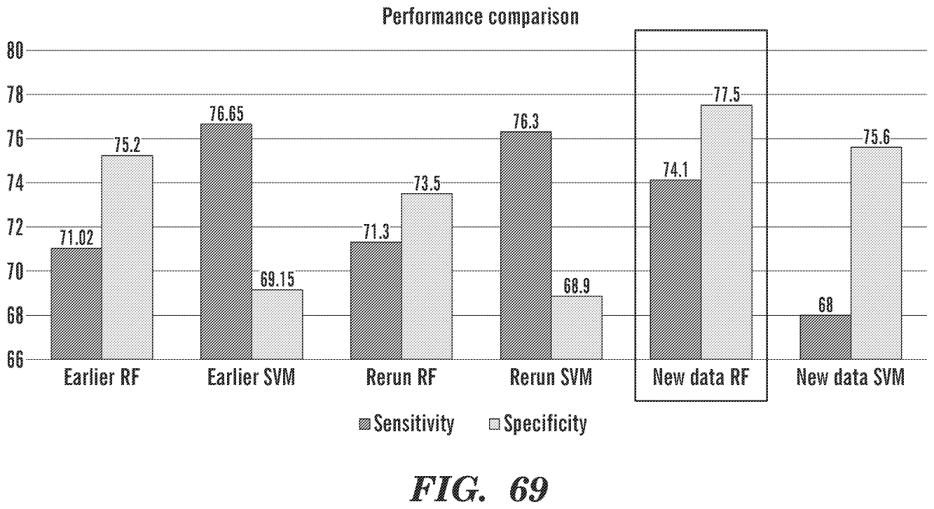

FIG. 69 shows the repeatability analysis and performance comparison for two different operators on dataset 1 (earlier, rerun) as well as performance comparisons for dataset 2. RF on dataset 2 provided the highest and balanced performance on the combined data, with 74.1% sensitivity and 77.5% specificity.

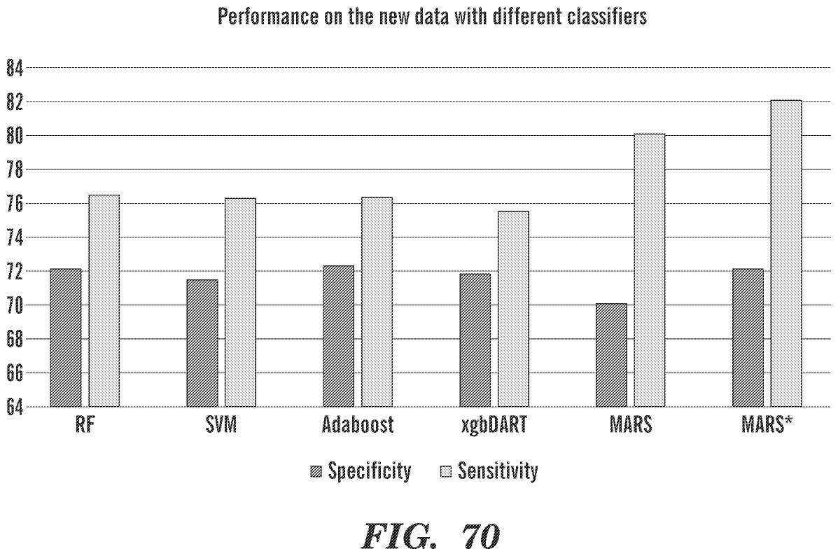

FIG. 70 shows the specificity and sensitivity performance results on data using different classifiers, including RF, SVM, Adaboost, xgbDART, MARS, and MARS*.

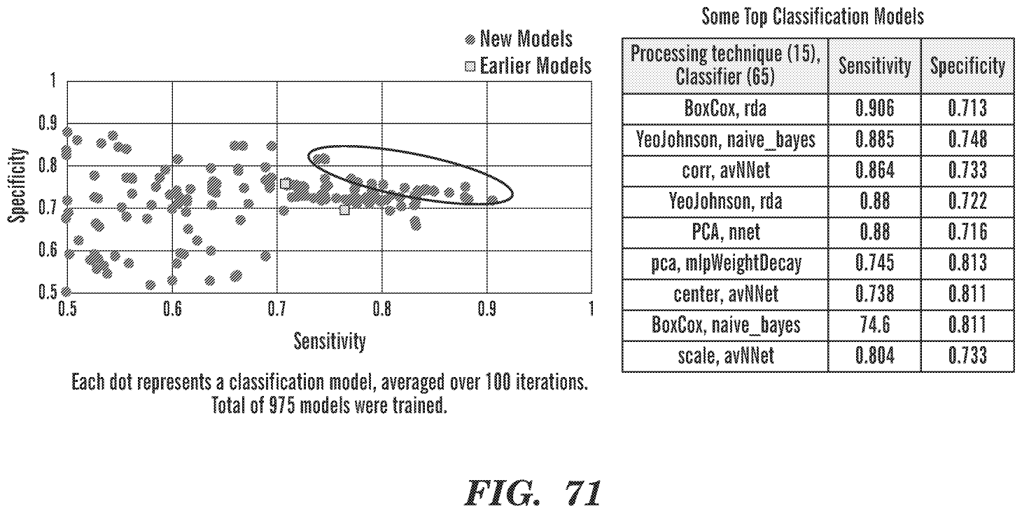

FIG. 71 shows a plot of specificity versus sensitivity and a corresponding table highlighting the top results for specificity and sensitivity using varying preprocessing and classifiers, with BoxCox, rda displaying sensitivity of 90.6% and specificity of 71.3%.

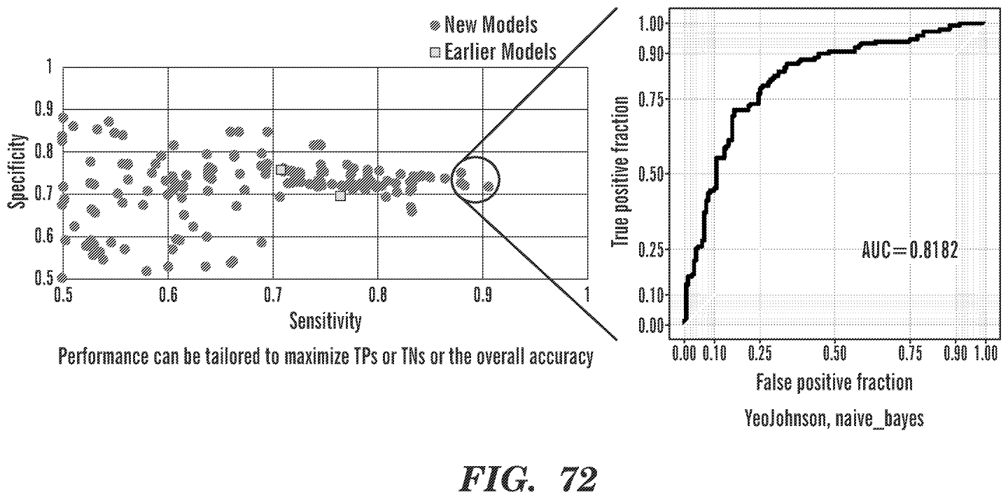

FIG. 72 shows the AUC of the classifier, YeoJohnson, naïve_bayes, as a plot of true positive fraction versus false positive fraction.

### Example 4: A Neural Network Classifying Endometriosis Using Fewer Biomarkers

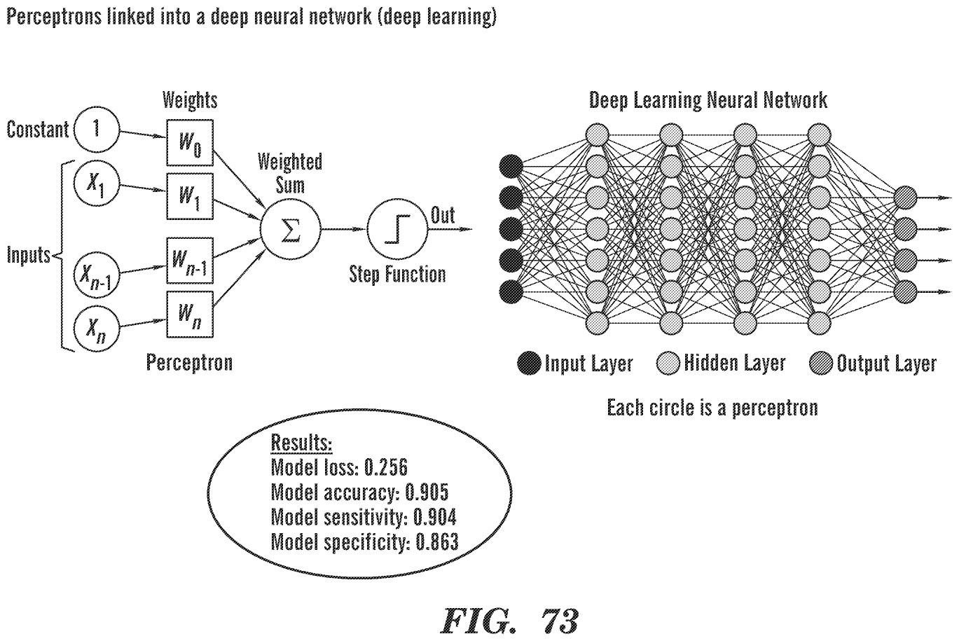

Biomarkers important for classifying endometriosis were identified using the Bristow, 522, and Correlogic datasets. The biomarkers most important for classification were used to prepare a neural network (see FIG. 73). Importance of each biomarker in classifying endometriosis were identified by determining importance of biomarkers in four methods.

The Bristow and 522 datasets have 186 endometriosis patients and 931 non-endometriosis patients. It was observed during clinical testing that a fair number of benign masses were endometriomas or endometriotic cysts. Using the Bristow and 522 datasets, sensitivity and specificity of AdaBoost M1, C5.0, Naïve Bayesian classifier, and Extreme gradient boosted tree classifier methods were determined (FIG. 74). The importance of different biomarkers used in classifying endometriosis in the Bristow and 522 datasets was determined (Table 2). Feature importance was determined using the R Boruta package. The mean was calculated across all four indicated algorithms.

TABLE 2
Feature Importance of Biomarkers of Bristow and 522 Datasets

| | | | | Extreme | |
|---|---|---|---|---|---|
| | | | Naïve | Gradient | |
| | Adaboost | | Bayesian | Boosted | |
| | M1 | C5.0 | Classifier | Trees | Mean |
| CA125 | 100.00 | 100.00 | 100.00 | 11.60 | 77.90 |
| ApoA1 | 61.34 | 100.00 | 0.60 | 21.65 | 45.90 |
| Age | 19.95 | 41.48 | 5.72 | 100.00 | 41.79 |
| TRF | 38.04 | 100.00 | 5.92 | 0.00 | 35.99 |
| B2M | 0.00 | 100.00 | 18.31 | 14.78 | 33.27 |
| FSH | 18.57 | 100.00 | 1.83 | 10.30 | 32.67 |
| HE4 | 3.59 | 100.00 | 0.00 | 5.09 | 27.17 |
| PREA | 12.09 | 0.00 | 13.03 | 0.98 | 6.52 |

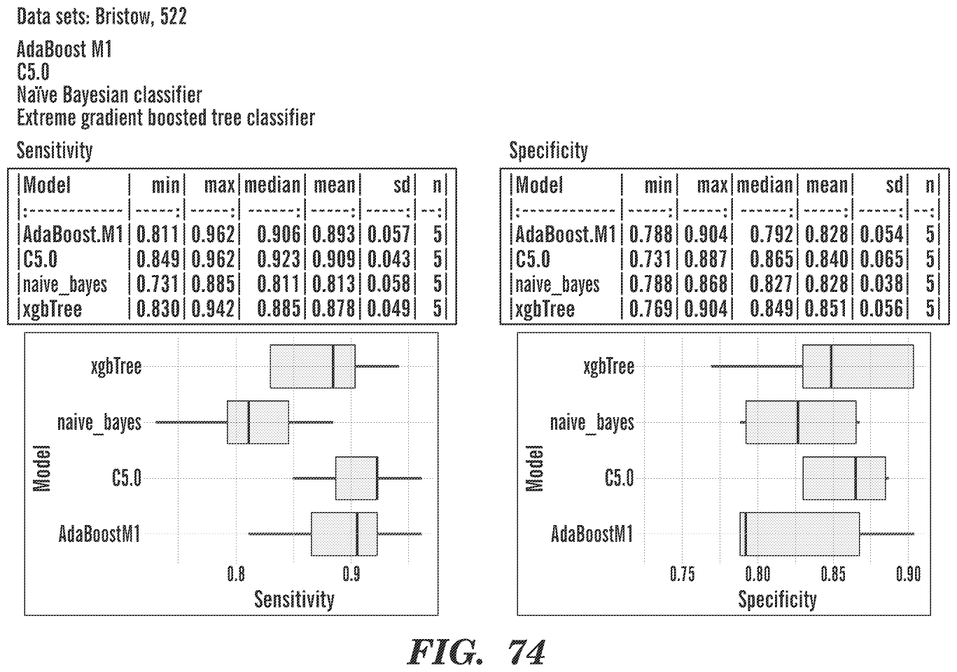

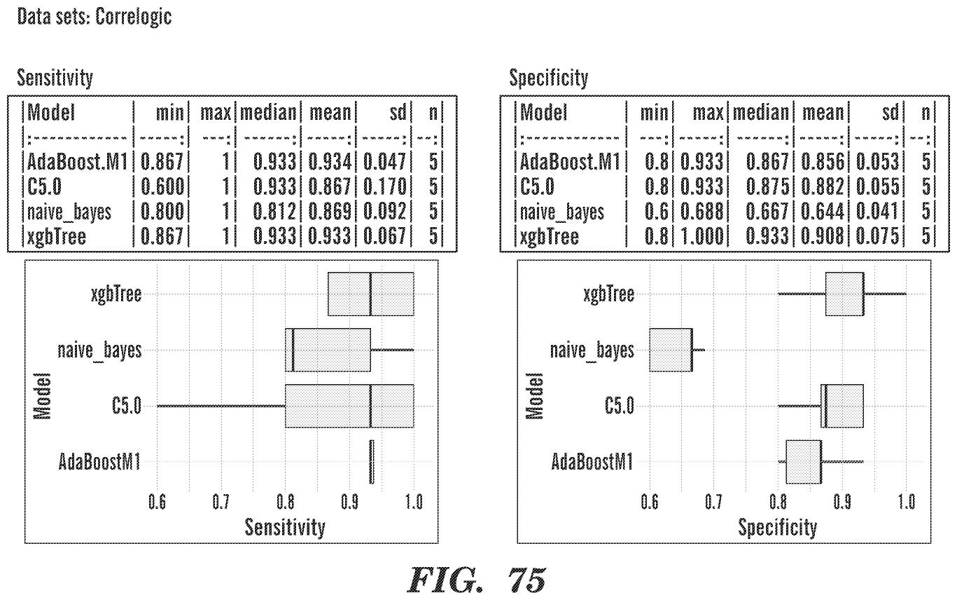

The Correlogic dataset has 53 known endometriosis cases (42 pre-menopausal) and many different biomarkers. Using the Correlogic dataset, sensitivity and specificity of AdaBoost MA, C5.0, Nave Bayesian classifier, and Extreme gradient boosted tree classifier methods were determined (FIG. 75). The importance of different biomarkers used in classifying endometriosis in the Correlogic dataset was determined (Table 3). Feature importance was determined using the R Boruta package. The mean was calculated across all four indicated algorithms.

TABLE 3
Feature Importance of Biomarkers of Correlogic Dataset

| | | | | Extreme | |
|---|---|---|---|---|---|
| | | | Naïve | Gradient | |
| | Adaboost | | Bayesian | Boosted | |
| | M1 | C5.0 | Classifier | Trees | Mean |
| CA125 | 100.00 | 100.00 | 100.00 | 100.00 | 100.00 |
| Age | 47.68 | 100.00 | 41.60 | 11.17 | 50.11 |
| MDC | 34.73 | 100.00 | 34.19 | 14.24 | 45.79 |
| P4 | 28.42 | 100.00 | 43.20 | 10.55 | 45.54 |
| EN-RAGE | 24.66 | 100.00 | 34.31 | 12.67 | 42.91 |
| IgM | 32.30 | 100.00 | 9.65 | 19.41 | 40.34 |
| HCC4 (CCL4) | 0.00 | 88.87 | 49.52 | 5.49 | 35.97 |
| B2M | 23.89 | 88.87 | 0.00 | 15.70 | 32.12 |
| FSH | 17.16 | 100.00 | 2.43 | 9.42 | 30.17 |
| LH | 8.82 | 100.00 | 2.43 | 9.42 | 30.17 |
| CST3 | 19.48 | 0.00 | 4.73 | 19.43 | 10.91 |

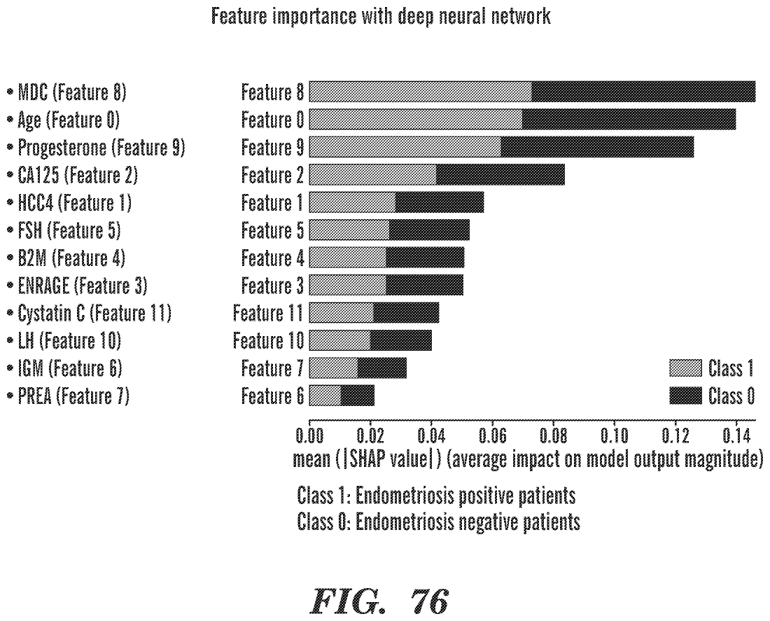

Based upon the above calculations, relative importance of different biomarkers in classifying endometriosis was determined (FIG. 76). Biomarker importance was determined using some of the algorithm methods from FIGS. 71 and 72 and compared to deep neural network calculations corresponding to FIGS. 73 and 76. An objective was to determine if the feature importance seen in the calculations of FIGS. 74 and 75 also held true in the calculations of FIG. 76. Based upon the importance of age, CA125, MDC, Progesterone, and APOA1 to classifying endometriosis, a neural network was prepared using these biomarkers. The resulting neural network had Model loss of 0.2772, Model accuracy of 0.8968, Model sensitivity of 0.9063, and Model specificity of 0.8466.

### Example 5: Study I—Bristow and 522 Datasets (Machine Learning Classifiers for Endometriosis and Endometrioma in Patients with Pelvic Mass)

Sample Characteristics: Archived serum samples from independent prospectively collected sets of specimens from two studies, the OVA2-001-C03 study and PS110001, were used. These data were originally collected to validate two FDA-cleared tests, OVA1 and Overa, both multivariate index assays in detecting risk for ovarian cancer in women who present with an adnexal mass. Consecutive patients who met inclusion criteria of both studies were prospectively enrolled from sites throughout the United States, under Institutional Review Board approval. All enrolling clinicians were from non-gynecological oncology specialty practices, although patients may have had consultation with or undergone surgery by a gynecologic oncologist. Inclusion criteria were the following: women aged 18 years who signed an informed consent, agreed to phlebotomy, and had a documented pelvic mass planned for surgical intervention. A pelvic mass was confirmed by imaging (computed tomography, ultrasonography, or magnetic resonance imaging) prior to enrollment. Exclusion criteria included a diagnosis of malignancy in the previous 5 years (except of nonmelanoma skin cancers). Menopause was defined as the absence of menses for ≥12 consecutive months or age≥50 years. All patients had surgical pathology-based confirmation of diagnosis (confirmed by an independent study pathologist). Table 4 provides the patient demographics summary for Study I.

TABLE 4
Patient demographics summary for Study I (Premenopausal only).

| | |
|---|---|
| N (sample size) | 1117 |

Age (years)

| | |
|---|---|
| Mean | 38.68 |
| Median | 40 |
| Range | 18-60 |

Ethnicity and Race

| | |
|---|---|
| American Indian or Alaska Native | 4 |
| Ashkenazi Jewish | 1 |
| Asian | 28 |
| Black or African American | 172 |
| Hispanic or Latino | 132 |
| Middle Eastern | 2 |
| Native Hawaiian or Other Pacific Islander | 4 |
| White/Caucasian | 770 |
| Other | 4 |

Assay Metrics: A preoperative blood sample of ≤80 mL was processed within 1-6 hours of collection, and serum was frozen at the collection site. Serum samples were shipped to an archive site (PrecisionMed Inc, Solana Beach, Calif.) where they were, after being thawed and aliquoted, consumed entirely during testing. Serum biomarker concentrations were determined on the Roche cobas 6000 clinical analyzer, utilizing the c501 and e601 modules according to the manufacturer's instructions. The c501 module is a photometric detection module for homogenous immunoassays whereas the e601 module is electro-chemiluminescent detection module used for heterogeneous immunoassays. All measurements were performed on coded samples (blinded about patient demographics or pathology outcome) at the Clinical Laboratory Improvement Amendments-/College of American Pathologists-certified laboratory of the Division of Clinical Chemistry, Department of Pathology, Johns Hopkins Medical Institution. Biomarker concentrations for apolipoprotein A-1 (APOA1), B2M, CA125, HE4, FSH, prealbumin (PREA) and TRF were determined from the cobas assays.

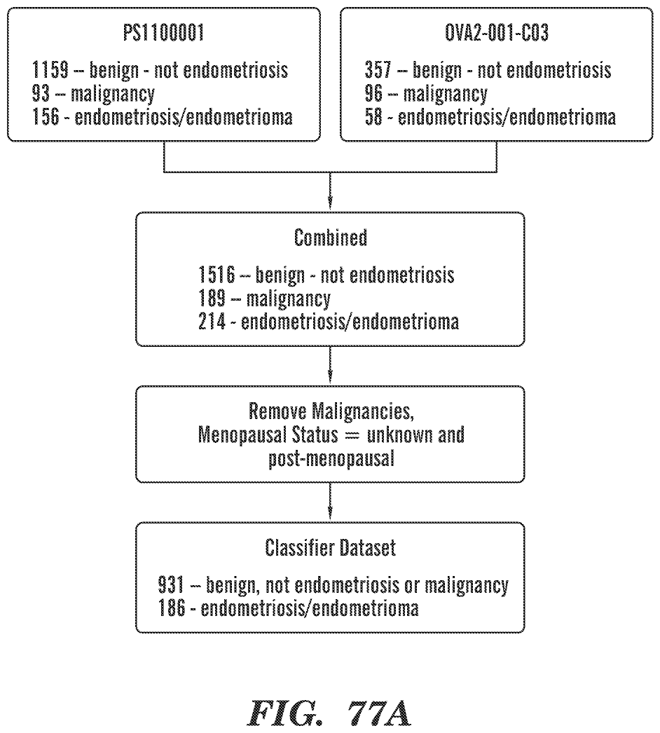

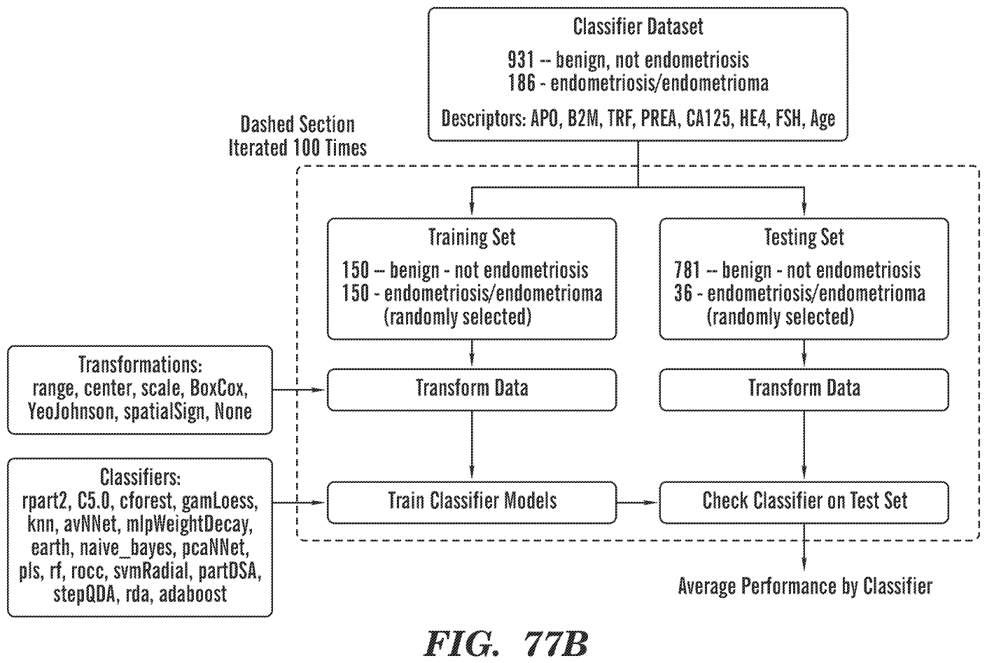

ClinicalMetrics: To determine if established biomarkers in ovarian cancer malignancy risk had utility in identifying patients with endometriosis/endometrioma, machine-language (ML) classifiers were used incorporating these biomarkers and other data (e.g., age) laying the foundation of a proof of concept study (FIGS. 77A and 77B). Data from the two trials were combined to yield the combined Bristow and 522 datasets. Patients that were post-menopausal or of unknown menopausal status were omitted, as were the patients with malignancies. This filtering resulted in 931 non-endometriosis patients and 186 with endometriosis/endometrioma.

Analysis Metrics: Transformation (data normalization) and classifier generation was conducted using the Caret package in the R Statistical Programming Language. To explore a wide algorithm landscape, seven (7) data normalization methods and 18 classification models were used, thus giving a total of 126 different transformation/classifier combinations. For each combination of transformation/classifier, a set of 150 non-endometriosis patients and 150 endometriosis/endometrioma patients was used as training set to create a balanced model between sensitivity and specificity, thus leaving the remaining 36 positive patients and 781 negative patients to form the testing set. For a robust estimate of performance, average sensitivity and specificity was reported over 100 such iterations. The subjects in training and testing patients were different in each iteration and chosen randomly.

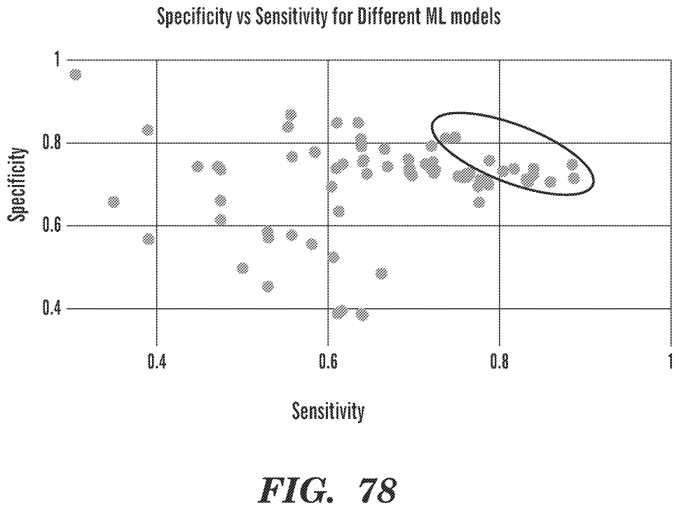

Results for the top models are shown in FIG. 78 and Table 5. Best pre-processing was found using the Yeo-Johnson and Box-Cox power transformations. Naïve-Bayesian and Model Averaged Neural Networks showed better performance than other models. The performance with the best model registered 88.5% sensitivity with 74.8% specificity indicating that these biomarkers have sufficient power to discriminate endometriosis cases in this population.

TABLE 5
Sensitivity and specificity of endometriosis classification
utilizing multiple machine learning algorithms and
data pre-processing transformations

| Pre-processing | Classification | | |
|---|---|---|---|
| Method | Algorithm | Sensitivity | Specificity |
| Yeo-Johnson | Naïve-Bayesian | 88.5% | 74.8% |
| | classifier | | |
| Box-Cox | Model Averaged | 84.0% | 73.9% |
| | Neural Network | | |
| Yeo-Johnson | Regularized | 88.6% | 71.6% |
| | Discriminant | | |
| | Analysis | | |
| Box-Cox | Regularized | 88.8% | 71.5% |
| | Discriminant | | |
| | Analysis | | |
| Box-Cox | Naïve-Bayesian | 74.8% | 81.3% |
| | Classifier | | |
| Data centering | Model Averaged | 73.8% | 81.1% |
| | Neural Network | | |
| Yeo-Johnson | Model Averaged | 81.7% | 73.8% |
| | Neural Network | | |
| Data scaling | Model Averaged | 80.4% | 73.3% |
| | Neural Network | | |

### Example 6: Study II—Correlogic Dataset (Statistical Feature Selection and Deep Neural Network Classification of Endometriosis and Endometrioma in Patients with Pelvic Masses)

Study II was performed to improve upon Study I in two aspects: 1) include additional biologically relevant biomarkers, and 2) explore more classification techniques to improve the performance in classifying endometriosis disease state from non-disease state.

Sample Characteristics: For this study, serum samples were obtained from a prospective collection undertaken by Correlogic Systems, Inc. specifically to develop and validate the performance of an ovarian cancer test. All samples were collected under a uniform protocol from 11 different sites with adherence to IRB approvals. The study inclusion criteria were: women at least 18 years of age, symptomatic of ovarian cancer according to the National Comprehensive Cancer Network (NCCN) Ovarian Cancer Treatment Guidelines for Patients (Agarwal S K, et al. Clinical diagnosis of endometriosis: a call to action. Am J Obstet Gynecol. 2019), with or without a pelvic mass. Participants had to be scheduled for gynecologic surgery based on the concerns that they had ovarian cancer, and post-surgical pathological evaluation of the ovaries and excised tissues was required to establish clinical truth of disease status. Women who did not meet the inclusion criteria, could not provide informed consent, were pregnant, or were previously treated for ovarian cancer were excluded. Written informed consent was obtained for each participant in the study.

Assay Metrics: Study II data (the Correlogic data set) comprised of 53 endometriosis and 124 non-endometriosis patient samples. Table 6 includes the patient demographics summary for Study II. For each sample, 10 ml blood collections were clotted for at least 30 minutes at room temperature, centrifuged at 3,500 g for 10 minutes, and the removed sera was frozen in cryotubes at −80° C. Processing from blood draw to freezing was completed within 2 hours. For biomarker determination, de-identified samples were shipped to Myriad Rules-Based Medicine, Inc. (RBM; Austin, Tex.). A total of 259 different serum biomarkers were measured using a set of proprietary multiplexed immunoassays (Human DiscoveryMAP® v1.0 and Human OncologyMAP® v1.0) at RBM in their CLIA-certified laboratory. Each assay was calibrated using an 8-point standard curve, performed in duplicate.

TABLE 6
Patient demographics summary for Study II.
Patients in initial endometriosis analysis (Premenopausal only)

| | |
|---|---|
| N (sample size) | 177 |

Age (years)

| | |
|---|---|
| Mean | 42.32 |
| Median | 45 |
| Range | 18-60 |

Ethnicity and Race

| | |
|---|---|
| Asian | 8 |
| Black or African American | 18 |
| Hispanic or Latino | 7 |
| Middle Eastern | 3 |
| Native Hawaiian or Other | 2 |
| Pacific Islander | |
| White/Caucasian | 123 |
| Undisclosed | 2 |

Analytical Metrics: Similar to Study I, classification algorithms were built using the biomarker concentrations and other clinical features from the patient data with randomized hold-out cross-validation strategy by dividing the available data into training and testing sets. Values reported for sensitivity and specificity were algorithm predictions on the testing data split.

While Study I focused on developing machine learning algorithms on a specific set of biomarkers, Study II was intended to determine if there were any additional biomarkers from the list of 259 biomarkers that could be used for further development of the algorithm. For this reason, statistical feature selection was performed using the R Boruta package. Variable importance measure was compared using Random Forest (RV).

The 259 biomarkers in Study II, which included all Study I biomarkers, were compared for variable importance. Biomarkers with mean importance of 30.00 or greater (last column) are displayed in Table 7. The different algorithms tested are listed in the middle four columns of Table 7. Like Study I, CA125 and age were the two most notable features (Table 7) suggesting they would improve model performance. Apart from those in Study I, two additional biomarkers displayed high contribution and were selected to be included for further algorithm development: Progesterone (PROG) and Immunoglobulin M (IgM). These two biomarkers were run on the Roche cobas platform in tandem with the other seven biomarkers, which were all FDA-cleared. Macrophage Derived Chemokine (MDC) and EN-RAGE (an S100A12 receptor biomarker) also had high importance, but did not have an FDA-cleared test on the same clinical testing platform, and therefore, were not included in the final analysis. The feature importance from Study II was also confirmed independently using another algorithm (DeepLIFT) thus increasing the confidence in the relevance of these biomarkers (data not shown).

Following the variable importance analysis, the deep neural network (DNN) was trained with six of the seven biomarkers from Study I (APOA1, B2M, CA125, HE4, FSH, and TRF) and adding Progesterone (PROG) and IgM to Study II. Prealbumin was not included in model building as it demonstrated a low importance in both studies. The data was divided into a 60% training, 40% testing set. Like Study I, the features were pre-processed and normalized using the Yeo-Johnson transformation.

TABLE 7
Protein biomarker importance in the data from Study II.

| | | | | Extreme | |
|---|---|---|---|---|---|
| | | | Naïve | Gradient | |
| | Adaboost | | Bayesian | Boosted | |
| Feature | M1 | C5.0 | Classifier | Trees | Mean |
| CA125 | 100 | 100 | 100 | 100 | 100 |
| APOA1 | 78.57 | 96.71 | 71.92 | 17.47 | 66.17 |
| Age | 47.68 | 100 | 41.6 | 11.17 | 50.11 |
| MDC | 34.73 | 100 | 34.19 | 14.24 | 45.79 |
| PROG | 28.42 | 100 | 43.2 | 10.55 | 45.54 |
| EN-RAGE | 24.66 | 100 | 34.31 | 12.67 | 42.91 |
| IGM | 32.3 | 100 | 9.65 | 19.41 | 40.34 |
| HCC4 | 0 | 88.87 | 49.52 | 5.49 | 35.97 |
| B2M | 23.89 | 88.87 | 0 | 15.7 | 32.12 |
| FSH | 17.16 | 100 | 6.13 | 0 | 30.82 |
| LH | 8.82 | 100 | 2.43 | 9.42 | 30.17 |

To compensate for the lower prevalence of confirmed endometriosis cases in the Study II data, the synthetic minority oversampling technique (SMOTE) algorithm was used. The minority class in the training data (expression of endometriosis) was randomly oversampled at 200%. After performing SMOTE algorithm, the DNN was trained using the TensorFlow Keras library for Python and trained 200 times using independent randomized data divisions.

The DNN reported higher mean sensitivity and specificity values than those observed in Study I (Table 8). This increase in performance demonstrated that inclusion of more biologically meaningful biomarkers increases the diagnostic capabilities even in a cohort with endometriosis as a secondary diagnosis.

TABLE 8
Mean sensitivity and specificity for the deep neural
network on the test data division in Study II.

| Pre-Processing | Classification | | |
|---|---|---|---|
| Method | Algorithm | Sensitivity | Specificity |
| Yeo-Johnson | Deep Neural | 90.03 | 82.71 |
| | Network | | |

### Example 7: Study III (Model Performance on Endometriosis Patients Confirmed Via Laparoscopic Visualization)

Study III was performed to verify the performance of the model on a cohort more representative of the intended use population. While the Bristow, 522, and Correlogic datasets used specimens with a known adnexal mass and presenting with symptoms of ovarian cancer (i.e., pelvic pain), Study III included positive endometriosis specimens from a new cohort of women confirmed by surgical laparoscopic visualization. The addition of samples with endometriosis as a primary diagnosis made this a more relevant population set and provided the advantage of introducing more heterogeneity during model building, thereby expanding the prediction power of the classification model.

Sample Characteristics: 150 positive samples, 100 of which were from the new cohort and the remaining 50 specimens were chosen randomly from the Correlogic positive set to increase variability in the model, were investigated. The 100 novel positive samples for Study III were from patients enrolled in a double-blind, randomized, 6-month study. Table 9 includes the additional patient demographics summary for Study III. Specimens were collected from various sites in the United States and Canada from July 2012 through July 2015. Specimens were drawn from premenopausal women between the ages of 18 and 49 years who had received surgical visualization-based diagnosis of endometriosis in the previous 10 years after reporting moderate to severe endometriosis-associated pain (Taylor, Hugh S., et al. “Treatment of Endometriosis-Associated Pain with Elagolix, an Oral GnRH Antagonist.” New England Journal of Medicine 377, no. 1 (May 19, 2017)) To create a balanced negative set, an equal number of negative specimens (150) were randomly selected from Study II.

TABLE 9
Patient demographics summary for Study III - known
endometriosis specimens confirmed by laparoscopic
visualization (Premenopausal only)

| | |
|---|---|
| N (sample size) | 100 |

Age (years)

| | |
|---|---|
| Mean | 30.65 |
| Median | 31 |
| Range | 18-46 |

Ethnicity not disclosed during this study

Assay Metrics: The 100 novel positive samples were 325 μL aliquots of the original patient samples frozen in cryotubes at −80° C. following the conclusion of the double-blind study. For Study III, the patient samples were thawed and transferred into new tubes and shipped on dry ice. The aliquots were received and stored at −20° C. prior to testing. Serum biomarker concentrations were determined using the Roche cobas 6000 clinical analyzer, using the same modules, protocols, and documentation as for Study I. The same biomarkers as tested in Study II were measured: APOA1, B2M, CA125, HE4, Immunoglobulin M (IgM), FSH, Progesterone (PROG) and TRF.

Analytical Metrics: Classification algorithms were built using the biomarker concentrations and other clinical features from the patient data (150 positive and 150 negative samples) with randomized hold-out cross-validation strategy by dividing the data into training (70%) and testing (30%) sets. Values reported for sensitivity and specificity were algorithm predictions on the testing data averaged over 100 iterations.

The addition of the positive samples in Study III into the training and testing of the DNN increased sensitivity to 96.7% and specificity to 92.5% (Table 10). Not wishing to be bound by theory, the higher performance may be due to improvement in both quantity and quality of positive data points: 150 as opposed to 53 in the Correlogic data, and the use of laparoscopically visually confirmed women treated specifically for endometriosis versus women with a pelvic mass that presented symptoms of ovarian cancer who also showed tissue findings of endometrial disease.

TABLE 10
Mean sensitivity and specificity for the deep neural network on
the test data division in Study III specimens in algorithm training
and testing. The metrics are the means of 100 randomized data
divisions and independent algorithm training events.

| Pre-Processing | Classification | | |
|---|---|---|---|
| Method | Algorithm | Sensitivity | Specificity |
| Yeo-Johnson | Deep Neural | 96.77 | 92.52 |
| | Network | | |

Summary: Overall, the performance of the algorithm increased with every development phase and reached the higher end of the widely ranging performance from laparoscopic surgical assessment when tested in the intended use population. Intended use positives in Study III demonstrated the power of a classifier built on a more varied and representative cohort of the disease. In fact, to assess the usefulness of including more relevant and heterogeneous data in Study III, the classification model built in Study II was used on the additional data (i.e., train on Study II data and test on Study III data). As anticipated, the model built on Study II data had a lower performance on Study III samples suggesting that it did not generalize for the intended use group (data not shown). This shows that the algorithm built on a diverse training data is more consistent across different samplings and diagnosis methods.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof. All patents, publications, and accession numbers mentioned in this specification are herein incorporated by reference to the same extent as if each independent patent, publication, and accession number was specifically and individually indicated to be incorporated by reference.

1. A panel for non-invasively characterizing endometriosis in a biological sample of a subject, the panel comprising one of the following sets of polypeptide markers
Apolipoprotein A1 (ApoA1), β2-microglobulin (B2M), Cancer Antigen 125 (CA125), Transferrin (TRF), Transthyretin (TT)/Prealbumin (PREA), Human Epididymis Protein 4 (HE4), Follicle Stimulating Hormone (FSH), and one of the following polypeptide markers: Chemokine 4 (CCL4, MIP-1β), Immunoglobulin M (IgM), Luteinizing Hormone (LH), Macrophage Derived Chemokine (MDC, CCL22), and Progesterone (P4);
β2-microglobulin (B2M), Cancer Antigen 125 (CA125), Follicle Stimulating Hormone (FSH), Chemokine 4 (CCL4, MIP-1β), Immunoglobulin M (IgM), Luteinizing Hormone (LH), Macrophage Derived Chemokine (MDC, CCL22), and Progesterone (P4) or polynucleotides encoding such polypeptides;
Cancer Antigen 125 (CA125), Apolipoprotein A1 (ApoA1), Transferrin (TRF), β2-microglobulin (B2M), Follicle Stimulating Hormone (FSH), Human Epididymis Protein 4 (HE4), and Prealbumin (PREA);
Cancer Antigen 125 (CA125), Macrophage Derived Chemokine (MDC, CCL22), Progesterone (P4), EN-RAGE (S100A12), Immunoglobulin M (IgM), Chemokine 4 (CCL4, MIP-1β), (2-microglobulin (B2M), Follicle Stimulating Hormone (FSH), Luteinizing Hormone (LH), and Cystatin C (CST3);
Cancer Antigen 125 (CA125), Macrophage Derived Chemokine (MDC, CCL22), Progesterone (P4), and Apolipoprotein A1 (ApoA1);
Cancer Antigen 125 (CA125), Apolipoprotein A1 (ApoA1), Macrophage Derived Chemokine (MDC, CCL22), Progesterone (P4), EN-RAGE (S100A12), Immunoglobulin M (IgM), Chemokine 4 (CCL4, MIP-1β, HCC4), 12-microglobulin (B2M), Follicle Stimulating Hormone (FSH), Luteinizing Hormone (LH); and
Apolipoprotein A1 (ApoA1), β2-microglobulin (B2M), Cancer Antigen 125 (CA125), Transferrin (TRF), Transthyretin (TT)/Prealbumin (PREA), Human Epididymis Protein 4 (HE4), Follicle Stimulating Hormone (FSH) and one or more of the following polypeptide markers: Alpha 1 Microglobulin (AIM), Alpha Fetoprotein (AFP), Angiopoietin 2 (Ang-2), Apolipoprotein B (ApoB), Apolipoprotein E (ApoE), Cystatin C (CST3), CD 40 Antigen (CD40), Chromogranin A (CgA), Chemokine 4 (CCL4, MIP-1), Clusterin (CLU, ApoJ), Eotaxin 1 (CCL11), Endostatin, EN-RAGE (S100A12), Fatty Acid Binding Protein (adipocyte)(FABP4), Fatty Acid Binding Protein (heart) (H-FABP, FABP3), Fas Ligand Receptor (Fas, FasR), Ferritin (FTL), Galectin 3 (Gal-3), Growth Hormone (GH, somatotropin, human growth hormone, HGH), Glutathione S Transferase alpha (GSTα), Human Chorionic Gonadotropin (hCG), Hepatocyte Growth Factor (HGF), Haptoglobin (Hp), Immunoglobulin E (IgE), Insulin like Growth Factor Binding Protein 4 (IGFBP4), Insulin like Growth Factor I (IGF-I), Immunoglobulin M (IgM), Interleukin 8 (IL-8, CXCL8), Interferon gamma Induced Protein 10 (IP-10, CXCL10), Interferon inducible T cell alpha chemoattractant (I-TAC, CXCL11), Kallikrein 5 (KLK5), Leptin (LEP), Luteinizing Hormone (LH), Monocyte Chemotactic Protein 1 (MCP-1, CCL2), Monocyte Chemotactic Protein 4 (MCP-4, CCL13), Macrophage Derived Chemokine (MDC, CCL22), Monokine Induced by Gamma Interferon (Mig, CXCL9), Macrophage Inflammatory Protein 1 alpha (MIP-1α, CCL3), Matrix Metalloproteinase 3 (MMP-3), Myoglobin (Mb), N terminal prohormone of brain natriuretic peptide (NT-proBNP), Osteoprotegerin (OPG, TNFRSF11B), Pulmonary and Activation Regulated Chemokine (PARC), Prostasin (PRSS8), Phosphoserine Aminotransferase (PSAT), Stem Cell Factor (SCF), Thymus Expressed Chemokine (TECK), Trefoil Factor 3 (TFF3), Tumor Necrosis Factor alpha (TNFα, TNF), Tumor Necrosis Factor Receptor 1 (TNFR1), Tumor Necrosis Factor Receptor 2 (TNFR2), Tissue type Plasminogen activator (tPA/PLAT), Urokinase type Plasminogen Activator (uPA), Urokinase type Plasminogen Activator Receptor (uPAR, CD87), Vascular Endothelial Growth Factor (VEGF), von Willebrand Factor (VWF), YKL-40 (CHI3L1), or polynucleotides encoding such polypeptides.

2-7. (canceled)

8. The panel of claim 1, wherein the markers are bound to a capture molecule.

9. The panel of claim 8, wherein the capture molecule is bound to a substrate.

10. A panel of capture molecules, wherein each capture molecule binds a polypeptide biomarker of claim 1.

11. The panel of claim 8, wherein the capture molecule is an antibody.

12. The panel of claim 8, wherein the capture molecule is a polynucleotide.

13. A method of treating a selected subject, the method comprising administering to the subject a gonadotropin-releasing hormone (GnRH) antagonist or a GnRH agonist, wherein the subject is selected by characterizing a biological sample of the subject as having an alteration in the level of a biomarker relative to a reference, wherein the biomarker is selected from the group consisting of Apolipoprotein A1 (ApoA1), β2-microglobulin (B2M), Cancer Antigen 125 (CA125), Transferrin (TRF), Transthyretin (TT)/Prealbumin (PREA), Human Epididymis Protein 4 (HE4), Follicle Stimulating Hormone (FSH), and one or more of the following polypeptide markers: Chemokine 4 (CCL4, MIP-1β), Immunoglobulin M (IgM), Luteinizing Hormone (LH), Macrophage Derived Chemokine (MDC, CCL22), and Progesterone (P4);
β2-microglobulin (B2M), Cancer Antigen 125 (CA125), Follicle Stimulating Hormone (FSH), Chemokine 4 (CCL4, MIP-1β), Immunoglobulin M (IgM), Luteinizing Hormone (LH), Macrophage Derived Chemokine (MDC, CCL22), and Progesterone (P4) or polynucleotides encoding such polypeptides;
Cancer Antigen 125 (CA125), Apolipoprotein A1 (ApoA1), Transferrin (TRF), β2-microglobulin (B2M), Follicle Stimulating Hormone (FSH), Human Epididymis Protein 4 (HE4), and Prealbumin (PREA);
Cancer Antigen 125 (CA125), Macrophage Derived Chemokine (MDC, CCL22), Progesterone (P4), EN-RAGE (S100A12), Immunoglobulin M (IgM), Chemokine 4 (CCL4, MIP-1β), β2-microglobulin (B2M), Follicle Stimulating Hormone (FSH), Luteinizing Hormone (LH), and Cystatin C (CST3):
Cancer Antigen 125 (CA125), Macrophage Derived Chemokine (MDC, CCL22), Progesterone (P4), and Apolipoprotein A1 (ApoA1);
Cancer Antigen 125 (CA125), Apolipoprotein A1 (ApoA1), Macrophage Derived Chemokine (MDC, CCL22), Progesterone (P4), EN-RAGE (S100A12), Immunoglobulin M (IgM), Chemokine 4 (CCL4, MIP-1β, HCC4), β2-microglobulin (B2M), Follicle Stimulating Hormone (FSH), Luteinizing Hormone (LH);
Apolipoprotein A1 (ApoA1), β2-microglobulin (B2M), Cancer Antigen 125 (CA125), Transferrin (TRF), Transthyretin (TT)/Prealbumin (PREA), Human Epididymis Protein 4 (HE4), Follicle Stimulating Hormone (FSH) and one or more of the following polypeptide markers: Alpha 1 Microglobulin (A1M), Alpha Fetoprotein (AFP), Angiopoietin 2 (Ang-2), Apolipoprotein B (ApoB), Apolipoprotein E (ApoE), Cystatin C (CST3), CD 40 Antigen (CD40), Chromogranin A (CgA), Chemokine 4 (CCL4, MIP-1β), Clusterin (CLU, ApoJ), Eotaxin 1 (CCL11), Endostatin, EN-RAGE (S100A12), Fatty Acid Binding Protein (adipocyte)(FABP4), Fatty Acid Binding Protein (heart) (H-FABP, FABP3), Fas Ligand Receptor (Fas, FasR), Ferritin (FTL), Galectin 3 (Gal-3), Growth Hormone (GH, somatotropin, human growth hormone, HGH), Glutathione S Transferase alpha (GSTα), Human Chorionic Gonadotropin (hCG), Hepatocyte Growth Factor (HGF), Haptoglobin (Hp), Immunoglobulin E (IgE), Insulin like Growth Factor Binding Protein 4 (IGFBP4), Insulin like Growth Factor I (IGF-I), Immunoglobulin M (IgM), Interleukin 8 (IL-8, CXCL8), Interferon gamma Induced Protein 10 (IP-10, CXCL10), Interferon inducible T cell alpha chemoattractant (I-TAC, CXCL11), Kallikrein 5 (KLK5), Leptin (LEP), Luteinizing Hormone (LH), Monocyte Chemotactic Protein 1 (MCP-1, CCL2), Monocyte Chemotactic Protein 4 (MCP-4, CCL13), Macrophage Derived Chemokine (MDC, CCL22), Monokine Induced by Gamma Interferon (Mig, CXCL9), Macrophage Inflammatory Protein 1 alpha (MIP-1α, CCL3), Matrix Metalloproteinase 3 (MMP-3), Myoglobin (Mb), N terminal prohormone of brain natriuretic peptide (NT-proBNP), Osteoprotegerin (OPG, TNFRSF11B), Pulmonary and Activation Regulated Chemokine (PARC), Prostasin (PRSS8), Phosphoserine Aminotransferase (PSAT), Stem Cell Factor (SCF), Thymus Expressed Chemokine (TECK), Trefoil Factor 3 (TFF3), Tumor Necrosis Factor alpha (TNFα, TNF), Tumor Necrosis Factor Receptor 1 (TNFR1), Tumor Necrosis Factor Receptor 2 (TNFR2), Tissue type Plasminogen activator (tPA/PLAT), Urokinase type Plasminogen Activator (uPA), Urokinase type Plasminogen Activator Receptor (uPAR, CD87), Vascular Endothelial Growth Factor (VEGF), von Willebrand Factor (VWF), YKL-40 (CHI3L1), or polynucleotides encoding such polypeptides.

14-19. (canceled)

20. The method of claim 13, further comprising characterizing the age, pre-menopausal or post-menopausal status, of the subject;

21. (canceled)

22. The method of claim 13, wherein the GnRH antagonist is elagolix, abarelix, cetrorelix, degarelix, ganirelix, or relugolix.

23. The method of claim 13, wherein the GnRH antagonist is elagolix.

24. The method of claim 13, wherein the GnRH agonist is goserelin, leuprolide, nafarelin, buserelin, gonadorelin, histrelin, or triptorelin.

25. The method of claim 13, wherein an increase or decrease in the level of one or more of said markers distinguishes endometriosis from non-endometriosis.

26. (canceled)

27. The method of claim 13, wherein the reference is a corresponding biological sample derived from a healthy subject or derived from the same subject at an earlier point in time.

28. (canceled)

29. The method of claim 13, wherein the characterizing step is an immunoassay or affinity capture.

30. The method of claim 29, wherein the immunoassay comprises affinity capture assay, immunometric assay, heterogeneous chemiluminscence immunometric assay, homogeneous chemiluminscence immunometric assay, ELISA, western blotting, radioimmunoassay, magnetic immunoassay, real-time immunoquantitative PCR (iqPCR) and SERS label free assay.

31. A method for determining the marker profile of a biological sample, the method comprising quantifying the levels of a marker of Table 1 in the sample.

32. The method of claim 13, wherein the biological sample is a biological fluid selected from the group consisting of blood, blood serum, and plasma.

33. The method of claim 13, wherein the method is carried out in a plate, chip, beads, microfluidic platform, membrane, planar microarray, or suspension array.

34. The method of claim 13, wherein the method detects a CA125 glycoform.

35. A kit for detecting endometriosis in a biological sample, the kit comprising a set of capture molecules each of which specifically binds a marker of Table 1 or of claim 1.
